(19) United States
(12) Patent Application Publication
(10) Pub. No.: US 2012/0058918 A1
(43) Pub. Date: Mar. 8, 2012

(54) CELL LINES EXPRESSING CFTR AND METHODS OF USING THEM

(75) Inventors: Kambiz Shekdar, New York, NY (US); Jessica Langer, Highland Park, NJ (US); Srinivasan P. Venkatachalan, Milltown, NJ (US); Dennis J. Sawchuk, Fanwood, NJ (US)

(73) Assignee: CHROMOCELL CORPORATION, North Brunswick, NJ (US)

(21) Appl. No.: 13/147,327

(22) PCT Filed: Feb. 1, 2010
(86) PCT No.: PCT/US10/22778

§ 371 Date: Aug. 1, 2011

Related U.S. Application Data
(60) Provisional application No. 61/149,312, filed on Feb. 2, 2009.

(51) Int. Cl. C40B 30/06; C12Q 1/02; C12N 5/10

(52) U.S. Cl. 506/10; 435/325; 435/361; 435/375; 435/29

(57) ABSTRACT

Disclosed herein are cells and cell lines that stably express CFTR and methods for using those cells and cell lines. The invention also includes techniques for creating these cells and cell lines. The cells and cell lines of this invention are physiologically relevant. They are highly sensitive and provide consistent and reliable results in cell-based assays.

This application claims the benefit of U.S. Provisional Application 61/149,312, filed Feb. 2, 2009, the contents of which are incorporated herein by reference in their entirety.

### SEQUENCE LISTING

The instant application contains a Sequence Listing which has been submitted via EFS-Web and is hereby incorporated by reference in its entirety. Said ASCII copy, created on Mar. 16, 2010, is named 002298WO.txt and is 40,540 bytes in size.

### FIELD OF THE INVENTION

The invention relates to cystic fibrosis transmembrane conductance regulator (CFTR) and cells and cell lines stably expressing CFTR. The invention further provides methods of making such cells and cell lines. The CFTR-expressing cells and cell lines provided herein are useful in identifying modulators of CFTR.

### BACKGROUND

Cystic fibrosis is the most common genetic disease in the United States, and is caused by mutations in the gene encoding the cystic fibrosis transmembrane conductance regulator (CFTR) protein. CFTR is a transmembrane ion channel protein that transports chloride ions and other anions. The chloride channels are present in the apical plasma membranes of epithelial cells in the lung, sweat glands, pancreas, and other tissues. CFTR regulates ion flux and helps control the movement of water in tissues and maintain the fluidity of mucus and other secretions. Chloride transport is induced by an increase in cyclic adenosine monophosphate (cAMP), which activates protein kinase A to phosphorylate the channel on the regulatory “R” domain.

CFTR is a member of the ABC transporter family. It contains two ATP-binding cassettes. ATP binding, hydrolysis and cAMP-dependent phosphorylation are required for channel opening. CFTR is encoded by a single large gene consisting of 24 exons. CFTR ion channel function is associated with a wide range of disorders, including cystic fibrosis, congenital absence of the vas deferens, secretory diarrhea, and emphysema. To date, more than 1000 distinct mutations have been identified in CFTR. The most common CFTR mutation is deletion of phenylalanine at residue 508 (ΔF508) in its amino acid sequence. This mutation is present in approximately 70% of cystic fibrosis patients.

The discovery of new and improved therapeutics that specifically target CFTR has been hampered by the lack of robust, physiologically relevant cell-based systems that are amenable to high-throughput formats for identifying and testing CFTR modulators, particularly high-throughput formats that allow various members of the CFTR family of mutants to be compared. Cell-based systems are preferred for drug discovery and validation because they provide a functional assay for a compound as opposed to cell-free systems, which only provide a binding assay. Moreover, cell-based systems have the advantage of simultaneously testing cytotoxicity. Ideally, cell-based systems should so stably express the target protein. It is also desirable for a cell-based system to be reproducible. The present invention addresses these problems.

### SUMMARY OF THE INVENTION

We have discovered new and useful cells and cell lines and collections of cell lines that express various forms of CFTR. These cells, cell lines, and collections thereof are useful in cell-based assays, in particular high-throughput assays to study the functions of CFTR and to screen for CFTR modulators.

Accordingly, the invention provides a cell or cell line engineered to stably express CFTR, e.g., a functional CFTR or a mutant (e.g., dysfunctional) CFTR. In some embodiments, the CFTR is expressed in a cell from an introduced nucleic acid encoding it. In some embodiments, the CFTR is expressed in a cell from an endogenous nucleic acid activated by engineered gene activation.

The cells or cell lines of the invention may be eukaryotic cells (e.g., mammalian cells), and optionally do not express CFTR endogenously (or in the case of gene activation, do not express CFTR endogenously prior to gene activation). The cells may be primary or immortalized cells, may be cells of, for example, primate (e.g., human or monkey), rodent (e.g., mouse, rat, or hamster), or insect (e.g., fruit fly) origin. In some embodiments, the cells are capable of forming polarized monolayers. The CFTR expressed in the cells or cell lines of the invention may be mammalian, such as rat, mouse, rabbit, goat, dog, cow, pig, or primate (e.g., human).

In some embodiments, the cells and cell lines of the invention have a Z′ factor of at least 0.4, 0.45, 0.5, 0.55, 0.6, 0.65, 0.7, 0.75, 0.8 or 0.85 in an assay, for example, a high throughput cell-based assay. In some embodiments, the cells or cell lines of the invention are maintained in the absence of selective pressure, e.g., antibiotics. In some embodiments, the CFTR expressed by the cells or cell lines does not comprise any polypeptide tag. In some embodiments, the cells or cell lines do not express any other introduced protein, including auto-fluorescent proteins (e.g., yellow fluorescent protein (YFP) or variants thereof).

In some embodiments, the cells or cell lines of the invention stably express CFTR at a consistent level in the absence of selective pressure for at least 15 days, 30 days, 45 days, 60 days, 75 days, 100 days, 120 days, or 150 days.

In another aspect of the invention, the cells or cell lines express a human CFTR. The CFTR may be a polypeptide having the amino acid sequence set forth in SEQ ID NO: 2; a polypeptide at least 95% sequence identity to SEQ ID NO: 2; a polypeptide encoded by a nucleic acid that hybridizes to SEQ ID NO: 1 under stringent conditions; or a polypeptide that is an allelic variant of SEQ ID NO: 2. The CFTR may also be encoded by a nucleic acid having the sequence set forth in SEQ ID NO: 1; a nucleic acid that hybridizes to SEQ ID NO: 1 under stringent conditions; a nucleic acid that encodes the polypeptide of SEQ ID NO: 2; a nucleic acid with at least 95% sequence identity to SEQ ID NO: 1; or a nucleic acid that is an allelic variant of SEQ ID NO: 1. The CFTR may be a polypeptide having the amino acid sequence set forth in SEQ ID NO: 7 or a polypeptide encoded by a nucleic acid sequence set forth in SEQ ID NO: 4.

In another aspect, the invention provides a collection of the cells or cells lines that express different forms (i.e., mutant forms) of CFTR. In some embodiments, the cells or cell lines in the collection comprise at least 2, at least 5, at least 10, at least 15, or at least 20 different cells or cell lines, each expressing at least a different form (i.e., mutant thrill) of CFTR. In some embodiments, the cells or cell lines in the collection are matched to share physiological properties (e.g., cell type, metabolism, cell passage (age), growth rate, adherence to a tissue culture surface, Z′ factor, expression level of CFTR) to allow parallel processing and accurate assay readouts. These can be achieved by generating and growing the cells and cell lines under identical conditions, achievable by, e.g., automation. In some embodiments, the Z′ factor is determined in the absence of a protein trafficking corrector. A protein trafficking corrector is a substance that aids maturation of improperly folded CFTR mutant by directly or indirectly interacting with the mutant CFTR at its transmembrane level and facilitates the mutant CFTR to reach the cell membrane.

In another aspect, the invention provides a method for producing the cells or cell lines of the invention, comprising the steps of: (a) introducing a vector comprising a nucleic acid encoding CFTR (e.g., human CFTR) into a host cell; or introducing one or more nucleic acid sequences that activate expression of endogenous CFTR (e.g., human CFTR); (b) introducing a molecular beacon or fluorogenic probe that detects the expression of CFTR into the host cell produced in step (a); and (c) isolating a cell that expresses CFTR. In some embodiments, the method comprises the additional step of generating a cell line from the cell isolated in step (c). The host cells may be eukaryotic cells such as mammalian cells, and may optionally do not express CFTR endogenously.

In some embodiments, the method of producing cells and cell lines of the invention utilizes a fluorescence activated cell sorter to isolate a cell that expresses CFTR. In some embodiments, the cell or cell lines of the collection are produced in parallel.

In another aspect, the invention provides a method for identifying a modulator of a CFTR function, comprising the steps of exposing a cell or cell line of the invention or a collection of the cell lines to a test compound; and detecting in a cell a change in a CFTR function, wherein a change indicates that the test compound is a CFTR modulator. In some embodiments, the detecting step can be a membrane potential assay, a yellow fluorescent protein (YFP) quench assay, an electrophysiology assay, a binding assay, or an Ussing chamber assay. In some embodiments, the assay in the detecting step is performed in the absence of a protein trafficking corrector. Test compounds used in the method may include a small molecule, a chemical moiety, a polypeptide, or an antibody. In other embodiments, the test compound may be a library of compounds. The library may be a small molecule library, a combinatorial library, a peptide library, or an antibody library.

In a further aspect, the invention provides a cell engineered to stably express CFTR at a consistent level over time. The cell may be made by a method comprising the steps of: a) providing a plurality of cells that express mRNA(s) encoding the CFTR; b) dispersing the cells individually into individual culture vessels, thereby providing a plurality of separate cell cultures; c) culturing the cells under a set of desired culture conditions using automated cell culture methods characterized in that the conditions are substantially identical for each of the separate cell cultures, during which culturing the number of cells per separate cell culture is normalized, and wherein the separate cultures are passaged on the same schedule; d) assaying the separate cell cultures to measure expression of the CFTR at least twice; and e) identifying a separate cell culture that expresses the CFTR at a consistent level in both assays, thereby obtaining said cell.

In another aspect, the invention provides a method for isolating a cell that endogenously expresses CFTR, comprising the steps of: a) providing a population of cells; b) introducing into the cells a molecular beacon that detects expression of CFTR; and c) isolating cells that express CFTR. In some embodiments, the population of cells comprises cells that do not endogenously express CFTR. In some embodiments, the isolated cells that express CFTR prior to said isolating are not known to express CFTR. In some embodiments, the method further comprises, prior to said isolating step c), the step of increasing genetic variability.

In another aspect, the invention provides a use of a composition comprising a compound of the formula:

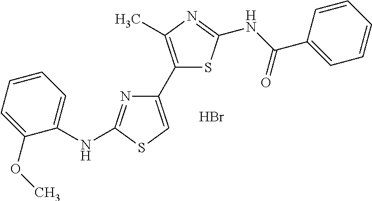

### N-{2-[(2-methoxyphenyl)amino]-4′-methyl-4,5′-bi-1,3-thiazol-2′-yl}benzamide hydrobromide

to increase the expression level of a CFTR on the cell plasma membrane.

### BRIEF DESCRIPTION OF THE FIGURES

FIGS. 1A and 1B show that stable CFTR-expressing cell lines produced exhibit significantly enhanced and robust CFTR surface expression. Ion-flux in response to activated CFTR expression was measured by a high-throughput compatible fluorescence membrane potential assay. FIG. 1A compares stable CFTR-expressing cell line 1 to transiently CFTR-transfected cells and control cells lacking CFTR. FIG. 1B compares stable CFTR-expressing cell line 1 (from FIG. 1A) to other stable CFTR-expressing clones produced (M11, J5, E15, and O1).

FIG. 2 displays dose response curves from a high-throughput compatible fluorescence membrane potential assay of CFTR. The assay measured the response of produced stable CFTR-expressing cell lines to forskolin, an agonist of CFTR. The EC₅₀ value for forskolin in the tested cell lines as 256 nM. A Z′ value of at least 0.82 was obtained for the high-throughput compatible fluorescence membrane potential assay.

FIGS. 3A-3F show that stable CFTR-ΔF508 expressing CHO cell clones can be identified from non-responding clones from a population of CHO cells. Stable CFTR-ΔF508 expressing clones were able to rescue cell surface expression of CFTR-ΔF508 from entrapment in intracellular compartments, in the presence or absence of a protein trafficking corrector—Chembridge compound #5932794a (San Diego, Calif.). This compound is N-{2-[(2-methoxyphenyl)amino]-4′-methyl-4,5′-bi-1,3-thiazol-2′-yl}benzamide hydrobromide, and has the formula of

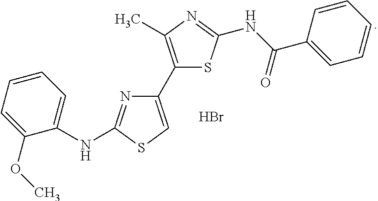

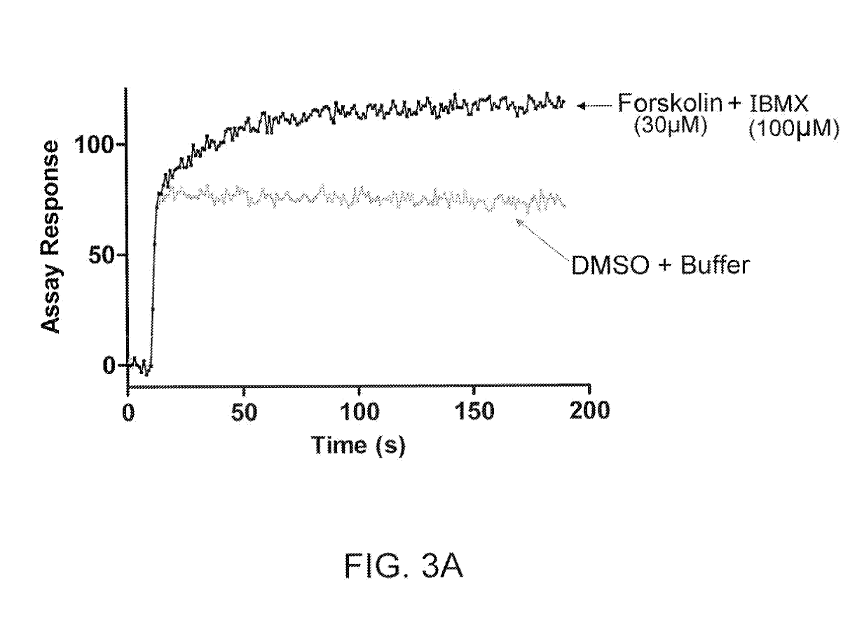

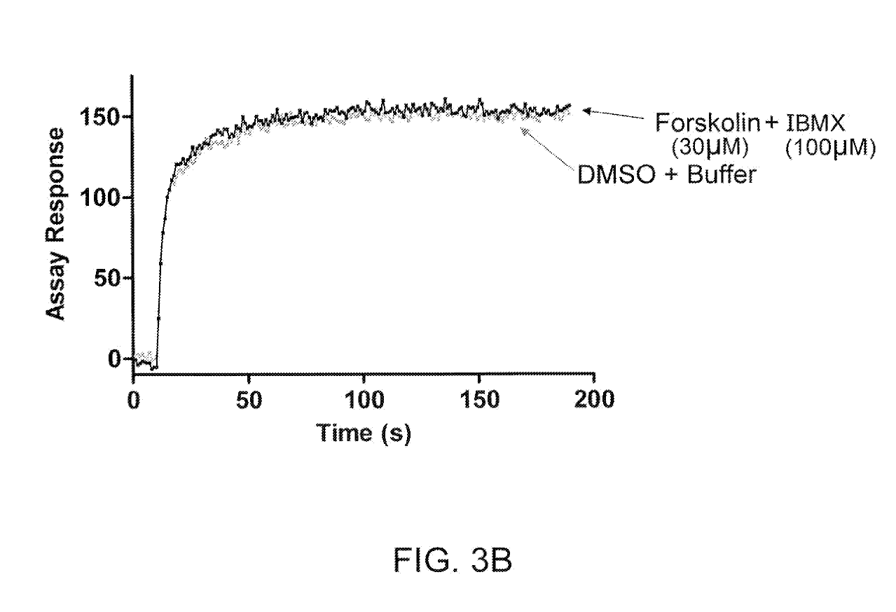

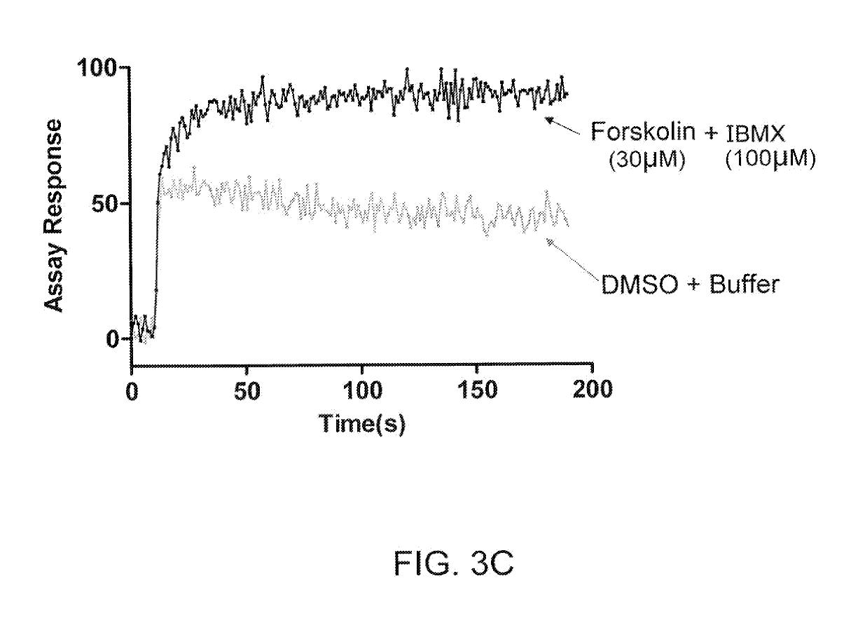

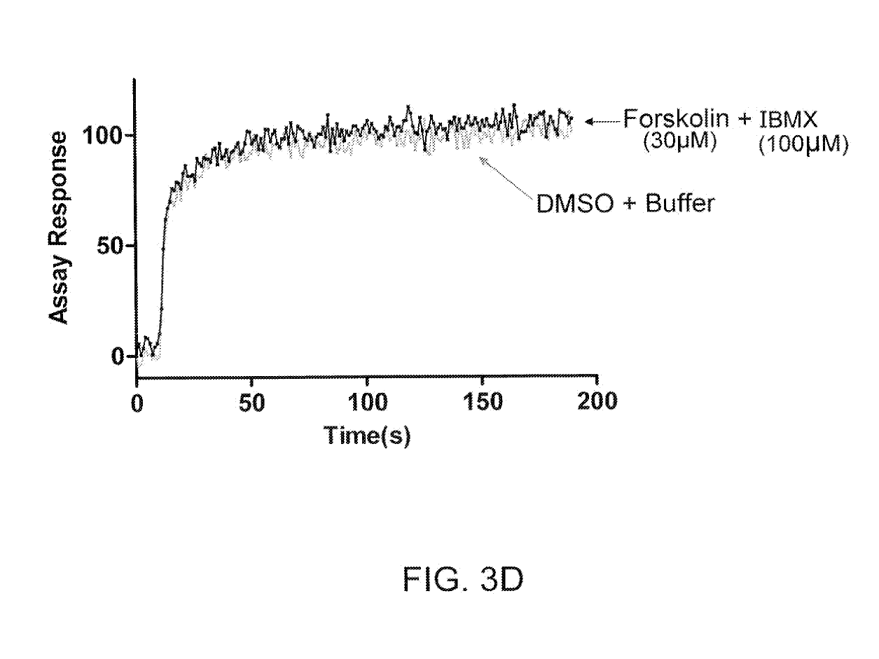

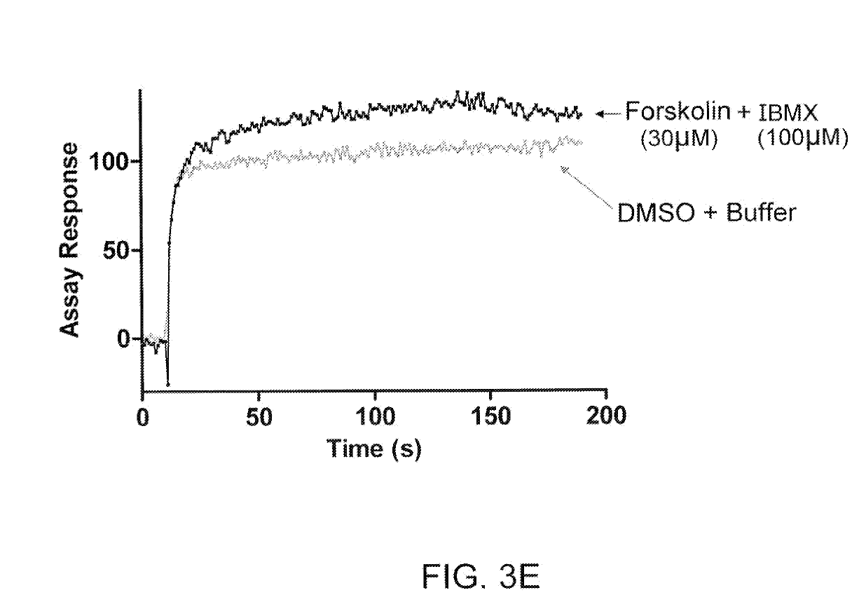

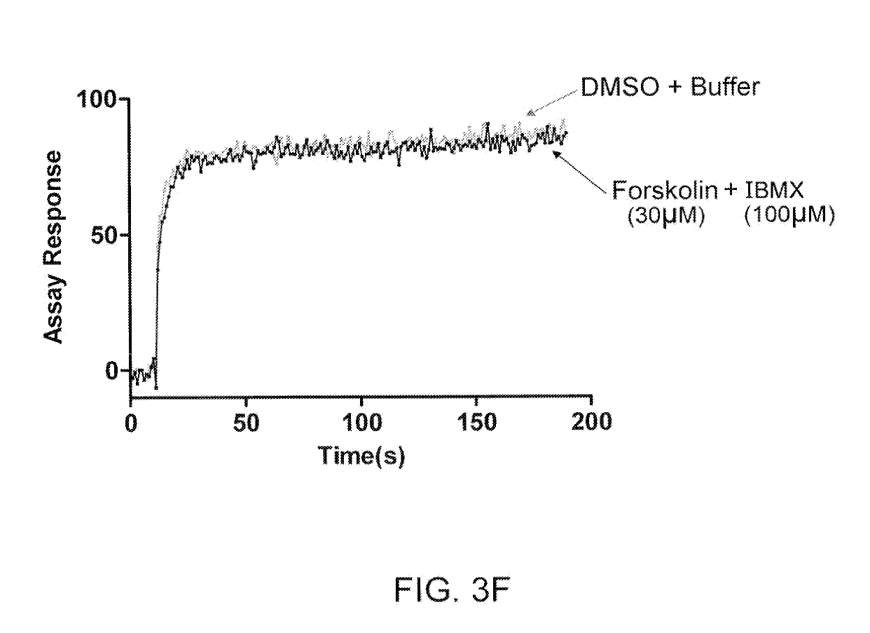

Non-responding clones were not able to rescue cell surface expression of CFTR-ΔF508 from entrapment in intracellular compartments, either in the presence or absence of the protein trafficking corrector. Ion-flux in response to activated CFTR-ΔF508 expression was measured by a high-throughput compatible fluorescence membrane potential assay. FIG. 3A shows pharmacological response of a stable CFTR-ΔF508 expressing clone in the presence of a blue membrane potential dye and the protein trafficking corrector (15-25 μM) when challenged either by an agonist cocktail of forskolin (30 μM)+IBMX (100 μM) (black trace) or DMSO+Buffer (grey trace). FIG. 3B shows pharmacological response of a non-responding clone in the presence of a blue membrane potential dye and the protein trafficking corrector (15-25 μM, same as in 3A) when challenged either by an agonist cocktail of forskolin (30 μM)+IBMX (100 μM) (black trace) or DMSO+Buffer (grey trace). FIG. 3C shows pharmacological response of a stable CFTR-ΔF508 expressing clone in the presence of an AnaSpec membrane potential dye and the protein trafficking corrector (15-25 μM, same as in 3A, 3B) when challenged either by an agonist cocktail of forskolin (30 μM)+IBMX (100 μM) (black trace) or DMSO+Buffer (grey trace). FIG. 3D shows pharmacological response of a non-responding clone in the presence of an AnaSpec membrane potential dye and the protein trafficking corrector (15-25 μM, same as in 3A, 3B, 3C) when challenged either by an agonist cocktail of forskolin (30 μM)+IBMX (100 μM) (black trace) or DMSO+Buffer (grey trace). FIG. 3E shows pharmacological response of a stable CFTR-ΔF508 expressing clone in the presence of an AnaSpec membrane potential dye and without the protein trafficking corrector when challenged either by an agonist cocktail of forskolin (30 μM)+IBMX (100 μM) (black trace) or DMSO+Buffer (grey trace). FIG. 3F shows pharmacological response of a non-responding clone in the presence of an AnaSpec membrane potential dye and without the protein trafficking corrector when challenged either by an agonist cocktail of forskolin (30 μM)+IBMX (100 μM) (black trace) or DMSO+Buffer (grey trace).

### DETAILED DISCLOSURE

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. The materials, methods, and examples are illustrative only and not intended to be limiting.

In order that the present invention may be more readily understood, certain terms are first defined. Additional definitions are set forth throughout the detailed description.

The term “stable” or “stably expressing” is meant to distinguish the cells and cell lines of the invention from cells with transient expression as the terms “stable expression” and “transient expression” would be understood by a person of skill in the art.

The term “cell line” or “clonal cell line” refers to a population of cells that are all progeny of a single original cell. As used herein, cell lines are maintained in vitro in cell culture and may be frozen in aliquots to establish banks of clonal cells.

The term “stringent conditions” or “stringent hybridization conditions” describe temperature and salt conditions for hybridizing one or more nucleic acid probes to a nucleic acid sample and washing off probes that have not bound specifically to target nucleic acids in the sample. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. An example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. Stringent conditions include hybridization in 0.5M sodium phosphate, 7% SDS at 65° C., followed by at least one wash at 0.2×SSC, 1% SDS at 65° C.

The phrase “percent identical” or “percent identity” in connection with amino acid and/or nucleic acid sequences refers to the similarity between at least two different sequences. This percent identity can be determined by standard alignment algorithms, for example, the Basic Local Alignment Tool (BLAST) described by Altshul et al. ((1990) J. Mol. Biol., 215: 403-410); the algorithm of Needleman et al. ((1970) J. Mol. Biol., 48: 444-453); or the algorithm of Meyers et al. ((1988) Comput. Appl. Biosci., 4: 11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) that has been incorporated into the ALIGN program (version 2.0), using a PAM120 weight residue table, a gap length penalty of 12 and a gap penalty of 4. The percent identity is usually calculated by comparing sequences of similar length. Protein analysis software matches similar sequences using measures of similarity assigned to various substitutions, deletions and other modifications, including conservative amino acid substitutions. For instance, the GCG Wisconsin Package (Accelrys, Inc.) contains programs such as “Gap” and “Bestfit” that can be used with default parameters to determine sequence identity between closely related polypeptides, such as homologous polypeptides from different species of organisms or between a wild type protein and a mutant thereof. See, e.g., GCG Version 6.1. Polypeptide sequences also can be compared using FASTA using default or recommended parameters. A program in GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). The length of polypeptide sequences compared for identity will generally be at least about 16 amino acid residues, usually at least about 20 residues, more usually at least about 24 residues, typically at least about 28 residues, and preferably more than about 35 residues. The length of a DNA sequence compared for identity will generally be at least about 48 nucleic acid residues, usually at least about 60 nucleic acid residues, more usually at least about 72 nucleic acid residues, typically at least about 84 nucleic acid residues, and preferably more than about 105 nucleic acid residues.

The phrase “substantially as set out,” “substantially identical” or “substantially homologous” in connection with an amino acid nucleotide sequence means that the relevant amino acid or nucleotide sequence will be identical to or have differences (through conserved amino acid substitutions) in comparison to the sequences that are set out. Insubstantial differences include minor amino acid changes, such as 1 or 2 substitutions in a 50 amino acid sequence of a specified region. Insubstantial differences may have deleterious effect.

The terms “potentiator”, “corrector”, “agonist” or “activator” refer to a compound or substance that activates a biological function of CFTR, e.g., increases ion conductance via CFTR. As used herein, a potentiator, corrector or activator may act upon a CFTR or upon a specific subset of different forms (e.g., mutant forms) of CFTR.

The terms “inhibitor”, “antagonist” or “blocker” refers to a compound or substance that decreases a biological function of CFTR, e.g., decreases ion conductance via CFTR. As used herein, an inhibitor or blocker may act upon a CFTR or upon a specific subset of different forms (e.g., mutant forms) of CFTR.

The term “modulator” refers to a compound or substance that alters a structure, conformation, biochemical or biophysical property or functionality of a CFTR either positively or negatively. The modulator can be a CFTR agonist (potentiator, corrector, or activator) or antagonist (inhibitor or blocker), including partial agonists or antagonists, selective agonists or antagonists and inverse agonists, and can be an allosteric modulator. A substance or compound is a modulator even if its modulating activity changes under different conditions or concentrations or with respect to different forms (e.g., mutant forms) of CFTR. As used herein, a modulator may affect the ion conductance of a CFTR, the response of a CFTR to another regulatory compound, or the selectivity of a CFTR. A modulator may also change the ability of another modulator to affect the function of a CFTR. A modulator may act upon all or upon a specific subset of different forms (e.g., mutant forms) of CFTR. Modulators include, but are not limited to, potentiators, correctors, activators, inhibitors, agonists, antagonists, and blockers. Modulators also include protein trafficking correctors.

The phrase “functional CFTR” refers to a CFTR that responds to a known activator (such as apigenin, forskolin or IBMX—[3-isobutyl-1-methylxanthine]) or a known inhibitor (such as chromanol 293B, glibenclamide, lonidamine, NPPB—[5-nitro-2-(3-phenylpropylamino)benzoic acid], DPC—[diphenylamine-2-carboxylate] or niflumic acid) or other known modulators (such as 9-AC—[anthracene-9-carboxylic acid], or chlorotoxin) in substantially the same way as CFTR in a cell that normally expresses CFTR without engineering. CFTR behavior can be determined by, for example, physiological activities, and pharmacological responses. Physiological activities include, but are not limited to, chloride ion conductance. Pharmacological responses include, but are not limited to, activation by forskolin alone, or a mixture of forskolin, apigenin and IBMX [3-isobutyl-1-methylxanthine].

A “heterologous” or “introduced” CFTR protein means that the CFTR protein is encoded by a polynucleotide introduced into a host cell.

This invention relates to novel cells and cell lines that have been engineered to express CFTR. In some embodiments, the novel cells or cell lines of the invention express a functional, wild type CFTR (e.g., SEQ ID NO: 2). In some embodiments, the CFTR is a mutant CFTR (e.g., CFTR ΔF508; SEQ ID NO: 7). Illustrative CFTR mutants are set forth in Tables 1 and 2 (These tables are compiled based on mutation information obtained from a database developed by the Cystic Fibrosis Genetic Analysis Consortium available at www.genet.sickkids.on.ca/cftr/Home). According to the invention, the CFTR can be from any mammal, including rat, mouse, rabbit, goat, dog, cow, pig, or primate (e.g., human). In some embodiments, the novel cells or cell lines express an introduced functional CFTR (e.g., CFTR encoded by a transgene). In some embodiments, the novel cells or cell lines express a naturally-occurring CFTR, encoded by an endogenous CFTR gene that has been activated by gene activation technology. In preferred embodiments, the cells and cell lines stably express CFTR. The CFTR-expressing cells and cell lines of the invention have enhanced properties compared to cells and cell lines made by conventional methods. For example, the CFTR cells and cell lines have enhanced stability of expression (even when maintained in culture without selective pressure such as antibiotics) and possess high Z′ values in cell-based assays. The cells and cell lines of the invention provide detectable signal-to-noise signals, e.g., a signal-to-noise signal greater than 1:1. The cells and cell lines of the invention provide reliable readouts when used in high-throughput assays such as membrane potential assays, producing results that can match those from assays that are considered gold-standard in the field but too labor-intensive to become high-throughput (e.g., electrophysiology assays). In certain embodiments, the CFTR does not comprise a polypeptide tag.

TABLE 1
CFTR Mutants

| | | Location | |
|---|---|---|---|
| | | of | |
| Name | Nucleotide Change | Mutation* | Consequence |
| 1001 + 11C/T | C or T at 1001 + 11 | intron 6b | sequence variation |
| 1001 + 12C/T | C or T at 1001 + 12 | intron 6b | sequence variation |
| 1001 + 3A > T | A to T at 1001 + 3 | intron 6b | Alternative splicing and |
| | | | complete skipping of exon 6b |
| 1001 + 4A->C + | | intron 6b | splicing |
| 993delCTTAA | | | |
| 1002 − 2A > G | A to G at 1002 − 2 | 6b | mRNA splicing defect |
| 1002 − 3T->G | T to G at 1002 − 3 | intron 6b | mRNA splicing defect |
| 1002 − 56C/G | C or G at 1002 − 56 | intron 6b | sequence variation |
| 1002 − 7delTTT | Deletion of TTT beginning at | intron 6b | Interference with splicing |
| | 1002 − 7 | | |
| 1013delAA | deletion of AA from 1013 | 7 | frameshift |
| −102T->A | T to A at −102 | promotor | regulatory mutation |
| 1047C/T | C or T at 1047 | 7 | sequence variation |
| 1058delC | deletion of C at 1058 | 7 | frameshift |
| 1078delT | deletion of T at 1078 | 7 | frameshift |
| 107 G/A | G to A at 107 | 1 | sequence variation |
| 1086G/A | G or A at 1086 | 7 | Sequence variation |
| 1092A/G | A or G at 1092 | 7 | sequence variation |
| 1098G/A | G or A at 1098 | 7 | sequence variation (Val at 322 |
| | | | no change) |
| 1104(C/G) | C or G at 1104 | 7 | sequence variation |
| 1112delT | deletion of T at 1112 | 7 | frameshift |
| 1119delA | deletion of A at 1119 | 7 | frameshift |
| 1138insG | insertion of G after 1138 | 7 | frameshift |
| 1150delA | deletion of A at 1150 | 7 | frameshift |
| 1150insTC | Insertion of TC at 1150 | 7 | Frameshift |
| 1151ins12 | tandem duplication of 12 bp from | 7 | Insertion-duplication of 4 |
| | position 1140 to position 1151 | | amino acids within the M6 |
| | | | domain (transmembrane |
| | | | domain) |
| 1154insTC | insertion of TC after 1154 | 7 | frameshift |
| 1161delC | deletion of C at 1161 | 7 | frameshift |
| 1161insG | insertion of G after 1161 | 7 | frameshift |
| 1164 T/A | T to A at 1164 | 7 | sequence variation |
| 1185delTC | Deletion of TC at 1185 | 7 | Frameshift |
| 1199delG | deletion of G at 1199 | 7 | frameshift |
| 120del23 | Deletion of 23 bp from nucleotide + | promotor, 1 | This mutation abolishes the |
| | 120 of exon 1 promoter, to | | initiation codon at position |
| | nucleotide 142 (the first nucleotide | | 133. The next possible |
| | of codon 4) | | initiation codon is located at |
| | | | intron 1 position 185 + 63. |
| 1213delT | deletion of T at 1213 | 7 | frameshift |
| 1215delG | deletion of G at 1215 | 7 | frameshift |
| 1221delCT | deletion of CT from 1221 | 7 | frameshift |
| 1233A/T | A or T at 1233 | 7 | Sequence variation |
| 1243ins6 | insertion of ACAAAA after 1243 | 7 | insertion of Asp and Lys after |
| | | | Lys370 |
| 1248 + 17C->T | C or T at 1248 + 17 | intron 7 | sequence variation |
| 1248 + 1G->A | G to A at 1248 + 1 | intron 7 | mRNA splicing defect |
| 1248 + 1G->C | G to C at 1248 + 1 | intron 7 | Splicing |
| 1248 + 31 A/C | 1248 + 31 A > C | intron 7 | sequence variation |
| 1248 + 52T/C | T or C at 1248 + 52 | intron 7 | sequence variation |
| 1249 − 27delTA | deletion of TA at 1249 − 27 | intron 7 | mRNA splicing defect |
| 1249 − 30delAT | deletion of AT from 1249 − 30 | intron 7 | mRNA splicing defect |
| 1249 − 31A->G | 1249 − 31 A > G | intron 7 | mRNA splicing defect |
| 1249 − 5A->G | A to G at 1249 | intron 7 | mRNA splicing defect |
| 1249 − 82C/T | C or T at 1249 − 82 | intron 7 | sequence variation |
| 124del23bp | delete 23 bp from 124 to 146 | 1 | |
| 1259insA | insertion of A after 1259 | 8 | frameshift |
| 125G/C | G or C at 125 | 1 | sequence variation |
| 1283delA | deletion of A at 1283 | 8 | frameshift |
| 1288insTA | Insertion of TA at 1285 Or | 8 | Frameshift |
| | Insertion of AT at 1284 | | |
| 1289insTA | Insertion of TA at 1289 | 8 | Frameshift |
| 1291delTT | delete TT from 1291 | 8 | Frame shift |
| 1294del7 | deletion of 7 bp from 1294 | 8 | frameshift |
| 1296G/T | G to T at 1296 | 8 | sequence variation (Thr at 388 |
| | | | no change) |
| 129G/C | G or C at 129 | 1 | sequence variation |
| 1309delG | deletion of G at 1309 | 8 | frameshift |
| 1323insA | insertion of A after 1323 | 8 | frameshift |
| 1341 + 18A->C | A to C at 1341 + 18 | intron 8 | mRNA splicing defect |
| 1341 + 1G->A | G to A at 1341 + 1 | intron 8 | mRNA splicing defect |
| 1341 + 28C > T | C > T at 1341 + 28 | intron 8 | polymorphism |
| 1341 + 28C/T | C or T at 1341 + 28 | intron 8 | sequence variation |
| 1341 + 6 A->G | A to G at 1341 + 6 | | mRNA splicing defect |
| 1341 + 6 A->G | A to G at 1341 + 6 | intron 8 | mRNA splicing defect |
| 1341 + 79 C/T | 1341 + 79 C->T | intron 8 | sequence variation |
| 1341G->A | G to A at 1341 | 8 | sequence variation |
| 1342 − 11TTT->G | TTT to G at 1342 − 11 | intron 8 | mRNA splicing defect |
| 1342 − 12(GT)n | variable number of copies (8-10x) | intron 8 | sequence variation |
| | at around 1342 − 12 to − 35 | | |
| 1342 − 13G/T | G or T at 1342 − 13 | intron 8 | sequence variation |
| 1342 − 1delG | Deletion of G at 1342 − 1 | Intron 8 | Frameshift |
| 1342 − 1G->C | G to C at 1342 − 1 | intron 8 | mRNA splicing defect |
| 1342 − 265(GT)n | variable number of copies at | intron 8 | sequence variation (greater |
| | around 1342 − 265 to − 310 | | than 8 alleles) |
| 1342 − 2A->C | A to C at 1342 − 2 | intron 8 | mRNA splicing defect |
| 1342 − 2delAG | deletion of AG from 1342 − 2 | intron 8 | mRNA splicing defect |
| 135del120ins300 | | 1 | |
| 1366delG | deletion of G at 1366 | 9 | frameshift |
| 1367del5 | deletion of CAAAA at 1367 | 9 | frameshift |
| 1367delC | deletion of C at 1367 | 9 | frameshift |
| 1429del7bp | deletion of 17bp from 1429 | 19 | stop codon at amino acid 441 |
| 1460delAT | deletion of AT from 1460 | 9 | frameshift |
| 1461ins4 | insertion of AGAT after 1461 | 9 | frameshift |
| 1461 T/C | T to C at 1461 | 9 | sequence variation |
| 1471delA | deletion of A at 1471 | 9 | frameshift |
| 1491 − 1500del | Deletion between 1491 to 1500 | 9 | Large in/del |
| 1497delGG | deletion of GG at 1497 | 9 | frameshift |
| 1504delG | deletion of G at 1504 | 9 | frameshift |
| 1524 + 1G->A | G to A at 1524 + 1 | intron 9 | splice mutation |
| 1524 + 60 insA | Ins A at 1524 + 60 | intron 9 | sequence variation |
| 1524 + 68 G/A | 1524 + 68 G > A | intron 9 | sequence variation |
| 1524 + 6insC | insertion of C after 1524 + 6, with | intron 9 | mRNA splicing defect |
| | G to A at 1524 + 12 | | |
| 1525 − 18G/A | G or A at 1525 − 18 | intron 9 | sequence variation or mRNA |
| | | | splicing defect |
| 1525 − 1G->A | G to A at 1525 − 1 | intron 9 | mRNA splicing defect |
| 1525 − 2A->G | A to G at 1525 − 2 | intron 9 | Splicing |
| 1525 − 47T->G | 1525 − 47T > G | Intron 9 | Sequence Variation |
| 1525 − 60G/A | G or A at 1525 − 60 | intron 9 | sequence variation |
| 1525 − 61A/G | A or G at 1525 − 61 | intron 9 | sequence variation |
| 1531C/T (L467F) | C or T at 1531 | 10 | sequence variation |
| 1540del10 | deletion of 10bp after 1540 | 10 | frameshift |
| 1548delG | deletion of G from 1548 − 1550 | 10 | frameshift |
| 1565 del CA | deletion of CA from 1565 | 10 | frameshift |
| 156G/A | G or A at 156 | 1 | sequence variation |
| 1571delG | deletion of G at 1571 | 10 | frameshift |
| 1572T/C | T or C at 1572 | 10 | sequence variation |
| 1576insT | insertion of T at 1576 | 10 | framshift |
| 1601delTC | deletion of TC from 1601 or CT | 10 | frameshift |
| | from 1602 | | |
| 1609delCA | deletion of CA from 1609 | 10 | frameshift |
| 1612delTT | deletion of TT from 1612 | 10 | frameshift |
| 163G/A | G or A at 163 | 1 | sequence variation |
| 1650C/G | C to G at 1650 | 10 | Ile to Met at 506; sequence |
| | | | variation |
| 1651A/G | A or G at 1651 | 10 | sequence variation |
| 1653C/T | C to T at 1653 | 10 | NO AMINOACID CHANGE |
| 1660delG | Deletion of G at 1660 | 10 | frameshift |
| 1677delTA | deletion of TA from 1677 | 10 | frameshift |
| 1693A->C | A to C at 1693 | 10 | Ile to Leu at 521 (sequence |
| | | | variation) |
| 1706del17 | deletion of 17 bp from 1706 | 10 | deletion of splice site |
| 1713A/G | A or G at 1713 | 10 | sequence variation |
| 1716 + 12T/C | T or C at 1716 + 12 | intron 10 | sequence variation |
| 1716 + 13G/T | G or T at 1716 + 13 | intron 10 | sequence variation |
| 1716 + 1G->A | G to A at 1716 + 1 | intron 10 | mRNA splicing defect |
| 1716 + 1G->T | 1716 + 1 G > T | intron 10 | mRNA splicing defect |
| 1716 + 2T->C | T to C at 1716 + 2 | intron 10 | mRNA splicing defect |
| 1716 + 4 A->T | 1716 + 4 A > T | intron 10 | mRNA splicing defect |
| 1716 + 63ins11nt | insertion of 11 nucleotides after | intron 10 | sequence variation |
| | 1716 + 63 | | |
| 1716 + 64A/C | A or C at 1716 + 64 | intron 10 | sequence variation |
| 1716 + 77A/G | A or G at 1716 + 77 | intron 10 | sequence variation |
| 1716 + 85C/T | C or T at 1716 + 85 | intron 10 | sequence variation |
| 1716G/A | G or A at 1716 | 10 | sequence variation |
| 1717 − 19T/C | T or C at 1717 − 19 | intron 10 | sequence variation |
| 1717 − 1G->A | G to A at 1717 − 1 | intron 10 | mRNA splicing defect |
| 1717 − 2A->G | A to G at 1717 − 2 | intron 10 | mRNA splicing defect |
| 1717 − 3T->G | T to G at 1717 − 3 | intron 10 | mRNA splicing defect |
| 1717 − 8G->A | G to A at 1717 − 8 | intron 10 | mRNA splicing defect |
| 1717 − 9T->A | T to A at 1717 − 9 | intron 10 | mRNA splicing mutation |
| 1742delAC | deletion of AC from 1742 | 11 | frameshift |
| 1749insTA | insertion of TA at 1749 | 11 | frameshift resulting in |
| | | | premature termination at 540 |
| 174delA | deletion of A between 172 − 174 | 1 | frameshift |
| 175delC | deletion of C at 175 | 1 | frameshift |
| 175insT | insertion of T after 175 | 1 | frameshift |
| 1764T/G | T or G at 1764 | 11 | sequence variation |
| 1767del6 | delete 6 nucleotide from 1767 | 11 | In frame in/del |
| 1773A/T | A or T at 1773 | 11 | sequence variation |
| 1774delCT | deletion of CT from 1774 | 11 | frameshift |
| 1782delA | deletion of A at 1782 | 11 | frameshift |
| 1784delG | deletion of G at 1784 | 11 | frameshift |
| 1787delA | deletion of A at position 1787 or | 11 | frameshift, stop codon at 558 |
| | 1788 | | |
| 1802delC | deletion of C at 1802 | 11 | frameshift |
| 1806delA | deletion of A at 1806 | 11 | frameshift |
| 1811 + 11A->G | A to G at 1811 + 11 | intron 11 | Splicing |
| 1811 + 1650 T > A | 1811 + 1650 T > A | intron 11 | Sequence variation |
| 1811 + 1.6kbA->G | A to G at 1811 + 1.2kb | intron 11 | creation of splice donor site |
| 1811 + 16T->C | 1811 + 16 T > C | intron 11 | This mutation may lead to an |
| | | | alternative splicing, with the |
| | | | donor splice site located at |
| | | | nucleotide + 18. This |
| | | | alternative splice site with the |
| | | | mutation at +16 has a higher |
| | | | PCU than the previously |
| | | | described mutation |
| | | | 1811 + 18G->A. |
| 1811 + 18G->A | G to A at 1811 + 18 | intron 11 | mRNA splicing defect |
| 1811 + 1 G > A | G to A at 1811 + 1 | intron 11 | Splicing defect |
| 1811 + 1G->C | G to C at 1811 + 1 | intron 11 | mRNA splicing defect |
| 1811 + 24G->A | G to A at 1811 + 24 | Intron 11 | mRNA splicing defect |
| 1811 + 34 G > A | G to A at 1811 + 34 | intron 11 | mRNA splicing defect |
| 1811 + 5A->G | 1811 + 5 A > G | intron 11 | mRNA splicing defect |
| 1812 − 108T/C | T or C at 1812 − 108 | intron 11 | sequence variation |
| 1812 − 136T/C | T or C at 1812 − 136 | intron 11 | sequence variation |
| 1812 − 1G->A | G to A at 1812 − 1 | intron 11 | mRNA splicing defect |
| 1812 − 26T->C | T to C at 1812 − 26 | intron 11 | splicing mutation |
| 1812 − 59T/G | T or G at 1812 − 59 | intron 11 | sequence variation |
| 1812 − 5 T->A | 1812 − 5 T > A | intron 11 | splicing mutation |
| 1812 − 99 T->C | C to T at 1812 − 99 | Intron 11 | Sequence Variation |
| 1813insC | insertion of C after 1813 (or 1814) | 12 | frameshift |
| 182delT | deletion of T at 182 | 1 | frameshift |
| 1833delT | deletion of T at 1833 | 12 | frameshift |
| 1845delAG/1846delGA | deletion of AG at 1845 or GA at | 12 | frameshift |
| | 1846 | | |
| 185 + 1G->T | G to T at 185 + 1 | intron 1 | mRNA splicing defect |
| 185 + 45A->G | A to G at 185 + 45 | intron 1 | sequence variation |
| 185 + 4A->T | A to T at 185 + 4 | intron 1 | mRNA splicing defect |
| | | | (CBAVD) |
| 186 − 13C->G | C to G at 186 − 13 | intron 1 | mRNA splicing defect |
| 1870delG | deletion of G at 1870 | 12 | frameshift |
| 1874insT | insertion of T between 1871 and | 12 | frameshift |
| | 1874 | | |
| 1898 + 152T/A | T or A at 1898 + 152 | intron 12 | sequence variation |
| 1898 + 1G->A | G to A at 1898 + 1 | intron 12 | mRNA splicing defect |
| 1898 + 1G->C | G to C at 1898 + 1 | intron 12 | mRNA splicing defect |
| 1898 + 1G->T | G to T at 1898 + 1 | intron 12 | mRNA splicing defect |
| 1898 + 30G/A | G or A at 1898 + 30 | intron 12 | sequence variation |
| 1898 + 3A->C | A to C at 1898 + 3 | intron 12 | mRNA splicing defect |
| 1898 + 3A->G | A to G at 1898 + 3 | intron 12 | mRNA splicing defect |
| 1898 + 5G->A | G to A at 1898 + 5 | intron 12 | mRNA splicing defect |
| 1898 + 5G->T | G to T at 1898 + 5 | intron 12 | mRNA splicing defect |
| 1898 + 73T->G | T to G at 1898 + 73 | intron 12 | mRNA splicing defect |
| 1918delGC | deletion of GC from 1918 | 13 | frameshift |
| 1924del7 | deletion of 7 bp (AAACTA) from | 13 | frameshift |
| | 1924 | | |
| 1932delG | Deletion of G at nucleotide 1932 | 13 | Frameshift a premature stop |
| | | | codon appears 10 codons |
| | | | further. |
| 1949del84 | deletion of 84 bp from 1949 | 13 | deletion of 28 a.a. (Met607 to |
| | | | Gln634) |
| 2003del8 | Deletion of GCTATTTT from 2003 | 13 | Frameshift |
| 2043delG | deletion of G at 2043 | 13 | frameshift |
| 2051delTT | deletion of TT from 2051 | 13 | frameshift |
| 2055del9->A | deletion of 9 bp CTCAAAACT to A | 13 | frameshift |
| | at 2055 | | |
| 2064C/G | C or G at 2064 | 13 | sequence variation (Leu at |
| | | | 644 no change) |
| 2082C/T | C or T at 2082 | 13 | sequence variation (no |
| | | | change Phe at 650) |
| 2092A/G | A or G at 2092 | 13 | sequence variation |
| 2104insA + 2109 − | insertion of A at 2104, deletion of | 13 | |
| 2118del10 | 10bp at 2109 | | |
| 2105 − | Deletion of 13 bp and insertion of | 13 | Frameshift |
| 2117del13insAGAAA | AGAAA at 2105 − 2117 | | |
| 2108delA | deletion of A at 2108 | 13 | frameshift |
| 2113delA | deletion of A at 2113 | 13 | frameshift |
| 2116delCTAA | deletion of CTAA at 2116 | 13 | frameshift |
| 2118del4 | deletion of AACT from 2118 | 13 | frameshift |
| 211delG | deletion of G at 211 | 2 | frameshift |
| 2141insA | insertion of A after 2141 | 13 | frameshift |
| 2143delT | deletion of T at 2143 | 13 | frameshift |
| 2176insC | insertion of C after 2176 | 13 | frameshift |
| 2183AA->G | A to G at 2183 and deletion of A at | 13 | frameshift |
| | 2184 | | |
| 2183delAA | deletion of AA at 2183 | 13 | frameshift |
| 2184A/G | A to G at 2184 | 13 | no change |
| 2184delA | deletion of A at 2184 | 13 | frameshift |
| 2184insA | insertion of A after 2184 | 13 | frameshift |
| 2185insC | insertion of C at 2185 | 13 | frameshift |
| 2193ins4 | Insertion of 4T at 2193 | 13 | Frameshift |
| 2215insG | insertion of G at 2215 | 13 | frameshift |
| 2221insA | insertion of A at 2221 | 13 | Frameshift a premature stop |
| | | | codon appears 33 codons |
| | | | further |
| 2238C/G | C or G at 2238 | 13 | sequence variation |
| 223C/T | C or T at 223 | 2 | sequence variation |
| 2289 − 2295del7bpinsGT | Deletion of 7 bp and insertion of | 13 | Frameshift |
| | GT at 2289 − 2295 | | |
| 2307insA | insertion of A after 2307 | 13 | frameshift |
| 232del18 | Deletion of 18 bp from 232 | 2 | Deletion of 6 aa from Leu34 to |
| | | | Gln39 |
| 2335delA | deletion of A at 2335 | 13 | frameshift |
| 2347delG | deletion of G at 2347 | 13 | frameshift |
| 2372del8 | deletion of 8 bp from 2372 | 13 | frameshift |
| 2377C/T | C or T at 2377 | 13 | sequence variation (no |
| | | | change for Leu at 749) |
| 237insA | insertion of A after 237 | 2 | frameshift |
| 2380_2387del | Deletion of 8 bp from 2380 | 13 | Frameshift |
| 2391 C/T | 2391 C > T | 13 | Polymorphism |
| 2406delCC | deletion of CC at 2406 | 13 | Frameshift |
| 2409delC | Deletion of C at 2409 | 13 | Frameshift |
| 2412G/A | G to A at 2412 | 13 | Sequence variation |
| 2418GG > T | G to T at 2418 | 13 | missense |
| 241delAT | deletion of AT from 241 | 2 | frameshift |
| 2423delG | deletion of G at 2423 | 13 | frameshift |
| 244delTA | deletion of TA from 244 | 2 | frameshift |
| 2456delAC | deletion of AC at 2456 | 13 | frameshift |
| 2493ins8 | insertion of 8bp after 2493 | 13 | frameshift |
| 2512delG | Deletion of G at 2512 | 13 | Frameshift |
| 2522insC | insertion of C after 2522 | 13 | frameshift |
| 2553A/G | A or G at 2553 | 13 | sequence variation |
| 2556insAT | insertion of AT after 2556 | 13 | frameshift |
| 2566insT | insertion of T after 2566 | 13 | frameshift |
| 2585delT | deletion of T at 2585 | 13 | stop codon at amino acid 820 |
| 2603delT | deletion of T at 2603/4 | 13 | frameshift |
| 2622 + 14G/A | G or A at 2622 + 14 | intron 13 | sequence variation |
| 2622 + 1G->A | G to A at 2622 + 1 | intron 13 | mRNA splicing defect |
| 2622 + 1G->T | G to T at 2622 + 1 | intron 13 | splice mutation |
| 2622 + 2del6 | deletion of TAGGTA from 2622 + 2 | intron 13 | mRNA splicing defect |
| 2622 + 2T > C | T to C at 2622 + 2 | intron 13 | mRNA splicing defect |
| 2623 − 11 C->T | 2623 − 11 C > T | intron 13 | Polymorphism |
| 2623 − 23A->G | 2623 − 23 A > G | intron 13 | mRNA splicing defect |
| 2623 − 2A->G | A to G at 2623 − 2 | intron 23 | Splicing |
| 2634delT | Deletion of T at 2634 | 14a | frameshift |
| 2634insT | insertion of T after 2634 | 14a | frameshift |
| 263A/T | A or T at 263 | 2 | sequence variation |
| 2640delT | deletion of T at 2640 | 14a | frameshift |
| 2691T/C | T or C at 2691 | 14a | sequence variation |
| 2694delT | deletion of T at 2694 | 14a | frameshift |
| 2694T/C | T or C at 2694 | 14a | sequence variation |
| 2694T/G | T or G at 2694 | 14a | sequence variation |
| 2703G/A | G or A at 2703 | 14a | sequence variation (Lys at 857 |
| | | | no change) |
| 2711delT | deletion of T at 2711 | 14a | frameshift |
| 2721del11 | deletion of 11 bp from 2721 | 14a | frameshift |
| 2723delTT | deletion of TT from 2723 | 14a | frameshift |
| 2732insA | insertion of A at 2732 | 14a | frameshift |
| 2734G->AT | Deletion of G at 2734 with | 14a | frameshift |
| | insertion of AT | | |
| 2736G/A | G or A at 2736 | 14a | sequence variation |
| 2747delC | Deletion of C at nucleotide 2747 | 14a | Frameshift a premature stop |
| | | | codon appears 34 codons |
| | | | further |
| 2751 + 2T->A | T to A at 2751 + 2 | intron | mRNA splicing defect |
| | | 14a | |
| 2751 + 3A->G | A to G at 2751 + 3 | intron | mRNA splicing defect |
| | | 14a | (CBAVD) |
| 2751G->A | G to A at 2751 | 14a | mRNA splicing defect |
| 2752 − 15C/G | C or G at 2752 − 15 | intron | sequence variation |
| | | 14a | |
| 2752 − 17G/A | G to A at 2752 − 17 | intron | sequence variation |
| | | 14a | |
| 2752 − 1G->C | G to C at 2752 − 1 | intron | splice mutation |
| | | 14a | |
| 2752 − 1G->T | G to T at 2752 − 1 | intron | mRNA splicing defect |
| | | 14a | |
| 2752 − 22A/G | A or G at 2752 − 22 | intron | sequence variation |
| | | 14a | |
| 2752 − 26A->G | A to G at 2752 − 26 | intron | mRNA splicing defect |
| | | 14a | |
| 2752 − 2A > G | A to G at 2752 − 2 | Intron | mRNA splicing defect |
| | | 14a | |
| 2752 − 674_3499 + | 2752 − 674_3499 + 198del9855bp | 14b, 15, | Large deletion removing |
| 198del9855 | | 16, 17a, | exons 14b to 17b. Frameshift |
| | | 17b | |
| 2752 − 6T->C | T to C at 2752 − 6 | intron | Splicing |
| | | 14a | |
| 2752 − 97C->T | C to T at 2752 − 97 | intron | Splicing |
| | | 14a | |
| 2766del8 | deletion of 8 bp from 2766 | 14b | frameshift |
| 2787del16 | Deletion of 16 nucleotides from | 14b, | Splicing mutation. |
| | 2787 | intron | |
| | | 14b | |
| 2789 + 2insA | insertion of A after 2789 + 2 | intron | mRNA splicing defect (CAVD) |
| | | 14b | |
| 2789 + 32T/C | T or C at 2789 + 32 | intron | sequence variation |
| | | 14b | |
| 2789 + 3delG | deletion of G at 2789 + 3 | intron | mRNA splicing defect |
| | | 14b | |
| 2789 + 5G->A | G to A at 2789 + 5 | intron | mRNA splicing defect |
| | | 14b | |
| 2790 − 108G/C | G or C at 2790 − 108 | intron | sequence variation |
| | | 14b | |
| 2790 − 1G->C | G to C at 2790 − 1 | intron | mRNA splicing defect |
| | | 14b | |
| 2790 − 1G->T | G to T at 2790 − 1 | intron | mRNA splicing defect |
| | | 14b | |
| 2790 − 21G/A | G or A at 2790 − 21 | intron | sequence variation |
| | | 14b | |
| 2790 − 2A->G | A to G at 2790 − 2 | intron | mRNA splicing defect |
| | | 14b | |
| 279A/G | A to G at 279 | 2 | No change (Leu at 49) |
| 2811G/T | G or T at 2811 | 15 | sequence variation |
| 2819del4bpins13bp | delete 4bp(CTCA) at 2819, insert | 15 | Thr to Met at 896, His to Ser |
| | 13 bp (TGAGTACTATGAG (SEQ | | at 897, insertion of Thr, Met |
| | ID NO: 10)) at 2819 | | and Ser after 897 |
| 2839T/C | T or C at 2839 | 15 | sequence variation |
| 2844A/T | A or T at 2844 | 15 | sequence variation (Ala at 904 |
| | | | no change) |
| 284delA | deletion of A at 284 | 2 | frameshift |
| 2851A/G | A or G at 2851 | 15 | Ile or Val at 907 |
| 2856C/T | C or T at 2856 | 15 | sequence variation (Thr at 908 |
| | | | no change) |
| 2858G/T | G or T at 2858 | 15 | sequence variation |
| 2868 G/A | G to A at 2868 | 15 | sequence variation |
| 2869insG | insertion of G after 2869 | 15 | frameshift |
| 2896insAG | insertion of AG after 2896 | 15 | frameshift |
| 2901C/T | C or T at 2901 | 15 | sequence variation |
| 2907delTT | deletion of TT from 2907 | 15 | frameshift |
| 2909delT | deletion of T at 2909 | 15 | frameshift |
| 2940A/G | A or G at 2940 | 15 | sequence variation |
| 2942insT | insertion of T at 2942 | 15 | frameshift resulting in |
| | | | premature termination at |
| | | | codon 974 |
| 2948AT->C | AT to C at 2948 | 15 | frameshift resulting in |
| | | | premature termination at 2953 |
| 295ins8 | insertion of ATTGGAAA after 295 | 2 | frameshift |
| 296 + 128G/C | G or C at 296 + 128 | intron 2 | sequence variation |
| 296 + 12T->C | T to C at 296 + 12 | intron 2 | mRNA splicing defect |
| 296 + 1G->A | G to A at 296 + 1 | intron 2 | splicing |
| 296 + 1G->C | G to C at 296 + 1 | intron 2 | mRNA splicing defect |
| 296 + 1G->T | G to T at 296 + 1 | intron 2 | missense; mRNA splicing |
| | | | defect |
| 296 + 28A->G | A to G at 296 + 28 | intron 2 | mRNA splicing |
| 296 + 2T->A | T to A at 296 + 2 | intron 2 | mRNA splicing Defect |
| 296 + 2T->C | T to C at 296 + 2 | intron 2 | mRNA splicing defect |
| 296 + 2T->G | T to G at 296 + 2 | intron 2 | mRNA splicing defect |
| 296 + 3insT | insertion of T after 296 + 3 | intron 2 | mRNA splicing defect |
| 2967G/A | G or A at 2967 | 15 | sequence variation (no |
| | | | change for Ser at 945) |
| 296 + 9A->T | A to T at 296 + 9 | intron 2 | mRNA splicing defect |
| 297 − 10T->G | T to G at 297 − 10 | intron 2 | splice mutation |
| 297 − 12insA | insertion of A at 297 − 12 | intron 2 | splice mutation |
| 297 − 28insA | insertion of A after 297 − 28 | intron 2 | mRNA splicing defect |
| 297 − 2A->G | A to G at 297 − 2 | intron 2 | mRNA splicing defect |
| 297 − 3C->A | C to A at 297 − 3 | intron 2 | mRNA splicing defect |
| 297 − 3C->T | C to T at 297 − 3 | intron 2 | mRNA splicing defect |
| 297 − 45 A->G | A to G at 297 − 45 | | Sequence variation |
| 297 − 50A/G | A or G at 297 − 50 | intron 2 | sequence variation |
| 297 − 55C/T | C to T at 297 − 55 | intron 2 | sequence variation |
| 297 − 57 G/T | 297 − 57 G > T | intron 2 | sequence variation |
| 297 − 67A/C | A or C at 297 − 67 | intron 2 | sequence variation |
| 297 − 73 A/G | 297 − 73 A > G | intron 2 | sequence variation |
| 2991del32 | deletion of 32 bp from 2991 to | 15 | frameshift |
| | 3022 | | |
| 3007delG | deletion of G at 3007 | 15 | frameshift |
| 300delA | deletion of A at 300 | 3 | frameshift |
| 3028delA | deletion of A at 3028 | 15 | frameshift |
| 3030G/A | G or A at 3030 | 15 | sequence variation |
| 3040 + 11A/T | 3040 + 11 A > T | intron 15 | Polymorphism |
| 3040 + 23T->C | T to C at 3040 + 23 | intron 15 | Splicing |
| 3040 + 2T->C | T to C at 3040 + 2 | intron 15 | mRNA splicing defect |
| 3041 − 11del7 | deletion of GTATATT at 3041 − 11 | intron 15 | mRNA splicing mutation |
| 3041 − 15T->G | T to G at 3041 − 15 | intron 15 | mRNA splicing mutation |
| 3041 − 1G->A | G to A at 3041 − 1 | intron 15 | mRNA splicing defect |
| 3041 − 4A->G | A to G at 3041 − 4 | intron 6b | splicing |
| 3041 − 51 T/G | 3041 − 51 T > G | intron 15 | sequence variation |
| 3041 − 52C/G | C or G at 3041 − 52 | intron 15 | sequence variation |
| 3041 − 71G/C | G or C at 3041 − 71 | intron 15 | sequence variation |
| 3041 − 92G/A | G or A at 3041 − 92 | intron 15 | sequence variation |
| 3041delG | deletion of G at 3041 | 16 | frameshift |
| 3056delGA | deletion of GA from 3056 | 16 | frameshift |
| 306delTAGA | deletion of TAGA from 306 | 3 | frameshift |
| 306insA | insertion of A at 306 | 3 | frameshift |
| 3079delTT | deletion of TT from 3079 | 16 | frameshift |
| 3100insA | insertion of A after 3100 | 16 | frameshift |
| 3120 + 198G->A | G to A at 3120 + 198 | intron 16 | Splicing |
| 3120 + 1G->A | G to A at 3120 + 1 | intron 16 | mRNA splicing defect |
| 3120 + 35 A->T | A to T at 3120 + 35 | Intron 16 | mRNA splicing defect |
| 3120 + 41delA | Delete A at 3120 + 41 | intron 16 | sequence variation |
| 3120 + 45A/G | A or G at 3120 + 45 | intron 16 | sequence variation |
| 3120G->A | G to A at 3120 | 16 | mRNA splicing defect |
| 3121 − 14C/A | C or A at 3121 − 14 | intron 16 | Sequence variation |
| 3121 − 1G->A | G to A at 3121 − 1 | intron 16 | mRNA splicing defect |
| 3121 − 2A->G | A to G at 3121 − 2 | intron 16 | mRNA splicing defect |
| 3121 − 2A->T | A to T at 3121 − 2 | intron 16 | mRNA splicing defect |
| 3121 − 3C->G | C to G at 3121 − 3 | intron 16 | mRNA splicing |
| 3121 − 92A12/13 | 12A or 13A at 3121 − 92 | intron 16 | sequence variation |
| 3121 − 977_3499 + | 3121 − 977_3499 + 248del2515bp | 17a, 17b | Large deletion removing |
| 248del2515 | | | exons 17a and 17b. |
| | | | Frameshift |
| 3126del4 | deletion of ATTA from 3126 | 17a | frameshift |
| 3129del4 | deletion of 4 bp from 3129 | 17a | frameshift |
| 3130del15 | delete 15 nucleotide at 3130 | 17a | In fram in/del |
| 3130delA | Deletion of A at 3130 | 17a | frameshift |
| 3131del15 | deletion of 15 bp from 3130, 3131, | 17a | deletion of Val at 1001 to Ile at |
| | or 3132 | | 1005 |
| 3132delTG | deletion of TG from 3132 | 17a | frameshift |
| 3141del9 | del AGCTATAGC from 3141 | 17a | Frameshift |
| 3152delT | delete T at 3152 | 17a | frameshift |
| 3153delT | deletion of T at 3153 | 17a | frameshift |
| 3154delG | deletion of G at 3154 | 17a | frameshift |
| 3171delC | deletion of C at 3171 | 17a | frameshift resulting in |
| | | | premature termination at 1022 |
| 3171insC | insertion of C after 3171 | 17a | frameshift |
| 3173delAC | deletion of AC from 3173 | 17a | frameshift |
| 3195del6 | deletion of AGTGAT from 3195 to | 17a | deletion of Val1022 and |
| | 3200 | | Ile1023 |
| 3196del54 | deletion of 54 bp from 3196 | 17a | deletion of 18 aa from codon |
| | | | 1022 |
| 3199del6 | deletion of AGTG from 3199 | 17a | deletion of Ile at 1023 and Val |
| | | | at 1024 |
| 3200_3204delTAGTG | Deletion of TAGTG from 3200 | 17a | Frameshift |
| 3238delA | 3238delA | 17a | frameshift |
| 3271 + 101C/G | C or G at 3271 + 101 | intron | sequence variation |
| | | 17a | |
| 3271 + 183 T to G | T to G at 3271 + 183 | intron | sequence variation |
| | | 17a | |
| 3271 + 18C/T | C or T at 3271 + 18 | intron | sequence variation |
| | | 17a | |
| 3271 + 1G->A | G to A at 3271 + 1 | intron | mRNA splicing defect |
| | | 17a | |
| 3271 + 1G > T | G to T at 3271 + 1 | Intron | mRNA splicing defect |
| | | 17a | |
| 3271 + 42A/T | A or T at 3271 + 42 | intron | sequence variation |
| | | 17a | |
| 3271 + 80A/T | A or T at 3271 + 80 | Intron | Sequence variation |
| | | 17a | |
| 3271 + 8A > G | A to G at 3271 + 8 | intron | RNA splicing defect |
| | | 17a | |
| 3271delGG | deletion of GG at 3271 | 17a | framshift for exon 17b, loss of |
| | | | splice site |
| 3272 − 11A->G | A to G at 3272 − 11 | intron | Splicing |
| | | 17a | |
| 3272 − 1G->A | G to A at 3272 − 1 | intron | mRNA splicing defect |
| | | 17a | |
| 3272 − 26A->G | A to G at 3272 − 26 | intron | mRNA splicing defect |
| | | 17a | |
| 3272 − 33A/G | A or G at 3272 − 33 | intron | sequence variation |
| | | 17a | |
| 3272 − 42 G/T | 3272 − 42 G > T | intron | sequence variation |
| | | 17a | |
| 3272 − 4A->G | A to G at 3272 − 4 | intron | mRNA splicing defect |
| | | 17a | |
| 3272 − 54del704 | deletion of 704 bp from 3272 − 54 | intron | deletion of exon 17b |
| | | 17a | |
| 3272 − 93T/C | T or C at 3272 − 93 | intron | sequence variation |
| | | 17a | |
| 3272 − 9A->T | A to T at 3272 − 9 | intron | mRNA splicing defect |
| | | 17a | |
| 3293delA | deletion of A at 3293 | 17b | frameshift |
| −329A/G | A or G at −329 upstream of the | promotor | sequence variation |
| | cap site | | |
| 3320ins5 | insertion of CTATG after 3320 | 17b | frameshift |
| 3333C/T | C or T at 3333 | 17b | sequence variation |
| 3336C/T | C or T at 3336 | 17b | sequence variation |
| 3359delCT | deletion of CT from 3359 | 17b | frameshift |
| 3384A/G | A or G at 3384 | 17b | sequence variation |
| 3396delC | deletion of C at 3396 | 17 | frameshift |
| −33G->A | G to A at −33 | promotor | promoter mutation |
| 3413del355_insTGTTAA | Partial deletion of exon 17b. It | 17b | A stop codon appears very |
| | removes 355 bp, i.e. from nt 3413 | | early in the new sequence but |
| | (in codon 1094) to 3499 + 268 in | | the consequences at the RNA |
| | intron 17b; the sequence | | level remain to be studied. |
| | “TGTTAA” is inserted at the | | |
| | breakpoints. | | |
| 3417A/T | A or T at 3417 | 17b | sequence variation |
| 3419delT | deletion of T at 3419 | 17b | frameshift |
| 3423delC | deletion of C at 3423 | 17b | frameshift |
| 3425delG | deletion of G at 3425 or 3426 | 17b | frameshift |
| 3438A/G | A or G at 3438 | 17b | Sequence variation |
| 3447delG | Deleletion of G at 3447 | 17b | Frameshift |
| 345T/C | T or C at 345 | 3 | sequence variation |
| 3471T/C | T or C at 3471 | 17b | sequence variation |
| 3477C/A | C or A at 3477 | 17b | sequence variation |
| 347delC | deletion of C at 347 | 3 | frameshift |
| 3495delA | deletion of A at 3495 | 17b | frameshift |
| 3499 + 29G/A | G or A at 3499 + 29 | Intron | Sequence variation |
| | | 17b | |
| 3499 + 2T->C | T to C at 3499 + 2 | intron | mRNA splicing defect |
| | | 17b | |
| 3499 + 37G/A | G or A at 3499 + 37 | intron | sequence variation |
| | | 17b | |
| 3499 + 3A->G | A to G at 3499 + 3 | intron | mRNA splicing defect |
| | | 17b | |
| 3499 + 45T/C | T or C at 3499 + 45 | intron | sequence variation |
| | | 17b | |
| 3499 + 6A->G | A to G at 3499 + 6 | intron | mRNA splicing defect |
| | | 17b | |
| 3499 + 7T->G | T to G at 3499 + 7 | intron | Splicing |
| | | 17b | |
| 3500 − 140A/C | A or C at 3500 − 140 | intron | sequence variation |
| | | 17b | |
| 3500 − 1 G to A | 3500 − 1 G > A | intron | mRNA splicing defect |
| | | 17b | |
| 3500 − 2A->G | A to G at 3500 − 2 | intron | mRNA splicing defect |
| | | 17b | |
| 3500 − 44G/A | G or A at 3500 − 44 | intron | sequence variation |
| | | 17b | |
| 3500 − 50 A/C | 3500 − 50 A > C | intron | sequence variation |
| | | 17b | |
| 3523A->G | A to G at 3523 | 18 | Ile to Val at 1131 |
| 3532AC->GTA | AC to GTA from 3532 | 18 | frameshift |
| 3556insAGTA | insertion of AGTA after position | 18 | frame shift |
| | 3556 | | |
| 3577delT | deletion of T at 3577 | 18 | frameshift |
| 359insT | insertion of T after 359 | 3 | frameshift |
| 3600 + 2insT | insertion of T after 3600 + 2 | intron 18 | mRNA splicing defect |
| 3600 + 2T->C | T to C at 3600 + 2 | intron 18 | sequence variation |
| 3600 + 42G/A | G or A at 3600 + 42 | intron 18 | sequence variation |
| 3600 + 5G->A | G to A at 3600 + 5 | intron 18 | mRNA splicing defect |
| 3600G->A | G to A at 3600 | 18 | mRNA splicing defect |
| 3601 − 111G/C | G or C at 3601 − 111 | intron 18 | sequence variation |
| 3601 − 17T->C | T to C at 3601 − 17 | intron 18 | mRNA splicing defect |
| 3601 − 20T->C | T to C at 3601 − 20 | intron 18 | mRNA splicing mutant |
| 3601 − 2A->G | A to G at 3601 − 2 | intron 18 | mRNA splicing defect |
| 3601 − 65C/A | C or A at 3601 − 65 | intron 18 | sequence variation |
| 360 − 365insT | Insertion of T at 360 − 365 | 3 | Frameshift |
| 360delT | deletion of T at 360 | 3 | frameshift |
| 3617delGA | Deletion of GA from 3617 | 19 | Frameshift |
| 3617G/T | G or T at 3617 | 19 | sequence variation |
| 3622insT | insertion of T after 3622 | 19 | frameshift |
| 3629delT | Deletion of T at 3629 | 19 | Frame shift |
| 3636 C/T | C to T at 3636 | 19 | sequence variation (Asp at |
| | | | 1168 no change) |
| −363C/T | C to T at −363 | promotor | promoter mutation |
| 365 − 366insT (W79fs) | insertion at 360 − 365 | 3 | Frameshift (W79fs) |
| 3659delC | deletion of C at 3659 | 19 | frameshift |
| 3662delA | deletion of A at 3662 | 19 | frameshift |
| 3667del4 | deletion of 4 bp from 3667 | 19 | frameshift |
| 3667ins4 | insertion of TCAA after 3667 | 19 | frameshift |
| 3670delA | deletion of A at 3670 | 19 | frameshift |
| 3696G/A | G to A at 3696 | 18 | No change to Ser at 1188 |
| 3724delG | deletion of G at 3724 | 19 | frameshift |
| 3726G/T | G or T at 3726 | 19 | sequence variation |
| 3732delA | deletion of A at 3732 and A to G at | 19 | frameshift and Lys to Glu at |
| | 3730 | | 1200 |
| 3737delA | deletion of A at 3737 | 19 | frameshift |
| 3750delAG | deletion of AG from 3750 | 19 | frameshift |
| 3755delG | deletion of G between 3751 and | 19 | frameshift |
| | 3755 | | |
| 3780 A/C | A to C at 3780 | 19 | sequence variation |
| 3789insA | insertion of A at 3789 | 19 | frameshift resulting in a |
| | | | premature termination at 3921 |
| 3791C/T | C or T at 3791 | 19 | sequence variation |
| 3791delC | deletion of C at 3791 | 19 | frameshift |
| 379 − 381insT | Insertion of T at 379 − 381 | 3 | Frameshift |
| 3821 − 3823del T | deletion of T at 3821 − 3823 | 19 | frameshift (Stop at 1234) |
| 3821delT | deletion of T at 3821 | 19 | frameshift |
| 3849 + 10kbC->T | C to T in a 6.2 kb EcoRI fragment | intron 19 | creation of splice acceptor site |
| | 10 kb from 19 | | |
| 3849 + 1G->A | G to A at 3849 + 1 | intron 19 | mRNA splicing defect |
| 3849 + 40A->G | A to G at 3849 + 40 | intron 19 | Splicing |
| 3849 + 45G->A | G to A at 3849 + 45 | intron 19 | Splicing |
| 3849 + 4A->G | A to G at 3849 + 4 | intron 19 | mRNA splicing defect |
| 3849 + 5G->A | G to A at 3849 + 5 | intron 19 | mRNA splicing defect |
| 3849G->A | G to A at 3849 | 19 | mRNA splicing defect |
| 3850 − 129T/C | T or C at 3850 − 129 | intron 19 | sequence variation |
| 3850 − 1G->A | G to A at 3850 − 1 | intron 19 | mRNA splicing defect |
| 3850 − 3T->G | T to G at 3850 − 3 | intron 19 | mRNA splicing defect |
| 3850 − 41C/G | 3850 − 41 C > G | intron 19 | Sequence variation |
| 3850 − 79T/C | T or C at 3850 − 79 | intron 19 | sequence variation |
| 3860ins31 | insertion of 31 bp after 3860 | 20 | frameshift |
| 3867A/G | A or G at 3867 | 20 | sequence variation |
| 3876delA | deletion of A at 3876 | 20 | frameshift |
| 3878delG | deletion of G at 3878 | 20 | frameshift mutation at 1249 |
| | | | and stop codon at 1258 |
| 3891 G/A | G or A at 3891 | 20 | Sequence Variation |
| 3898insC | insertion of C after 3898 | 20 | frameshift |
| 3905insT | insertion of T after 3905 | 20 | frameshift |
| 3906insG | insertion of G after 3906 | 20 | frameshift |
| 3922del10->C | deletion of 10 bp from 3922 and | 20 | deletion of Glu1264 to |
| | replacement with 3921 | | Glu1266 |
| 3939C/T | C or T at 3939 | 20 | sequence variation |
| 3944delGT | deletion of GT from 3944 | 20 | frameshift |
| 394delTT | deletion of TT from 394 | 3 | frameshift |
| 3960 − 3961delA | Deletion of A at 3960 − 3961 | 20 | Frameshift |
| 4005 + 117T/G | T or G at 4005 + 117 | intron 20 | sequence variation |
| 4005 + 121delTT | 8T or 6T at 4005 + 121 | intron 20 | sequence variation |
| 4005 + 1G->A | G to A at 4005 + 1 | intron 20 | mRNA splicing defect |
| 4005 + 23delA | Deletion of A at 4005 + 23 | Intron 20 | Sequence variation - mRNA |
| | | | splicing defect |
| 4005 + 28insA | 6A or 7A at 4005 + 28 | intron 20 | sequence variation |
| 4005 + 29G->C | G to C at 4005 + 29 | intron 20 | Splicing |
| 4005 + 2T->C | T to C at 4005 + 2 | intron 20 | mRNA splicing defect |
| 4005 + 33A->G | A to G at 4005 + 33 | intron 20 | Splicing |
| 4006 − 103delT | deletion of T at 4006 − 103 | intron 20 | sequence variation |
| 4006 − 11 t->G | T to G ar 4006 − 11 | | mRNA splicing defect |
| 4006 − 14C->G | C to G at 4006 − 14 | intron 20 | mRNA splicing defect |
| 4006 − 19del3 | deletion of 3 bp from 4006 − 19 | intron 20 | mRNA splicing defect |
| 4006 − 200G/A | G or A at 4006 − 200 | intron 20 | sequence variation |
| 4006 − 26 T/C | 4006 − 26 T > C | intron 20 | sequence variation |
| 4006 − 46delTATTT | Deletion from 4006 − 46 to 4006 − | intron 20 | Splicing defect |
| | 42 | | |
| 4006 − 4A->G | A to G at 4006 − 4 | intron 20 | mRNA splicing defect |
| 4006 − 50 A/C | 4006 − 50 A > C | intron 20 | sequence variation |
| 4006 − 61del14 | deletion of 14 bp from 4006 − 61 to | intron 20 | mRNA splicing defect |
| | 4006 − 47 | | |
| 4006 − 8T->A | T to A at 4006 − 8 | intron 20 | mRNA splicing defect |
| 4006delA | deletion of A at 4006 | 21 | frameshift |
| 4010del4 | deletion of TATT from 4010 | 21 | frameshift |
| 4015delA | deletion of A at 4015 | 21 | frameshift |
| 4016insT | insertion of T at 4016 | 21 | frameshift |
| 4022insT | insertion of T at 4022 | 21 | Frameshift. |
| 4029A/G | A or G at 4029 | 21 | sequence variation |
| 4040delA | deletion of A at 4040 | 21 | frameshift |
| 4041_4046del6insTGT | Deletion of nucleotides 4041 to | 21 | deletion of Leu at 1304 and |
| | 4046 and insertion of TGT | | Asp at 1305, insertion of Val at |
| | | | 1304 |
| 4048insCC | insertion of CC after 4048 | 21 | frameshift |
| 405 + 1G->A | G to A at 405 + 1 | intron 3 | mRNA splicing defect |
| 405 + 3A->C | A to C at 405 + 3 | intron 3 | mRNA splicing defect |
| 405 + 42A/G | A or G at 405 + 42 | intron 3 | sequence variation |
| 405 + 46G/T | G or T at 405 + 46 | intron 3 | sequence variation |
| 405 + 4A->G | A to G at 405 + 4 | intron 3 | mRNA splicing defect |
| 406 − 10C->G | C to G at 406 − 10 | intron 3 | mRNA splicing defect |
| 406 − 112T/A | T or A at 406 − 112 | intron 3 | sequence variation |
| 406 − 13T/C | T or C at 406 − 13 | intron 3 | sequence variation |
| 406 − 1G->A | G to A at 406 − 1 | intron 3 | mRNA splicing defect |
| 406 − 1G->C | G to C at 406 − 1 | intron 3 | mRNA splicing defect |
| 406 − 1G->T | G to T at 406 − 1 | intron 3 | mRNA splicing defect |
| 406 − 2A->C | A to C at 406 − 2 | intron 3 | mRNA splicing defect |
| 406 − 2A->G | A to G at 406 − 2 | intron 3 | mRNA splicing defect |
| 406 − 3T->C | T to C at 406 − 3 | intron 3 | mRNA splicing defect |
| 406 − 5T->G | T to G at 406 − 5 | intron 3 | mRNA splicing defect |
| 406 − 6T->C | T to C at 406 − 6 | intron 3 | mRNA splicing defect |
| 406 − 82T/A | T or A at 406 − 82 | Intron 3 | Sequence variation |
| 406 − 83A/G | A or G at 406 − 83 | intron 3 | sequence variation |
| 4086T/C | T or C at 4086 | 21 | sequence variation |
| 4095 + 1G > C | 4095 + 1 G > C | intron 21 | mRNA splicing defect |
| 4095 + 1G->T | 4095 + 1G > T | Intron 21 | mRNA splicing defect |
| 4095 + 2T->A | 4095 + 2 T > A | intron 21 | mRNA slicing defect |
| 4095 + 42T/C | T or C at 4095 + 42 | intron 21 | sequence variation |
| 4096 − 1G->A | G to A at 4096 − 1 | intron 21 | mRNA splicing defect |
| 4096 − 283T/C | T or C at 4096 − 283 | intron 21 | sequence variation |
| 4096 − 28G->A | G to A at 4096 − 28 | intron 21 | mRNA splicing defect |
| 4096 − 3C->G | C to G at 4096 − 3 | intron 21 | mRNA splicing defect |
| 40G/C | G to C at 40 | 1 | Sequence variation |
| 4108delT | deletion of T at 4108 | 22 | frameshift |
| 4114ATA->TT | ATA to TT from 4114 | 22 | Ile to Leu at 1328 and |
| | | | frameshift |
| 412del7->TA | deletion of ACCAAAG from 412 | 4 | frameshift |
| | and insertion of TA | | |
| 4168delCTAAGCC | Deletion of CTAAGCC at 4168 | 22 | |
| 4171insA | insertion of A at 4171 | 22 | Frameshift a premature stop |
| | | | codon appears 12 codons |
| | | | further. |
| 4172delGC | deletion of GC from 4172 | 22 | frameshift |
| 4173delC | deletion of C at 4173 | 22 | frameshift |
| 4203TAG->AA | TAG to AA at 4203 | 22 | frameshift |
| 4209TGTT->AA | TGTT to AA from 4209 | 22 | Frame shift |
| 4218insT | insertion of T after 4218 | 22 | frameshift |
| 4269 − 108A->G | A to G at 4269 − 108 | intron 22 | sequence variation |
| 4269 − 139G/A | G or A at 4269 − 139 | intron 22 | sequence variation |
| 4271delC | deletion of C at 4271 | 23 | frameshift |
| 4272delA | Deletion of nucleotide A at 4272 | 23 | Frameshift |
| | position | | |
| 4279insA | insertion of A after 4279 | 23 | frameshift |
| 4301)delA | deletion of A at 4301 or 4302 | 23 | frameshift |
| 4326delTC | Deletion of TC from 4326 to 4327 | 23 | FrameShift |
| 4326delTC | deletion of TC from 4326 | 23 | frameshift |
| 4329C/G | C or G at 4329 | Exon 23 | Sequence Variation |
| 4332delTG | deletion of TG at 4332 | 23 | framshift |
| 4356G/A | G or A at 4356 | 23 | sequence variation |
| 435insA | insertion of A after 435 | 4 | frameshift |
| 4374 + 10T->C | T to C at 4374 + 10 | intron 23 | splicing |
| 4374 + 13A/G | A or G at 4374 + 13 | intron 23 | sequence variation |
| 4374 + 14A/G | A or G at 4374 + 14 | intron 23 | sequence variation |
| 4374 + 1G->A | G to A at 4374 + 1 | intron 23 | mRNA splicing defect |
| 4374 + 1G->T | G to T at 4374 + 1 | intron 23 | mRNA splicing defect |
| 4374_4374 + 1GG>TT | 4374_4374 + 1GG>TT | 23, | mRNA splicing defect |
| | | intron23 | |
| 4375 − 15C/T | C or T at 4375 − 15 | intron 23 | sequence variation |
| 4375 − 1G->C | G to C at 4375 − 1 | intron 23 | splicing mutation |
| 4375 − 36delT | deletion of T at 375 − 36 | intron 23 | sequence variation |
| 4382delA | deletion of A at 4382 | 24 | frameshift |
| 4404C/T | C or T at 4404 | 24 | sequence variation |
| 441delA | deletion of A at 441 and T to A at | 4 | frameshift |
| | 486 | | |
| 4428insGA | insertion of GA after 4428 | 24 | frameshift |
| 444delA | deletion of A at 444 | 4 | frameshift |
| 4464 C/T | C to T at 4464 | 24 | sequence variation |
| 451del8 | deletion of GCTTCCTA from 451 | 4 | frameshift |
| 4521G/A | G or A at 4521 | 24 | sequence variation |
| 4557 G/A | G to A at 4557 | 24 | sequence variation (Leu at |
| | | | 1475 no change) |
| 4563T/C | T or C at 4563 | 24 | sequence variation |
| 4575 + 2G->A | G to A at 4575 + 2 | intron 24 | Splicing |
| 457TAT->G | TAT to G at 457 | 4 | frameshift |
| 458delAT | deletion of AT at 458 | 4 | frameshift |
| 4608 − 4638del31 | 31bp deletion between 4608 and | intron 24 | sequence variation |
| | 4638 | | |
| 460delG | deletion of G at 460 | 4 | frameshift |
| −461A->G | A to G at −461 | promotor | Sequence variation |
| 4655T->G | T to G at 4655 | intron 24 | sequence variation |
| 465G/A | G or A at 465 | 4 | sequence variation |
| 4700T8/9 | 8T or 9T at 4700 | intron 24 | sequence variation |
| −471delAGG | deletion of AGG from −471 | promotor | promoter mutation |
| 489 C/T | C to T at 489 | 4 | sequence variation |
| 489delC | deletion of C at 489 | 4 | frameshift |
| 48C/G | C or G at 48 | promotor | sequence variation |
| 492G/A | G or A at 492 | 4 | sequence variation |
| 519delT | T deleted | 4 | frameshift |
| 525delT | deletion of T at 525 | 4 | frameshift |
| 541del4 | deletion of CTCC from 541 | 4 | frameshift |
| 541delC | deletion of C at 541 | 4 | frameshift |
| 545T/C | T or C at 545 | 4 | sequence variation |
| 546insCTA | insertion of CTA at 546 | 4 | frameshift |
| 547insGA | insertion of GA between | 4 | Frameshift; a premature stop |
| | nucleotides 547 and 548 | | codon appears 15 codons |
| | | | further. |
| 547insTA | insertion of TA after 547 | 4 | frameshift |
| 549C/T | C to T at 549 | 4 | sequence variation (His at 139 |
| | | | no change) |
| 552insA | insertion of A after 552 | 4 | frameshift |
| 556delA | deletion of A at 556 | 4 | frameshift |
| 557delT | deletion of T at 557 | 4 | frameshift |
| 565delC | deletion of C at 565 | 4 | frameshift |
| 574delA | deletion of A at 574 | 4 | frameshift |
| 576InsCTA | Insert CTA at 576 | 4 | In frame in/del |
| −589G/A | G or A at −589 | Promoter | Sequence Variation |
| 591del18 | deletion of 18 bp from 591 | 4 | deletion of 6 a.a. from |
| 605insT | insertion of T after 605 | 4 | frameshift |
| 612T/A | T or A at 612 (together with | 4 | sequence variation |
| | Y161S) | | |
| 621 + 1G->T | G to T at 621 + 1 | intron 4 | mRNA splicing defect |
| 621 + 2T->C | T to C at 621 + 2 | intron 4 | mRNA splicing defect |
| 621 + 2T->G | T to G at 621 + 2 | intron 4 | mRNA splicing defect |
| 621 + 31C/G | C or G at 621 + 31 | intron 4 | sequence variation |
| 621 + 3A->G | A to G at 621 + 3 | intron 4 | mRNA splicing defect |
| 621G->A | G to A at 621 | 4 | mRNA splicing defect |
| 622 − 103A/G | A or G at 622 − 103 | intron 4 | sequence variation |
| 622 − 116A/G | A or G at 622 − 116 | intron 4 | sequence variation |
| 622 − 152G/C | G or C at 622 − 152 | intron 4 | sequence variation |
| 622 − 16 T/C | 622 − 16 T > C | intron 4 | sequence variation |
| 622 − 1G->A | G to A at 622 − 1 | intron 4 | mRNA splicing defect |
| 622 − 2A->C | A to C at 622 − 2 | intron 4 | mRNA splicing defect |
| 622 − 2A->G | A to G at 622 − 2 | intron 4 | mRNA splicing defect |
| 624delT | deletion of T at 624 | 5 | frameshift |
| 650delATAAA | Deletion of ATAAA at 650 | 5 | Frameshift |
| 657delA | deletion of A at 657 | 5 | frameshift |
| 663delT | deletion of T at 663 | 5 | frameshift |
| 675del4 | deletion of TAGT from 675 | 5 | frameshift |
| 676A/G | A or G at 676 | 5 | sequence variation |
| 681delC | deletion of C at 681 | 5 | frameshift |
| 710_711 + 5del7 | Deletion of AAGTATG between | 5 | |
| | 710 and 711 + 5 | | |
| 711 + 1G->T | G to T at 711 + 1 | intron 5 | mRNA splicing defect |
| 711 + 34A->G | A to G at 711 + 34 | intron 5 | mRNA splicing defect |
| 711 + 3A->C | A to C at 711 + 3 | intron 5 | mRNA splicing defect |
| 711 + 3A->G | A to G at 711 + 3 | intron 5 | mRNA splicing defect |
| 711 + 3A->T | A to T at 711 + 3 | intron 5 | mRNA splicing defect |
| 711 + 5G->A | G to A at 711 + 5 | intron 5 | mRNA splicing defect |
| 712 − 1G->T | G to T at 712 − 1 | intron 5 | mRNA splicing defect |
| 712 − 92T/A | T or A at 712 − 92 | intron 5 | sequence variation |
| 733delG | Deletion of G at 733 | 6a | Frameshift |
| 741C/T | C or T at 741 | 6a | sequence variation |
| −741T->G | T to G at −741 | promotor | promoter mutation |
| 759A/G (A209A)) | A or G at 759 | 6a | sequence variation (Ala at 209 |
| | | | no change) |
| −790T9/8 | 9T or 8T at −790 | promotor | sequence variation |
| 795G/A | G to A at 795 | 6a | Sequence variation. No |
| | | | change |
| −816C->T | C to T at −816 | promotor | promoter mutation |
| −816delCTC | deletion of CTC at −816 | promotor | sequence variation |
| −834T/G | T or G at −834 | promotor | sequence variation |
| 852del22 | deletion of 22 bp from 852 | 6a | frameshift |
| 873C/T | C or T at 873 | 6a | sequence variation |
| 874Ins TACA | Insertion of 4 bp (TACA) at 874 | 6a | stop codon at amino acid 257 |
| | | | in exon 6b |
| 875 + 1G->A | G to A at 875 + 1 | intron 6a | mRNA splicing defect |
| 875 + 1G->C | G to C at 875 + 1 | intron 6a | mRNA splicing defect |
| 875 + 40A/G | A or G at 875 + 40 | intron 6a | sequence variation |
| 876 − 10del8 | deletion of 8 bp from 876 − 10 | intron 6a | mRNA splicing defect |
| 876 − 14del12 | deletion of 12 bp from 876 − 14 | intron 6a | mRNA splicing defect |
| 876 − 3C->T | C to T at 876 − 3 | intron 6a | splicing mutation |
| 876 − 8A->C | 876 − 8A > C | intron 6a | mRNA splicing defect |
| −895T/G | T or G at −895 upstream of the | promotor | sequence variation |
| | cap site | | |
| 905delG | deletion of G at 905 | 6b | frameshift |
| −912dupT | deletion of T at nucleotide −912 | promotor | Sequence variation |
| 935delA | deletion of A at 935 | 6b | frameshift |
| 936delTA | deletion of TA from 936 | 6b | frameshift |
| −94G->T | G to T at −94 | promotor | promoter mutation |
| 977insA | insertion of A after 977 | 6b | frameshift |
| 989 − 992insA | Insertion of A at 989 − 992 | 6b | Frameshift |
| 991del5 | deletion AACTT from 991 or | 6b | frameshift |
| | CTTAA from 993 | | |
| 994del9 | deletion of TTAAGACAG from 994 | 6b | mRNA splicing defect |
| 99C/T | C or T at 99 | promotor | sequence variation |
| A1006E | C to A at 3149 | 17a | Ala to Glu at 1006 |
| A1009T | G to A at 3157 | 17a | Ala to Thr at 1009 |
| A1025D | C to A at 3206 | 17a | Substitution of alanine to |
| | | | aspartic acid at position 1025 |
| A1067D | C to A at 3332 | 17b | Ala to Asp at 1067 |
| A1067G | C to G at 3332 | 17b | Ala to Gly at 1067 |
| A1067P | G to C at 3331 | 17b | Ala en Pro at 1067 |
| A1067T | G to A at 3331 | 17b | Ala to Thr at 1067 |
| A1067V | C to T at 3332 | 17b | Ala to Val at 1067 |
| A107G | C to G at 452 | 4 | Ala to Gly at 107 |
| A1081P | G to C at 3373 | 17b | Ala to Pro at 1081 |
| A1087P | G to C at 3391 | 17b | Ala to Pro at AS 1087 |
| A1136T | G to A at 3538 | 18 | Ala to Thr at 1136 |
| A120T | G to A at 490 | 4 | Ala to Thr at 120 |
| A120V | C to T at 491 | 4 | Ala to Val at 120 |
| A1319E | C to A at 4088 | 21 | Ala to Glu at 1319 |
| A1364V | C to T at 4223 | 22 | Ala to Val at 1364 CBAVD |
| A141D | C to A at 554 | 4 | Ala to Asp at 141 |
| A155P | G to C at 595 | 4 | Ala to Pro at 155 |
| A198P | G to C at 724 | 6a | Ala to Pro at 198 |
| A209S | G to T at 757 | 6a | Ala to Ser at 209 |
| A238V | C to T at 845 | 6a | Ala to Val at 238 |
| A299T | G to A at 1027 | 7 | Ala to Thr at 299 |
| A309A (1059C/G) | C or G at 1059 | 7 | sequence variation |
| A309D | C to A at 1058 | 7 | Ala to Asp at 309 |
| A309G | C to G at 1058 | 7 | Ala to Gly at 309 |
| A309T | G to A at 1057 | 7 | Ala to Thr at 309 |
| A309V | C to T at 1058 | 7 | Ala to Val at 309 |
| A349V | C to T at 1178 | 7 | Ala to Val at 349 |
| A399D | C to A at 1328 | 8 | Ala to Asp at 399 |
| A399V | C to T at 1328 | 8 | Ala to Val at 399 |
| A455E | C to A at 1496 | 9 | Ala to Glu at 455 |
| A46D | C to A at 269 | 2 | Ala to Asp at 46 |
| A534E | C to A at 1733 | 11 | Ala to Glu at 534 |
| A559E | C to A at 1808 | 11 | Ala to Glu at 559 |
| A559T | G to A at 1807 | 11 | Ala to Thr at 559 |
| A559V | C to T at 1808 | 11 | Ala to Val at 559 |
| A561E | C to A at 1814 | 12 | Ala to Glu at 561 |
| A566T | G to A at 1828 | 12 | Ala to Thr at 566 |
| A613T | G to A at 1969 | 13 | Ala to Thr at 613 |
| A72D | C to A at 347 | 3 | Ala to Asp at 72 |
| A72T | G to A at 346 | 3 | Ala to Thr at 72 |
| A800G | C to G at 2531 | 13 | Ala to Gly at 800 |
| A959V | C to T at 3008 | 15 | Ala to Val at 959 |
| A96E | C to A at 419 | 4 | Ala to Glu at 96 |
| C225R | T to C at 805 | 6a | Cys to Arg at 225 |
| C225X | T to A at 807 | 6a | Cys to Stop at 225 |
| C276X | C to A at 960 | 6b | Cys to Stop at 276 |
| C491R | T to C at 1603 | 10 | Cys to Arg at 491 |
| C524X | C to A at 1704 | 10 | Cys to Stop at 524 |
| C866R | T to G at 2728 | 14a | Cys to Arg at 866 |
| C866S | T to A at 2728 | 14a | Cys to Ser at 866 |
| C866Y | G to A at 2729 | 14a | Cys to Tyr at 866 |
| CF25kbdel | Complex deletion/rearrangement | intron 3 | |
| CFTR40kbdel | deletion of exons 4-10 | 4, 5, 6a, | large deletion from intron 3 to |
| | | 6b, 7, 8, | intron 10 |
| | | 9, 10 | |
| CFTR40kbdel | deletion of exons 4-10 | 4, 5, 6a, | large deletion from intron 3 to |
| | | 6b, 7, 8, | intron 10 |
| | | 9, 10 | |
| CFTR50kbdel | complex deletion involving exons | 4, 5, 6a, | complex deletion |
| | 4-7 and 11-18 | 6b, 7, 11, | |
| | | 12, 13, | |
| | | 14a, 14b, | |
| | | 15, 16, | |
| | | 17a, 17b, | |
| | | 18 | |
| CFTRdele1 | Deletion of exon 1 from nucleotide | 1 | A small peptide of 17 residues |
| | 136 (codons 2-18) to intron 1 | | if translation starts at the same |
| | nucleotide + 69 and insertion of an | | ATG or another protein |
| | inverted and complementary | | (possibly CFTR-like) if another |
| | sequence of intron 1 (nucleotide | | ATG is choosen. |
| | 185 + 4191 to + 4488) and addition | | |
| | of a G at the junction. | | |
| CFTRdele11-16Ins35bp | Gross deletion of 47.5 kb going | 11, 12, | The in-frame deletion of exons |
| | from IVS10 + 12 to IVS16 + 403 | 13, 14a, | 11 to 16 was predicted to |
| | that removed exons 11 to 16 | 14b, 15, | result in a protein lacking |
| | inclusive, together with an | 16 | amino acids 529 to 996; this |
| | insertion of 35 bp | | includes the carboxy terminal |
| | | | end of NBD1, the entire |
| | | | regulatory R domain and |
| | | | transmembrane-spanning |
| | | | regions TM7 and TM8. |
| CFTRdele1-24 | deletion of the whole CFTR gene | 1, 2, 3, 4, | absence of CFTR expression. |
| | | 5, 6a, 6b, | |
| | | 7, 8, 9, | |
| | | 10, 11, | |
| | | 12, 13, | |
| | | 14a, 14b, | |
| | | 15, 16, | |
| | | 17a, 17b, | |
| | | 18, 19, | |
| | | 20, 21, | |
| | | 22, 23, | |
| | | 24 | |
| CFTRdele14a | deletion of >=1.2 kb including | 14a | aberrant mRNA splicing |
| | exon 14a | | |
| CFTRdele14b-17b | 9890 bp deletion | 14b, 15, | Removes 5 coding exons |
| | | 16, 17a, | |
| | | 17b | |
| CFTRdele14b-18 | deletion of 20 kb from exons 14b | 14b, 15, | deletion of amino acids 874-1156 |
| | through 18 | 16, 17a, | |
| | | 17b, 18 | |
| CFTR-dele 16-17a-17b | 3040 + 1085_3499 + 260del7201 | 16, 17a, | Large in frame deletion |
| | | 17b | removing exons 16, 17a, 17b |
| CFTRdele16-17b | deletion of 7kb starting at intron 15 | 16, 17a, | large deletion from intron 15 to |
| | | 17b | intron 17b |
| CFTRdele17b18 | deletion of exons 17b and 18 | 17b, 18 | frameshift |
| CFTRdele19 | deletion of 5.3kb, removing exon | 19 | |
| | 19 | | |
| CFTRdele1Ins299bp | This indel involved the deletion of | 1 | |
| | 119 bp extending from coding | | |
| | position 4 (A of the ATG- | | |
| | translation initiation codon being | | |
| | defined as 1) to IVS1 + 69 that | | |
| | removed nearly the entire coding | | |
| | sequence of exon 1, and the | | |
| | insertion of 299 bp at the deletion | | |
| | junction | | |
| CFTRdele1 or | 136_185 + 69del119bpins299bp | 1, intron 1 | Deletion of exon 1 from |
| 136del119ins299 | | | nucleotide 136 (codons 2-18) |
| | | | to intron 1 nucleotide + 69 and |
| | | | insertion of an inverted and |
| | | | complementary sequence of |
| | | | intron 1 (nucleotide 185 + 4191 |
| | | | to + 4488) and addition of a G |
| | | | at the junction. A small peptide |
| | | | of 17 residues if translation |
| | | | starts at the same ATG or |
| | | | another protein (possibly |
| | | | CFTR-like) if another ATG is |
| | | | choosen. |
| CFTRdele2 | deletion of exon 2 | 2 | frameshift |
| CFTR-dele2 | 186-1161_296 + 1603del2875 | 2 | Large in frame deletion |
| | | | removing exon2 |
| CFTRdele21 | deletion of exon 21 | 21 | large deletion from exon 21 |
| CFTRdele2-10 | deletion of 95.7 kb starting in | 2, 3, 4, 5, | frameshift |
| | intron 1 | 6a, 6b, 7, | |
| | | 8, 9, 10 | |
| CFTRdele22, 23 | This deletion extends from | 22, 23 | The loss of exons 22 and 23 |
| | nucleotide − 78 of intron 21 (the | | was in-frame and was |
| | end of intron 21 being defined as − | | predicted to result in a CFTR |
| | 1) to nucleotide + 577 of intron 23 | | protein lacking amino acids |
| | (the beginning of intron 23 being | | 1322 to 1414; this constitutes |
| | defined as + 1) with a loss of 1532 | | the carboxy terminal end of |
| | nucelotides | | the newly defined nucleotide- |
| | | | binding domain (NBD) 2 of the |
| | | | protein |
| CFTRdele2, 3 | deletion of exons 2 and 3 | 2, 3 | frameshift |
| CFTRdele3-10, 14b-16 | Complex deletion involving exons | 3, 4, 5, | Complex deletion |
| | 3-10 and 14b-16 | 6a, 6b, 7, | |
| | | 8, 9, 10, | |
| | | 14b, 15, | |
| | | 16 | |
| CFTRdele3-10, 14b-16 | Complex deletion involving exons | 3, 4, 5, | Complex deletion |
| | 3-10 and 14b-16 | 6a, 6b, 7, | |
| | | 8, 9, 10, | |
| | | 14b, 15, | |
| | | 16 | |
| CFTRdele4-6aIns6bp | Deletion of 18, 654 bp | 4, 5, 6a | This large deletion disrupted |
| | encompassing exons 4, 5, and 6a, | | the reading frame of the |
| | together with an insertion of 6 bp | | protein |
| CFTRdele4Ins41bp | Gross deletion of 8, 165 bp | 4 | This deletion was in-frame and |
| | spanning exon 4, together with an | | was predicted to lead to the |
| | insertion of 41 bp | | synthesis of a protein lacking |
| | | | amino acids 92-163, a stretch |
| | | | that includes a part of TM1 |
| | | | and the the entire TM2 |
| CFTRdup10_18 | Duplication of exons 10 to 18 | 10, 11 | The position and orientation of |
| | | 12, 13, | the duplicated region have not |
| | | 14a, 14b, | been determined. However, |
| | | 15, 16, | given the classical CF |
| | | 17a, 17b, | phenotype, it is hypothesized |
| | | 18 | that it is located inside the |
| | | | CFTR gene. |
| CFTRdup11_13 | Duplication of exons 11 to 13 | 11, 12 | The position and orientation of |
| | | 13 | the duplicated region have not |
| | | | been determined. |
| CFTRdup1-3 | Duplication of exons 1 to 3 | 1, 2, 3 | Large rearrangement. The |
| | | | break points and orientation |
| | | | are being assessed |
| CFTRdup4-8 | Duplication of exons 4 to 8 | 4, 5, 6, 7, 8 | Complex rearrangement. The |
| | | | position and orientation of the |
| | | | duplicated region is not |
| | | | determined so far. However, |
| | | | given the classical CF |
| | | | phenotype, it is hypothesized |
| | | | that it is located inside the |
| | | | CFTR gene. |
| Complex repeats | | intron | sequence variation |
| | | 17b | |
| D110E | C to A at 462 | 4 | Asp to Glu at 110 |
| D110H | G to C at 460 | 4 | Asp to His at 110 |
| D110N | G to A at 460 | 4 | Asp to Asn at 110 |
| D110Y | G to T at 460 | 4 | Asp to Tyr at 110 |
| D1152H | G to C at 3586 | 18 | Asp to His at 1152 |
| D1154G | A to G at 3593 | 18 | Asp to Gly at 1154 (CBAVD) |
| D1154Y | G to T at 3592 | 18 | Asp to Tyr at 1154 |
| D1168G | A to G at 3635 | 19 | Asp to Gly at 1168 |
| D1270N | G to A at 3940 | 20 | Asp to Asn at 1270 |
| D1270Y | G to T at 3940 | 20 | Asp to Tyr at 1270 |
| D1305E | T to A at 4047 | 21 | Asp to Glu at 1305 |
| D1312G | A to G at 4067 | 21 | Asp to Gly at 1312 |
| D1377H | G to C at 4261 | 22 | Asp to His at 1377 |
| D1445N | G to A at 4465 | 24 | Asp to Asn at 1445 |
| D192G | A to G at 707 | 5 | Asp to Gly at 192 |
| D192N | G to A at 706 | 5 | Asp to Asn at 192 |
| D36N | G to A at 238 | 2 | Asp to Asn at 36 |
| D373E | T to G at 1251 | 8 | Asp to Glu a 373 |
| D443Y | G to T at 1459 | 9 | Asp to Tyr at 443 |
| D44G | A to G at 263 | 2 | Asp to Gly at 44 |
| D513G | A to G at 1670 | 10 | Asp to Gly at 513 (CBAVD) |
| D529G | A to G at 1718 | 11 | Asp to Gly at 529 |
| D529H | G to C at 1717 | 11 | Asp to His at 529 |
| D537E | C to A or C to G at 1743 | 11 | Asp to Glu at 537 |
| D565G | A to G at 1826 | 12 | Asp to Gly at 565 |
| D572N | G to A at 1846 | 12 | Asp to Asn at 572 |
| D579A | A to C at 1868 | 12 | Asp to Ala at 579 |
| D579G | A to G at 1868 | 12 | Asp to Gly at 579 |
| D579Y | G to T at 1867 | 12 | Asp to Tyr at 579 |
| D58G | A to G at 305 | 3 | Asp to Gly at 58 |
| D58N | G to A at 304 | 3 | Asp to Asn at 58 |
| D614G | A to G at 1973 | 13 | Asp to Gly at 614 |
| D614Y | G to T at 1972 | 13 | Asp to Tyr at 614 |
| D639Y | G to T at 2047 | 13 | Asp to Tyr at 639 |
| D651H | G to C at 2083 | 13 | Asp to His at 651 |
| D651N | G to A at 2083 | 13 | Asp to Asn at 651 |
| D674V | A to T at 2153 | 13 | Asp to Val at 674 |
| D806G | A to G at 2549 | 13 | Asp to Gly at 806 |
| D828G | A to G at 2615 | 13 | Asp to Gly at 828 |
| D836Y | G to T at 2638 | 14a | Asp to Tyr at 836 |
| D891G | A to G at 2804 | 15 | Asp to Gly at 891 |
| D924N | G to A at 2902 | 15 | Asp to Asn at 924 |
| D979A | A to C at 3068 | 16 | Asp to Ala at 979 (CBAVD) |
| D979V | A to T at 3068 | 16 | Asp to Val at 979 |
| D985H | G to C at 3085 | 16 | Asp to His at 985 |
| D985Y | G to T at 3085 | 16 | Asp to Tyr at 985 |
| D993G | A to G at 3110 | 16 | Asp to Gly at 993 |
| D993Y | G to T at 3109 | 16 | Asp to Tyr at 993 |
| delePr-3 | Large deletion | promotor, | |
| | | 1, 2, 3 | |
| delEx2-6b | 185 + 2909_1002 − | 2, intron | The deletion of exons 2 to 6b |
| | 1620del155429ins17 ((insertion of | 2, 3, | is in frame and would lead to |
| | GTACTCAACAGCTCTAG (SEQ | intron 3, | remove 272 residues. |
| | ID NO: 11)) | 4, intron | |
| | | 4, 5, | |
| | | intron 5, | |
| | | 6a, intron | |
| | | 6a, 6b, | |
| | | intron 6b | |
| delEx2-9 | c.53 + 9?711_1392 + | 1, intron | Large deletion of exons 2-9 |
| | 2?670del61?634 | 1, 2, | (intron 1 to intron 9), out of |
| | | intron 2, | frame |
| | | 3, intron | |
| | | 3, 4, | |
| | | intron 4, | |
| | | 5, intron | |
| | | 5, 6a, | |
| | | intron 6a, | |
| | | 6b, intron | |
| | | 6b, 7, | |
| | | intron 7, | |
| | | 8, intron | |
| | | 8, 9, | |
| | | intron 9 | |
| Del exon 17a-17b | Deletion of exons 17a-17b | 17a, 17b | Truncation of CFTR protein in |
| | | | TM2. |
| Del exon 17a-17b-18 | Deletion of exons 17a-18 | 17a, 17b, | in-frame deletion, joining of |
| | | 18 | exons 16 to 19; deletion of |
| | | | terminal domain of TM2. |
| Del exon 22-23 | Deletion of exons 22-23 | 22, 23 | In-frame deletion that is |
| | | | predicted to remove the |
| | | | terminal part of NBD2 |
| Del exon 22-24 | Deletion of Exons 22, 23, 24 | 22, 23, | Predicted Removal of terminal |
| | | 24 | portion of CFTR protein |
| Del exon 2-3 | Deletion of exons 2, 3 | 2, 3 | Predicted truncation of the |
| | | | CFTR Protein |
| Del exon 4-6a | Deletion of exons 4, 5, 6a | 4, 5, 6a | Predicted truncation of the |
| | | | CFTR protein in TM1. |
| Del Pr-Ex1 | Deletion of Promoter, Exon 1 | promotor, 1 | Predicted Removal of CFTR |
| | | | gene expression and ATG |
| | | | start Codon. |
| Del Pr-Ex1-Ex2 | Deletion of Promoter, Exon 1, | promotor, | Predicted Removal of CFTR |
| | Exon 2 | 1, 2 | gene expression and ATG |
| | | | start Codon. |
| [delta]D192 | deletion of TGA or GAT from 706 | 5 | deletion of Asp at 192 |
| | or 707 | | |
| [delta]E115 | 3 bp deletion of 475-477 | 4 | deletion of Glu at 115 |
| [delta]F311 | deletion of 3 bp between 1059 and | 7 | deletion of Phe310, 311 or |
| | 1069 | | 312 |
| [delta]F508 | deletion of 3 bp between 1652 and | 10 | deletion of Phe at 508 |
| | 1655 | | |
| [delta]I507 | deletion of 3 bp between 1648 and | 10 | deletion of Ile506 or Ile507 |
| | 1653 | | |
| [delta]L1260 | deletion of ACT from either 3909 | 20 | deletion of Leu at 1260 or |
| | or 3912 | | 1261 |
| [delta]L453 | deletion of 3 bp between 1488 and | 9 | deletion of Leu at 452 or 454 |
| | 1494 | | |
| [delta]M1140 | deletion of 3 bp between 3550 and | 18 | deletion of Met at 1140 |
| | 3553 | | |
| [delta]T339 | deletion of 3 bp between 1148 and | 7 | deletion of Thr at 1140 |
| | 1150 | | |
| dup1716 + 51->61 | duplication of 11 bp at 1716 + 51 | intron 10 | sequence variation |
| Dup ex 6b-10 (gIVS6a + | A duplication of exons 6b-10. The | 6b, 7, 8, | Out-of-frame fusion of exon 10 |
| 415_IVS10 + | duplication is 26817 bp long. | 9, 10 | to exon 6b |
| 2987Dup26817bp) | | | |
| E1104X | G to Tat 3442 | 17b | Glu to Stop at 1104 |
| E1123del | Deletion of AAG at 3503-3505 | 18 | deletion of Glu at 1123 |
| E116K | G to A at 478 | 4 | Glu to Lys at 116 |
| E116Q | G to C at 478 | 4 | Glu to Gln at 116 |
| E1228G | A to G at 3815 | 19 | Glu to Gly at 1228 |
| E1308X | G to T at 4054 | 21 | Glu to Stop at 1308 |
| E1321Q | G to C at 4093 | 21 | Glu to Gln at 1321 |
| E1371X | G to T at 4243 | 22 | Glu to Stop at 1371 |
| E1401G | A to G at 4334 | 23 | Glu to Gly at 1401 |
| E1401K | G to A at 4333 | 23 | Glu to Lys at 1401 |
| E1401X | G to T at 4333 | 23 | Glu to Stop at 1401 |
| E1409K | G to A at 4357 | 23 | Glu to Lys at 1409 |
| E1409V | A to T at 4358 | 23 | Glu to Val at 1409 |
| E1418X | G to T at 4384 (GAG->TAG) | 24 | Glu to Stop at 1418 |
| E1473X | G to T at 4549 | 24 | Glu to Stop at 1473 |
| E193K | G to A at 709 | 5 | Glu to Lys at 193 |
| E193X | G to T at 709 | 5 | Glu to Stop at 193 |
| E217G | A to G at 782 | 6a | Glu to Gly at 217 |
| E278del | deletion of AAG from 965 | 6b | deletion of Glu at 278 |
| E279D | A to T at 969 | 6b | Glu to Asp at 279 |
| E279D | A to T at 969 | 6b | Glu to Asp at 279 |
| E292K | G to A at 1006 | 7 | Glu to Lys at 292 |
| E379K | G to A at 1267 | 8 | Glu to Lys at 379 |
| E379X | G to T at 1267 | 8 | Glu to Stop at 379 |
| E403D | G to C at 1341 | 8 | Glu to Asp at 403 |
| E407V | A to T at 1352 | 9 | Glu to Val at 407 |
| E474K | G to A at 1552 | 10 | Glu to Lys at 474 |
| E479X | G to T at 1567 | 10 | Glu to Stop at 479 |
| E504Q | G to C at 1642 | 10 | Glu to Gln at 504 |
| E504X | G to T at 1642 | 10 | Glu to Stop at 504 |
| E527G | A to G at 1712 | 10 | Glu to Gly at 527 |
| E527Q | G to C at 1711 | 10 | Glu to Gln at 527 |
| E528D | G to T at 1716 | 10 | Glu to Asp at 528 (splice |
| | | | mutation) |
| E528K | G to A at 1714 | 10 | Glu to Lys at 528 |
| E56K | G to A at 298 | 3 | Glu to Lys at 56 |
| E585X | G to T at 1885 | 12 | Glu to Stop at 585 |
| E588V | A to T at 1895 | 12 | Glu to Val at 588 |
| E608G | A to G at 1955 | 13 | Glu to Gly at 608 |
| E60K | G to A at 310 | 3 | Glu to Lys at 60 |
| E60X | G to T at 310 | 3 | Glu to Stop at 60 |
| E656X | G to T at 2098 | 13 | Glu to Stop at 656 |
| E664X | G to T at 2122 | 13 | Glu to Stop at 664 |
| E672del | deletion of 3 bp between 2145-2148 | 13 | deletion of Glu at 672 |
| E692X | G to T at 2206 | 13 | Glu to Stop at 692 |
| E725K | G to A at 2305 | 13 | Glu to Lys at 725 |
| E730X | G to T at 2320 | 13 | Glu to Stop at 730 |
| E7X | G to T at 151 | 1 | Glu to Stop at 7 |
| E822K | G to A at 2596 | 13 | Glu to Lys at 822 |
| E822X | G to T at 2596 | 13 | Glu to Stop at 822 |
| E823X | G to T at 2599 | 13 | Glu to Stop at 823 |
| E826K | G to A at 2608 | 13 | Glu to Lys at 826 |
| E827X | G to T at 2611 | 13 | Glu to Stop at 827 |
| E831X | G to T at 2623 | 14a | Glu to Stop at 831 |
| E92D | A to T at 408(GAA->GAT) | 4 | Gly to Asp at 92 |
| E92K | G to A at 406 | 4 | Glu to Lys at 92 |
| E92X | G to T at 406 | 4 | Glu to Stop at 92 |
| F1016S | T to C at 3179 | 17a | Phe to Ser at 1016 |
| F1052V | T to G at 3286 | 17b | Phe to Val at 1052 |
| F1074L | T to A at 3354 | 17b | Phe to Leu at 1074 |
| F1166C | T to G at 3629 | 19 | Phe to Cys at 1166 |
| F1257L | T to G at 3903 | 20 | Phe to Leu at 1257 |
| F1286S | T to C at 3989 | 20 | Phe to Ser at 1286 |
| F1300L | T to C at 4030 | 21 | Phe to Leu at 1300 |
| F1337V | T to G at 4141 | 22 | Phe to Val at 1337 (CBAVD) |
| F200I | T to A at 730 | 6a | Phe to Ile at 200 |
| F305V | T to G at 1045 | 7 | Phe 305 Val |
| F311L | C to G at 1065 | 7 | Phe to Leu at 311 |
| F316L | T to G at 1080 | 7 | Phe to Leu at 316 |
| F508C | T to G at 1655 | 10 | Phe to Cys at 508 |
| F508S | T to C at 1655 | 10 | Phe to Ser at 508 |
| F587I | T to A at 1891 | 12 | Phe to Ile at 587 |
| F693L(CTT) | T to C at 2209 | 13 | Phe to Leu at 693 |
| F693L(TTG) | T to G at 2211 | 13 | Phe to Leu at 693 |
| F87L | T to C at 391 | 3 | Phe to Leu at 87 |
| F932S | T to C at 2927 | 15 | Phe to Ser at 932 |
| F994C | T to G at 3113 | 16 | Phe to Cys at 994 |
| G1003E | G to A at 3140 | 17a | Gly to Glu at 1003 |
| G1003X | G to T at 3139 | 17a | Gly to Stop at 1003 |
| G103X | G to T at 439 | 4 | Gly to Stop at 103 |
| G1047D | G to A at 3272 | 17b | Gly to Asp at 1047 and mRNA |
| | | | splicing defect (CBAVD) |
| G1047R | G to C at 3271 | 17a | Gly to Arg at 1047 |
| G1061R | G to C at 3313 | 17b | Gly to Arg at 1061 |
| G1069R | G to A at 3337 | 17b | Gly to Arg at 1069 |
| G1123R | G to C at 3499 | 17b | Gly to Arg at 1123 mRNA |
| | | | splicing defect |
| G1127E | G to A at 3512 | 18 | Gly to Glu at 1127 |
| G1130A | G to C at 3521 | 18 | Gly to Ala at 1130 |
| G1237S | G to A at 3841 | 19 | Gly to Ser at 1237 |
| G1244E | G to A at 3863 | 20 | Gly to Glu at 1244 |
| G1244R | G to A at 3862 | 20 | Gly to Arg at 1244 |
| G1244V | G to T at 3863 | 20 | Gly to Val at 1244 |
| G1247R(G->A) | G to A at 3871 | 20 | Gly to Arg at 1247 |
| G1247R(G->C) | G to C at 3871 | 20 | Gly to Arg at 1247 |
| G1249E | G to A at 3878 | 20 | Gly to Glu at 1249 |
| G1249R | G to A at 3877 | 20 | Gly to Arg at 1249 |
| G126D | G to A at 509 | 4 | Gly to Asp at 126 |
| G1349D | G to A at 4178 | 22 | Gly to Asp at 1349 |
| G1349S | G to A at 4177 | 22 | Gly to Ser at 1349 |
| G149R | G to A at 577 | 4 | Gly to Arg at 149 |
| G149V | G to T at 578 | 4 | Gly to Val at 149 |
| G178E | G to A at 665 | 5 | Gly to Glu at 178 |
| G178R | G to A at 664 | 5 | Gly to Arg at 178 |
| G194R | G to A at 712 | 6a | Gly to Arg at 194 |
| G194V | G to T at 713 | 6a | Gly to Val at 194 |
| G213V | G to T at 771 | 6a | Gly to Val at 213 |
| G239R | G to A at 847 | 6a | Gly to Arg at 239 |
| G241R | G to A at 853 | 6a | Gly to Arg at 241 |
| G27E | G to A at 212 | 2 | Gly to Glu at 27 |
| G27R | G to A at 211 | 6b | Gly to Arg at 27 |
| G27R(211G to C) | G to C at 211 | 2 | Gly to Arg at 27 |
| G27X | G to T at 211 | 2 | Gly to Stop at 27 |
| G314E | G to A at 1073 | 7 | Gly to Glu at 314 |
| G314R | G to C at 1072 | 7 | Gly to Arg at 314 |
| G314V | G to T at 1073 | 7 | Gly to Val at 314 |
| G330X | G to T at 1120 | 7 | Gly to Stop at 330 |
| G424S | G to A at 1402 | 9 | Gly to Ser at 424 |
| G458V | G to T at 1505 | 9 | Gly to Val at 458 |
| G480C | G to T at 1570 | 10 | Gly to Cys at 480 |
| G480D | G to A at 1571 | 10 | Gly to Asp at 480 |
| G480S | G to A at 1570 | 10 | Gly to Ser at 480 |
| G486X | G to T at 1588 | 10 | Gly to Stop at 486 |
| G542X | G to T at 1756 | 11 | Gly to Stop at 542 |
| G544S | G to A at 1762 | 11 | Gly to Ser at 544 |
| G544V | G to T at 1763 | 11 | Gly to Val at 544 (CBAVD) |
| G550R | G to A at 1780 | 11 | Gly to Arg at 550 |
| G550X | G to T at 1780 | 11 | Gly to Stop at 550 |
| G551D | G to A at 1784 | 11 | Gly to Asp at 551 |
| G551S | G to A at 1783 | 11 | Gly to Ser at 551 |
| G576A | G to C at 1859 | 12 | Gly to Ala at 576 (CAVD) |
| G576X | G to T at 1858 | 12 | Gly to Stop at 576 |
| G622D | G to A at 1997 | 13 | Gly to Asp at 622 |
| | | | (oligospermia) |
| G628R(G->A) | G to A at 2014 | 13 | Gly to Arg at 628 |
| G628R(G->C) | G to C at 2014 | 13 | Gly to Arg at 628 |
| G673X | G to T at 2149 | 13 | Gly to Stop at 673 |
| G723V | G to T at 2300 | 13 | Gly to Val at 723 |
| G745X(Gly745X) | G to T at 2365 | 13 | Non-sense mutation |
| G85E | G to A at 386 | 3 | Gly to Glu at 85 |
| G85V | G to T at 386 | 3 | Gly to Val at 85 |
| G91R | G to A at 403 | 3 | Gly to Arg at 91 |
| G970D | G to A at 3041 | 16 | Gly to Asp at 970 |
| G970R | G to C at 3040 | 15 | Gly to Arg at 970 |
| G970S | G to A at 3040 | 15 | Gly to Ser at 970 |
| H1054D | C to G at 3292 | 17b | His to Asp at 1054 |
| H1054 L | A to T at 3293 | 17b | His to Leu at 1054 |
| H1054R | A to G at 3293 | 17b | His to Arg at 1054 |
| H1079P | A to C at 3368 | 17b | His to Pro at 1079 |
| H1085R | A to G at 3386 | 17b | His to Arg at 1085 |
| H1375P | A to C at 4256 | 22 | His to Pro at 1375 |
| H139L | A to T at 548 | 4 | His to Leu at 139 |
| H139R | A to G at 548 | 4 | His to Arg at 139 |
| H146R | A to G at 569 | 4 | His to Arg at 146 (CBAVD) |
| H199Q | T to G at 729 | 6a | His to Gln at 199 |
| H199R | A to G at 728 | 6a | His to Arg at 199 |
| H199Y | C to T at 727 | 6a | His to Tyr at 199 |
| H484R | A to G at 1583 | 10 | His to Arg at 484 |
| H484Y | C to T at 1582 | 10 | His to Tyr at 484 (CBAVD) |
| H609L | A to T at 1958 | 13 | His to Leu at 609 |
| H609R | A to G at 1958 | 13 | His to Arg at 609 |
| H620P | A to C at 1991 | 13 | His to Pro at 620 |
| H620Q | T to G at 1992 | 13 | His to Gln at 620 |
| H939D | C to G at 2947 | 15 | His to Asp at 939 |
| H939R | A to G at 2948 | 15 | His to Arg at 939 |
| H949L | A to T at 2978 | 15 | His to Leu at 949 |
| H949R | A to G at 2978 | 15 | His to Arg at 949 |
| H949Y | C to T at 2977 | 15 | His to Tyr at 949 |
| I1005R | T to G at 3146 | 17a | Ile to Arg at 1005 |
| I1027T | T to C at 3212 | 17a | Ile to Thr at 1027 |
| I1051V | A to G at 3283 | 17b | Ile to Val at 1051 |
| I105N | T to A at 446 | 4 | Ile to Asn at 105 |
| I1139V | A to G at 3547 | 18 | Ile to Val at 1139 |
| I119V | A to G at 487 | 4 | Iso to Val at 119 |
| I1230T | T to C at 3821 | 19 | Ile to Thr at 1230 |
| I1234L | A to C at 3832 | 19 | sequence variation |
| I1234V | A to G at 3832 | 19 | Ile to Val at 1234 |
| I125T | T to C at 506 | 4 | Ile to Thr at 125 |
| I1269N | T to A at 3938 | 20 | Ile to Asn at 1269 |
| I1328T | T to C at 4115 | 22 | Ile to Thr at 1328 |
| I132M | T to G at 528 | 4 | Ile to Met at 132 (sequence |
| | | | variation) |
| I1366T | T to C at 4229 | 22 | Ile to Thr at 1366 |
| I1398S | T to G at 4325 | 23 | Ile to Ser at 1398 |
| I148N | T to A at 575 | 4 | Ile to Asn at 148 |
| I148T | T to C at 575 | 4 | Ile to Thr at 148 |
| I175V | A to G at 655 | 5 | Ile to Val at 175 |
| I177T | T to C at 662 | 5 | Ile to Thr at 177 |
| I203M | C to G at 741 | 6a | Ile to Met at 203 |
| I285F | A to T at 985 | 6b | Ile to Phe at 285 |
| I331N | T to A at 1124 | 7 | Ile to Asn at 331 |
| I336K | T to A at 1139 | 7 | Ile to Lys at 336 |
| I340N | T to A at 1151 | 7 | Ile to Asn at 340 |
| I444S | T to G at 1463 | 9 | Ile to Ser at 444 |
| I444T | T to C at 1463 | 9 | Ile to Thr at 444 |
| I497V | A to G at 1621 | 10 | Ile to Val at 497 |
| I502N | T to A at 1637 | 10 | Ile to Asn at 502 |
| I502T | T to C at 1637 | 10 | Ile to Thr at 502 |
| I506L | A to C at 1648 | 10 | Ile to Leu at 506 |
| I506S | T to G at 1649 | 10 | Ile to Ser at 506 |
| I506T | T to C at 1649 | 10 | Ile to Thr at 506 |
| I506V (1648A/G) | A or G at 1648 | 10 | Ile or Val at 506 |
| I539T | T to C at 1748 | 11 | Ile to Thr at 539 |
| I556V | A to G at 1798 | 11 | Ile to Val at 556 (mutation) |
| I586V | A to G at 1888 | 12 | Ile to Val at 586 |
| I601F | A to T at 1933 | 13 | Ile to Phe at 601 |
| I618T | T to C at 1985 | 13 | Ile to Thr at 618 |
| I752S | T to G at 2387 (ATC->AGC) | 13 | Ileu to Ser at 752 |
| I807M | A or G at 2553 | 13 | sequence variation |
| I807V | A to G at 2551 | 13 | Ile to Val at 807 |
| I840T | T to C at 2651 | 14a | Ile to Thr at 840 |
| I918M | T to G at 2886 | 15 | Ile to Met at 918 |
| I980K | T to A at 3071 | 16 | Ile to Lys at 980 |
| I980M | A to G at 3072 | 16 | Ile to Met at 980 |
| I991V | A to G at 3103 | 16 | Ile to Val at 991 |
| IVS14a + 17del5 | 5 bp deletion between 2751 + 17 | intron | sequence variation |
| | and 2751 + 24 | 14a | |
| K1060T | A to C at 3311 | 17b | Lys to Thr at 1060 |
| K1080R | A to G at 3371 | 17b | Lys to Arg at 1080 |
| K114X | A to T at 472 | 4 | Lys to Stop at 114 |
| K1177R | A to G at 3662 | 19 | Lys to Arg at 1177 |
| K1177X | A to T at 3661 | 19 | Lys to Stop at 1177 |
| | | | (premature termination) |
| K1302R | A to G at 4037 (AAA->AGA) | 4 | Lys to Arg at 1302 |
| K1351E | A to G at 4183 | 22 | Lys to Glu at 1351 (CBAVD) |
| K14X | A to T at 172 | 1 | Lys to Stop at 14 |
| K162E | A to G at 616 | 4 | Lys to Glu at 162 |
| K166Q | A to G at 628 | 5 | Lys to Gln at 166 |
| K464N | G to T at 1524 | 9 | Lys to Asn at 464; mRNA |
| | | | splicing defect |
| K536X | A to T at 1738 | 11 | Lys to Stop codon at 536 |
| K598X | A to T at 1924 | 13 | Lys to Stop at 598 |
| K64E | A to G at 322 | 3 | Lys tu Glu at 64 |
| K683R | A to G at 2180 | 13 | Lys to Arg at 683 |
| K688X | A to T at 2194 | 13 | Lys to Stop at 688 |
| K68E | A to G at 334 | 3 | Lys to Glu at 68 |
| K68N | A to T at 336 | 3 | Lys to Asn at 68 |
| K710X | A to T at 2260 | 13 | Lys to Stop at 710 |
| K716X | AA to GT at 2277 and 2278 | 13 | Lys to Stop at 716 |
| K830X | A to T at 2620 | 13 | Lys to Stop at 830 |
| K946X | A to T at 2968 | 15 | Lys to Stop at 946 |
| L101S | T to C at 434 | 4 | Leu to Ser at 101 |
| L101X | T to G at 434 | 4 | Leu to Stop at 101 |
| L102P | T to C at 437 | 4 | Leu to Pro at 102 |
| L102R | T to G at 437 | 4 | Leu to Arg at 102 |
| L1059L (3309A/G) | A or G at 3309 | 17b | sequence variation |
| L1059X | T to G at 3308 | 17b | Leu to Stop at 1059 |
| L1065F | C to T at 3325 | 17b | Leu to Phe at 1065 |
| L1065P | T to C at 3326 | 17b | Leu to Pro at 1065 |
| L1065R | T to G at 3326 | 17b | Leu to Arg at 1065 |
| L1077P | T to C at 3362 | 17b | Leu to Pro at 1077 |
| L1093P | T to C at 3410 | 17b | Leu to Pro at 1093 |
| L1096R | T to G at 3419 | 17b | Leu to Arg at 1096 |
| L1156F | G to T at 3600 | 18 | Leu to Phe at 1156 |
| L1227S | T to C at 3812 | 19 | Leu to Ser at 1227 |
| L1254X | T to G at 3893 | 20 | Leu to Stop at 1254 |
| L1260R | T to G at 3911 | 20 | Leu to Arg at 1260 |
| L127X | T to G at 512 | 4 | Leu to Stop at 127 |
| L130V | C to G at 520 | 4 | Leucine to Valine at 130 |
| L1324P | T to C at 4103 | 22 | Leu to Pro at 1324 |
| L1335F | C to T at 4135 | 22 | Leu to Phe at 1335 |
| L1335P | T to C at 4136 | 22 | Leu to Pro at 1335 |
| L1339F | C to T at 4147 | 22 | Leu to Phe at 1339 |
| L137H | T to A at 542 | 4 | Leu to His at 137 |
| L137P | T to C at 542 | 4 | Leu to Pro at 137 (sequence |
| | | | variation) |
| L137R | T to G at 542 | 4 | Leu to Arg at 137 |
| L1388Q | T to A at 4295 | 23 | Leu to Gln at 1388 (CBAVD) |
| L1388V | C to G at 4294 | 23 | Leu to Val at 1388 |
| L138ins | insertion of CTA, TAC or ACT at | 4 | insertion of leucine at 138 |
| | nucleotide 544, 545 or 546 | | |
| L1414S | T to C at 4373 | 23 | Leu to Ser at 1414 |
| L145H | T to A at 566 | 4 | Leu to His at 145 |
| L1480P | T to C at 4571 | 24 | Leu to Pro a 1480 |
| L159S | T to C at 608 | 4 | Leu to Ser at 159 |
| L159X | T to A at 608 | 4 | Leu to Stop at 159 |
| L15P | T to C at 176 | 1 | Leu to Pro at 15 |
| L165S | T to C at 626 | 5 | Leu to Ser at 165 |
| L183I | C to A at 679 | 5 | Leu to Ile at 183 |
| L206F | G to T at 750 | 6a | Leu to Phe at 206 |
| L206W | T to G at 749 | 6a | Leu to Trp at 206 |
| L210P | T to C at 761 | 6a | Leu to Pro at 210 |
| L218X | T to A at 785 | 6a | Leu to Stop at 218 |
| L227R | T to G at 812 | 6a | Leu to Arg at 227 |
| L24F | G to C at 204 | 2 | Leu to Phe at 24 |
| L293M | C to A at 1009 | 7 | Leu to Met at 293 |
| L320F | A to T at 1092 | 7 | Leu to Phe at 320 |
| L320V | T to G at 1090 | 7 | Leu to Val at 320 CAVD |
| L320X | T to A at 1091 | 7 | Leu to Stop at 320 |
| L327R | T to G at 1112 | 7 | Leu to Arg at 327 |
| L346P | T to C at 1169 | 7 | Leu to Pro at 346 |
| L365P | T to C at 1226 | 7 | Leu to Pro at 365 |
| L375F | A to C at 1257 | 8 | Leu to Phe at 375 (CUAVD) |
| L383L (1281G/A) | G or A at 1281 | 8 | sequence variation |
| L383S | T to C at 1280 | 8 | Leu to Ser at 383 |
| L468P | T to C at 1535 | 10 | Leu to Pro at 468 |
| L548Q | T to A at 1775 | 11 | Leu to Gln at 548 |
| L558S | T to C at 1805 | 11 | Leu to Ser at 558 |
| L568F | G to T at 1836 | 12 | Leu to Phe at 568 (CBAVD) |
| L568X | T to A at 1835 | 12 | Leu to Stop at 568 |
| L571S | T to C at 1844 | 12 | Leu to Ser at 571 |
| L594P | T to C at 1913 | 13 | Leu to Pro at 594 |
| L610S | T to C at 1961 | 13 | Leu to Ser at 610 |
| L619S | T to C at 1988 | 13 | Leu to Ser at 619 |
| L61P | T to C at 314 | 3 | Leucine to Proline at position |
| | | | 61 |
| L633I | C to A at 2029 | 13 | Leu to Ile at 633 |
| L633P | T to C at 2030 | 13 | Leu to Pro at 633 |
| L636P | T to C at 2039 | 13 | Leu to Pro at 636 |
| L719X | T to A at 2288 | 13 | Leu to Stop at 719 |
| L732X | T to G at 2327 | 13 | Leu to Stop at 732 |
| L829L (2619A/G) | A or G at 2619 | 13 | sequence variation |
| L867X | T to A at 2732 | 14a | Leu to Stop at 867 |
| L88S | T to C at 395 | 3 | Leu to Ser at 88 |
| L88X(T->A) | T to A at 395 | 3 | Leu to Stop at 88 |
| L88X(T->G) | T to G at 395 | 3 | Leu to Stop at 88 |
| L90S | T to C at 401 | 3 | Leu to Ser at 90 |
| L927P | T to C at 2912 | 15 | Leu to Pro at 927 |
| L967S | T to C at 3032 | 15 | Leu to Ser at 967 |
| | | | (oligospermia) |
| L973F | TC to AT at 3048 and 3049 | 16 | Leu to Phe at 973 (CBAVD) |
| L973H | T to A at 3050 | 16 | Leu to His at 973 |
| L973P | T to C at 3050 | 16 | Leu to Pro at 973 |
| L997F | G or C at 3123 | 17a | Leu or Phe at 997 (sequence |
| | | | variation) |
| M1028I | G to T at 3216 | 17a | Met to Ile at 1028 |
| M1028R | T to G at 3215 | 17a | Met to Arg at 1028 |
| M1101K | T to A at 3434 | 17b | Met to Lys at 1101 |
| M1101R | T to G at 3434 | 17b | Met to Arg at 1101 |
| M1105R | T to G at 3446 | 17b | Met to Arg at 1105 |
| M1137R | T to G at 3542 | 18 | Met to Arg at 1137 |
| M1137T | T to C at 3542 | 18 | Met to Thr at 1137 |
| M1137V | A to G at 3541 | 18 | Met to Val at 1137 |
| M1140K | T to A at 3551 | 18 | Met to Lys at 1140 |
| M1210I | G to A at 3762 | 19 | Met to Ile at 1210 |
| M1210K | T to A at 3761 | 19 | Met to Lys at 1210 |
| M1407T | T to C at 4352 | 23 | Met to Thr at 1407 |
| M152L | A to T at 586 | 4 | Met to Leu at 152 |
| M152R | T to G at 587 | 4 | Met to Arg at 152 |
| M152V | A to G at 586 | 4 | Met to Val at 152 (mutation) |
| M1I(ATA) | G to A at 135 | 1 | no translation initiation |
| M1I(ATT) | G to T at 135 | 1 | no translation initiation |
| M1K | T to A at 134 | 1 | no translation initiation |
| M1L | A to C at 133 | 1 | Met to Leu at 1 |
| M1T | T to C at 134 | 1 | Met to Thr at 1 |
| M1V | A to G at 133 | 1 | no translation initiation |
| M243L | A to C at 859 | 6a | Met to Leu at 243 (ATG to |
| | | | CTG) |
| M244K | T to A at 863 | 6a | Met to Lys at 244 |
| M265R | T to G at 926 | 6b | Met to Arg at 265 |
| M281T | T to C at 974 | 6b | Met to Thr at 281 |
| M348K | T to A at 1175 | 7 | Met to Lys at 348 |
| M348T | T to C at 1175 | 7 | Met to Thr at 348 |
| M348V | A to G at 1174 | 7 | Met to Val at AS 348 |
| M394R | T to G at 1313 | 8 | Met to Arg at 394 |
| M469V | A to G 1537 | 10 | Met to Val at 469 |
| M470V | A or G at 1540 | 10 | sequence variation |
| M498I | G to C at 1626 | 10 | Met (ATG) to Ileu (ATC) at |
| | | | 498 |
| M595I | G to A at 1917 | 13 | Met to Ile at 595 |
| M595T | T to C at 1916 | 13 | Met to Thr at 595 |
| M82V | A to G at 376 | 3 | Met to Val at 82 |
| M952I | G to C at 2988 | 15 | Met to Ile at 952 CBAVD |
| | | | mutation |
| M952T | T to C at 2987 | 15 | Met to Thr at 952 |
| M961I | G to T at 3015 | 15 | Met to Ile at 961 |
| N1088D | A to G at 3394 | 17b | Asn to Asp at 1088 |
| N113I | A to T at 470 | 4 | Asn to Ile |
| N1148K | C to A at 3576 | 18 | Asn to Lys at 1148 |
| N1148S | A to G at 3575 | 18 | Asn to Ser at 1148 |
| N1195T | A to C at 3716 | 19 | Asn to Thr at 1195 |
| N1303H | A to C at 4039 | 21 | Asn to His at 1303 |
| N1303I | A to T at 4040 | 21 | Asn to Ile at 1303 |
| N1303K | C to G at 4041 | 21 | Asn to Lys at 1303 |
| N1432K | C to G at 4428 | 24 | sequence variation |
| N186K | C to A at 690 | 5 | Asn to Lys at 186 |
| N187K | C to A at 693 | 5 | Asn to Lys at 187 |
| N189K | C to A at 699 | 5 | Asn to Lys at 189 |
| N189S | A to G at 698 | 5 | Asn to Ser at 189 |
| N287Y | A to T at 991 | 6b | Asn to Tyr at 287 |
| N369Y | A to T at 1318 | 8 | Asn to Tyr at 396 |
| N416S | A to G at 1379 | 9 | Asn to Ser at 416 |
| N418S | A to G at 1385 | 9 | Asn to Ser at 418 |
| N66S | A to G at 329 | 3 | Asn to Ser at 66 |
| N782K | C to A at 2478 | 13 | Asn to Lys at 782 |
| N900T | A to C at 2831 | 15 | Asn to Thr at 900 |
| P1013H | C to A at 3170 | 17a | Pro to His at 1013 |
| P1013L | C to T at 3170 | 17a | Pro to Leu at 1013 |
| P1021A | C to G at 3193 | 17a | Pro to Ala at 1021 |
| P1021S | C to T at 3193 | 17a | Pro to Ser at 1021 (CBAVD) |
| P1072L | C to T at 3347 | 17b | Pro to Leu at 1072 |
| P111A | C to G at 463 | 4 | Pro to Ala at 111 |
| P111L | C to T at 464 | 4 | Pro to Leu at 111 |
| P1290P (4002A/G) | A or G at 4002 | 20 | sequence variation |
| P1290S | C to T at 4000 | 20 | Pro to Ser at 1290 |
| P1290T | C to A at 4000 | 20 | Pro to Thr at 1290 |
| P1306P (4050C/T) | C or T at 4050 | 21 | sequence variation |
| P1372 L | C to T at 4247 | 22 | Pro to Leu at 1732 |
| P1372T | C to A 4246 | 22 | Pro to Thr at 1372 |
| P140L | C to T at 551 | 4 | Pro to Leu at 140 |
| P140S | C to T at 550 | 4 | Pro to Ser at 140 |
| P205R | C to G at 746 | 6a | Pro to Arg at 205 |
| P205S | C to T at 745 | 6a | Pro to Ser at 205 |
| P324L | C to T at 1103 | 7 | Pro to Leu at 324 |
| P355S | C to T at 1195 | 7 | Pro to Ser at 355 |
| P439S | C to T at 1447 | 9 | Pro to Ser at 439 |
| P499A | C to G at 1627 | 10 | Pro to Ala at 499 (CBAVD) |
| P574H | C to A at 1853 | 12 | Pro to His at 574 |
| P574S | C to T at 1852 | 12 | Pro to Ser at 574 |
| P5L | C to T at 146 | 1 | Pro to Leu at 5 |
| P67L | C to T at 332 | 3 | Pro to Leu at 67 |
| P750L | C to T at 2381 | 13 | Pro to Leu at 750 |
| P841R | C to G at 2654 | 14a | Pro to Arg at 841 |
| P99L | C to T at 428 | 4 | Pro to Leu at 99 |
| poly-T tract variations | variable number (5T, 7T, 9T) of | intron 8 | sequence variation (3 variants |
| | thymidines at the poly-T tract | | of which IVS8-5T is affecting |
| | starting at position 1342-6 | | splicing of exon 9) |
| Q1035X | C to T at 3235 | 17a | Nonsense mutation |
| Q1042X | C to T at 3256 | 17a | Gln to Stop at 1042 |
| Q1071H | G to T at 3345 | 17b | Gln to His at 1071 |
| Q1071P | A to C at 3344 | 17b | Gln to Pro at 1071 |
| Q1071X | C to T at 3343 | 17b | Gln to Stop at 1071 |
| Q1100P | A to C at 3431 | 17b | Gln to Pro at 1100 |
| Q1144X | C to T at 3562 | 18 | Gln to Stop at 1144 |
| Q1186Q (3690A/G) | A or G at 3690 | 19 | sequence variation |
| Q1186X | C to T at 3688 | 19 | Gln to Stop at 1186 |
| Q1238R | A to G at 3845 | 19 | Gln to Arg at 1238 |
| Q1238X | C to T at 3844 | 19 | Gln to Stop at 1238 |
| Q1268R | A to G at 3935 | 20 | Gln to Arg at 1268 |
| Q1281X | C to T at 3973 | 20 | Gln to Stop at 1281 |
| Q1291H | G to C at 4005 | 20 | Gln to His at 1291; mRNA |
| | | | splicing defect |
| Q1291R | A to G at 4004 | 20 | Gln to Arg at 1291 |
| Q1291X | C to T at 4003 | 20 | Gln to Stop at 1291 |
| Q1309H | G to T at 4059 | 21 | Gln to His at 1309 |
| Q1313K | C to A at 4069 | 21 | Gln to Lys at 1313 |
| Q1313X | C to T at 4069 | 21 | Gln to Stop at 1313 |
| Q1352E | C to G at 4186 | 22 | Gln to Glu at 1352 |
| Q1352H(G->C) | G to C at 4188 | 22 | Gln to His at 1352 |
| Q1352H(G->T) | G to T at 4188 | 22 | Gln to His at 1352 |
| Q1382X | C to T at 4276 | 23 | Gln to Stop at 1382 |
| Q1390X | 4300C > T | 23 | Gln to stop at 1390 |
| Q1411X | C to T at 4363 | 23 | Gln to Stop at 1411 |
| Q1412X | C to T at 4366 | 23 | Gln to Stop at 1412 |
| Q1463H | G to T at 4521 | 24 | Gln to His a 1463 |
| Q1476X | C to T at 4558 | 24 | Gln to Stop at 1476 |
| Q151K | C to A at 583 (CAG->AAG) | 4 | Gln to Lys at 151 |
| Q151X | C to T at 583 | 4 | Gln to Stop at 151 |
| Q179K | C to A at 667 | 5 | Gln to Lys at 179 |
| Q207X | C to T at 751 | 6a | Gln to Stop at 207 |
| Q220R | A to G at 791 | 6a | Gln to Arg at 220 |
| Q220X | C to T at 790 | 6a | Gln to Stop at 220 |
| Q237E | C to G at 841 | 6a | Gln to Glu at 237 |
| Q290X | C to T at 1000 | 6b | Gln to Stop at 290 |
| Q2X (together with | C to T at 136 and A to T at 139 | 1 | Gln to Stop at codon 2 and |
| R3W) | | | Arg to Trp at codon 3 |
| Q30X | C to T at 220 | 2 | Gln to Stop at 30 |
| Q353H | A to C at 1191 | 7 | Gln to His at 353 |
| Q353X | C to T at 1189 | 7 | Gln to Stop at 353 |
| Q359K/T360K | C to A at 1207 and C to A at 1211 | 7 | Glu to Lys at 359 and Thr to |
| | | | Lys at 360 |
| Q359R | A to G at 1208 | 7 | Gln to Arg at 359 |
| Q378R | A to G at 1265 | 8 | Gln to Arg at 378 |
| Q39X | C to T at 247 | 2 | Gln to Stop at 39 |
| Q414X | C to T at 1372 | 9 | Gln to Stop at 414 |
| Q452P | A to C at 1487 | 9 | Gln to Pro at 452 |
| Q493P | A to C at 1610 | 10 | Gln to Pro at 493 |
| Q493R | A to G at 1610 | 10 | Gln to Arg at 493 |
| Q493X | C to T at 1609 | 10 | Gln to Stop at 493 |
| Q525X | C to T at 1705 | 10 | Gln to Stop at 525 |
| Q552K | C to A at 1786 | 11 | Gln to Lys at 552 |
| Q552X | C to T at 1786 | 11 | Gln to Stop at 552 |
| Q634X | C to T at 2032 | 13 | Gln to Stop at 634 |
| Q637X | C to T at 2041 | 13 | Gln to Stop at 637 |
| Q685X | C to T at 2185 | 13 | Gln to Stop at 685 |
| Q689X | C to T at 2197 | 13 | Gln to Stop at 689 |
| Q715X | C to T at 2275 | 13 | Gln to Stop at 715 |
| Q720X | C to T at 2290 | 13 | Gln to Stop at 720 |
| Q781X | C to T at 2473 | 13 | Gln to Stop at 781 |
| Q814X | C to T at 2572 | 13 | Gln to Stop at 814 |
| Q890R | A to G at 2801 | 15 | Gln to Arg at 890 |
| Q890X | C to T at 2800 | 15 | Gln to Stop at 890 |
| Q98P | A to C at 425 | 4 | Gln to Pro at 98 |
| Q98R | A to G at 425 | 4 | Gln to Arg at 98 |
| Q98X | C to T at 424 | 4 | Gln to Stop at 98 (Pakistani |
| | | | specific) |
| R1048G | A to G at 3274 | 17b | Arg to Gly a 1048 |
| R1066C | C to T at 3328 | 17b | Arg to Cys at 1066 |
| R1066H | G to A at 3329 | 17b | Arg to His at 1066 |
| R1066L | G to T at 3329 | 17b | Arg to Leu at 1066 |
| R1066S | C to A at 3328 | 17b | Arg to Ser at 1066 |
| R1070P | G to C at 3341 | 17b | Arg to Pro at 1070 |
| R1070Q | G to A at 3341 | 17b | Arg to Gln at 1070 |
| R1070W | C to T at 3340 | 17b | Arg to Trp at 1070 |
| R1102X | A to T at 3436 | 17b | Arg to Stop at 1102 |
| R1128X | A to T at 3514 | 18 | Arg to Stop at 1128 |
| R1158X | C to T at 3604 | 19 | Arg to Stop at 1158 |
| R1162X | C to T at 3616 | 19 | Arg to Stop at 1162 |
| R117C | C to T at 481 | 4 | Arg to Cys at 117 |
| R117G | C to G at 481 | 4 | Arg to Gly at 117 |
| R117H | G to A at 482 | 4 | Arg to His at 117 |
| R117L | G to T at 482 | 4 | Arg to Leu at 117 |
| R117P | G to C at 482 | 4 | Arg to Pro at 117 |
| R1239S | G to C at 3849 | 19 | Arginine to Serine at 1239 |
| R1283K | G to A at 3980 | 20 | Arg to Lys at 1283 |
| R1283M | G to T at 3980 | 20 | Arg to Met at 1283 |
| R1358S | A to T at 4206 | 22 | Arg to Ser at 1358 |
| R1422W | C to T at 4396 | 24 | Arg to Trp at 1422 |
| R1438W | C to T at 4444 | 24 | Arg to Try at 1438 |
| R1453W | C to T at 4489 | 24 | Arg to Trp at 1453 |
| R170C | C to T at 640 | 5 | Arg to Cys at 170 |
| R170G | C to G at 640 | 5 | Arg to Gly at 170 |
| R170H | G to A at 641 | 5 | Arg to His at 170 |
| R248T | G to C at 875 | 6a | Arg to Thr at 248 (CBAVD) |
| R258G | A to G at 904 | 6b | Arg to Gly at 258 |
| R297Q | G to A at 1022 | 7 | Arg to Gln at 297 |
| R297W | C to T at 1021 | 7 | Arg to Trp at 297 |
| R31C | C to T at 223 | 2 | Arg to Cys at 31 |
| R31L | G to T at 224 | 2 | Arg to Leu at 31 |
| R334L | G to T at 1133 | 7 | Arg to Leu at 334 |
| R334Q | G to A at 1133 | 7 | Arg to Gln at 334 |
| R334W | C to T at 1132 | 7 | Arg to Trp at 334 |
| R347C | C to T at 1171 | 7 | Arg to Cys at 347 |
| R347H | G to A at 1172 | 7 | Arg to His at 347 |
| R347L | G to T at 1172 | 7 | Arg to Leu at 347 |
| R347P | G to C at 1172 | 7 | Arg to Pro at 347 |
| R352G | C to G at 1186 | 7 | Arg to Gly at 352 |
| R352Q | G to A at 1187 | 7 | Arg to Gln at 352 |
| R352W | C to T at 1186 | 7 | Arg to Trp at 352 |
| R516G | A to G at 1678 | 10 | Arg to Gly at 516 |
| R553G | C to G at 1789 | 11 | Arg to Gly at 553 |
| R553Q | G to A at 1790 | 11 | Arg to Gln at 553 (associated |
| | | | with [delta]F508; |
| R553X | C to T at 1789 | 11 | Arg to Stop at 553 |
| R555G | A to G at 1795 | 11 | Arg to Gly at 555 |
| R55K | G to A at 296 | 2 | Arg to Lys at 55 |
| R560G | A to G at 1810 | 11 | Ala to Gly at 560 |
| R560K | G to A at 1811 | 11 | Arg to Lys at 560 |
| R560S | A to C at 1812 | 12 | Arg to Ser at 560 |
| R560T | G to C at 1811 | 11 | Arg to Thr at 560; mRNA |
| | | | splicing defect |
| R600G | A to G at 1930 | 13 | Arg to Gly at 600 |
| R668C | C or T at 2134 | 13 | sequence variation |
| R709Q | G to A at 2258 | 13 | Arg to Gln at 709 |
| R709X | C to T at 2257 | 13 | Arg to Stop at 709 |
| R735K | G to A at 2336 | 13 | Arg to Lys at 735 |
| R74Q | G to A at 353 | 3 | Arg to Gln at 74 |
| R74W | C to T at 352 | 3 | Arg to Trp at 74 |
| R751P | G to C at 2384 | 13 | Arg to Pro at 751 |
| R75L | G to T at 356 | 3 | Arg to Leu at 75 |
| R75Q | G or A at 356 | 3 | sequence variation |
| R75X | C to T at 355 | 3 | Arg to Stop at 75 |
| R764X | C to T at 2422 | 13 | Arg to Stop at 764 |
| R766M | G to T at 2429 | 13 | Arg to Met at 766 |
| R785X | C to T at 2485 | 13 | Arg to Stop at 785 |
| R792G | C to G at 2506 | 13 | Arg to Gly at 792 |
| R792X | C to T at 2506 | 13 | Arg to Stop at 792 |
| R810G | A to G at 2560 | 13 | Arg to Gly at 810 |
| R851L | G to T at 2684 | 14a | Arg to Leu at 851 |
| R851X | C to T at 2683 | 14a | Arg to Stop at 851 |
| R933G | A to G at 2929 | 15 | Arg to Gly at 933 |
| R933S | A to T at 2931 | 15 | Arg to Ser at 933 (CBAVD) |
| S108F | C to T at 455 | 4 | Ser to Phe at 108 |
| S10R | A to C at 160 | 1 | Ser to Arg at 10 |
| S1118C | C to G at 3485 | 17b | Ser to Cys at 1118 |
| S1118F | C to T at 3485 | 17b | Ser to Phe at 1118 |
| S1159F | C to T at 3608 | 19 | Ser to Phe at 1159 |
| S1159P | T to C at 3607 | 19 | Ser to Pro at 1159 |
| S1161R | A to C at 3613 or C to G at 3615 | 19 | Ser to Arg at 1161 |
| S1196X | C to G at 3719 | 19 | Ser to Stop at 1196 |
| S1206X | C to G at 3749 | 19 | Ser to Stop at 1206 |
| S1206X(C > A) | C to A at 3749 | 19 | Ser to Stop at 1206 |
| S1235R | T to G at 3837 | 19 | Ser to Arg at 1235 |
| S1251N | G to A 3884 | 20 | Ser to Asn at 1251 |
| S1255L | C to T at 3896 | 20 | Ser to Leu at 1255 |
| S1255P | T to C at 3895 | 20 | Ser to Pro at 1255 |
| S1255X | C to A at 3896 and A to G at 3739 | 20 | Ser to Stop at 1255 and Ile to |
| | in exon 19 | | Val at 1203 |
| S1311R | A to C at 4063 or T to A or G at | 21 | Ser to Arg at 1311 |
| | 4065 | | |
| S13F | C to T at 170 | 1 | Ser to Phe at 13 |
| S1426F | C to T at 4409 | 24 | Ser to Phe at 1426 |
| S1426P | T to C at 4408 | 24 | Ser to Pro at 1426 |
| S1455X | C to G at 4496 | 24 | Ser to Stop at 1455 |
| S158N | G to A at 605 | 4 | Ser to Asn at 158 |
| S158R | A to C at 604 | 4 | Ser to Arg at 158 |
| S158T | G to C at 605 | 4 | Ser to Thr at 158 |
| S18G | A to G at 184 | 1 | Ser to Gly at 18 |
| S307N | G to A at 1052 | 7 | Ser to Asn at 307 |
| S313X | C to A at 1070 | 7 | Ser to Stop |
| S321P | T to C at 1093 | 7 | Ser to Pro at 321 |
| S341P | T to C at 1153 | 7 | Ser to Pro at 341 |
| S364P | T to C at 1222 | 7 | Ser to Pro at 364 |
| S42F | C to T at 257 | 2 | Ser to Phe at 42 |
| S431G | A to G at 1423 | 9 | Ser to Gly a 431 |
| S434X | C to G at 1433 | 9 | Ser to Stop at 434 |
| S466L | C to T at 1529 | 10 | Ser to Leu at 466 (CBAVD) |
| S466X(TAA) | C to A at 1529 | 10 | Ser to Stop at 466 |
| S466X(TAG) | C to G at 1529 | 10 | Ser to Stop at 466 |
| S485C | A to T at 1585 | 10 | Ser to Cys at 485 |
| S489X | C to A at 1598 | 10 | Ser to Stop at 489 |
| S492F | C to T at 1607 | 10 | Ser to Phe at 492 |
| S4X | C to A at 143 | 1 | Ser to Stop at 4 |
| S50P | T to C at 280 | 2 | Ser to Pro at 50 |
| S50Y | C to A at 281 | 2 | Ser to Tyr at 50 (CBAVD) |
| S519G | A to G at 1687 | 10 | Ser to Gly at 519 |
| S549I | G to T at 1778 | 11 | Ser to Ile at 549 |
| S549N | G to A at 1778 | 11 | Ser to Asn at 549 |
| S549R(A->C) | A to C at 1777 | 11 | Ser to Arg at 549 |
| S549R(T->G) | T to G at 1779 | 11 | Ser to Arg at 549 |
| S573C | C to G at 1850 | 12 | Ser to Cys at 573 |
| S589I | G to T at 1898 | 12 | Ser to Ile at 589 (splicing) |
| S589N | G to A at 1898 | 12 | Ser to Asn at 589 (mRNA |
| | | | splicing defect) |
| S660T | T to A at 2110 | 13 | Ser to Thr a 660 |
| S686Y | C to A at 2189 | 13 | Ser to Tyr at 686 |
| S712C | C to G at 2267 | 13 | Ser to Cys at 712 |
| S737F | C to T at 2342 | 13 | missense |
| S737F | C to T at 2342 | 13 | Ser to Phe at 737 |
| S753R | C to G at position 2391 | 13 | Serine to arginine at 753 |
| S776X | C to G at 2459 | 13 | Ser to Stop at 776 |
| S813P | T to C at 2569 | 13 | Ser to Pro at 813 |
| S895T | G to C at 2816 | 15 | Ser to Thr at 895 |
| S902R | C to G at 2838 | 15 | Ser to Arg at 902 |
| S911R | A to C at 2863 or T to A or T to G | 15 | Ser to Arg at 911 |
| | at 2865 | | |
| S912L | C to T at 2867 | 15 | Ser to Leu at 912 |
| S912X | C to A at 2867 | 15 | Ser to Stop at 912 |
| S945L | C to T at 2966 | 15 | Ser to Leu at 945 |
| S977F | C to T at 3062 | 16 | Ser to Phe at 977 |
| S977P | T to C at 3061 | 16 | Ser to Pro at 977 |
| T1053I | C to T at 3290 | 17b | Thr to Ile at 1053 (CBAVD) |
| T1057A | A to G at 3301 | 17b | Thr to Ala at 1057 |
| T1086A | A to G at 3388 | 17b | Thr to Ala at 1086 |
| T1086I | C to T at 3389 | 17b | Thr to Ile at 1086 |
| T1142I | C to T at 3557 | 18 | Thr to Ile at 1142 |
| T1246I | C to T at 3869 | 20 | Thr to Ile at 1246 (mutation) |
| T1252P | A to C at 3886 | 20 | Thr to Pro at 1252 |
| T1263A | A to G at 3919 | 20 | Thr to Ala at 1263 |
| T1263I | C to T at 3920 | 20 | Thr to Ile at 1263 |
| T1299I | C to T at 4028 | 21 | Thr to Ile at 1299 |
| T338A | A to G at 1144 | 7 | Thr to Ala at 338 |
| T338I | C to T at 1145 | 7 | Thr to Ile at 338 |
| T351I | C to T at 1184 | 7 | Thr to Ile at 351 |
| T351S | C or G at 1184 | 7 | sequence variation |
| T360R | C to G at 1211 | 7 | sequence variation (Thr to Arg |
| | | | at 360) |
| T388M | C to T at 1295 | 8 | Thr to Met at 388 (sequence |
| | | | variation) |
| T388X | AC to TA at 1294 | 8 | Thr to Stop at 388 |
| T501A | A to G at 1633 | 10 | Thr to Ala at 501 |
| T582I | C to T at 1877 | 12 | Thr to Ile at 582 |
| T582R | C to G at 1877 | 12 | Thr to Arg at 582 |
| T582S | A to T at 1876 | 12 | Thr to Ser at 582 |
| T599T (1929T/A) | T or A at 1929 | 13 | sequence variation |
| T604I | C to T at 1943 | 13 | Thr to Ile at 604 |
| T604S | C to G at 1943 | 13 | Thr to Ser at 604 |
| T665S | A to T at 2125 | 13 | Thr to Ser at 665 |
| T760M | C to T at 2411 | 13 | Thr to Met at 760 |
| T788I | C to T at 2495 | 13 | Thr to Ile at 788 |
| T896I | C to T at 2819 | 15 | Thr to Ile at 896 |
| T908N | C to A at 2855 | 15 | Thr to Asn at 908 |
| TAAA repeats | 9 or 11 repeats of TAAA (SEQ ID | intron 9 | sequence variation |
| | NO: 12) at | | |
| TTGA repeats | 5-7 copies of repeat at around | intron 6a | sequence variation |
| | 876-31 | | |
| V1008D | T to A at 3155 | 17a | Val to Asp at 1008 |
| V1020E | T to A at 3191 | 17a | Val to Glu at 1020 |
| V1108L | G to C at 3454 | 17b | Val to Leu at 1108 |
| V1129G | 3518T > G | 18 | Val to Gly at 1129 |
| V1147I | G to A at 3571 | 18 | Val to Ile at 1147 |
| V1153E | T to A at 3590 | 18 | Val to Glu at 1153 (CBAVD) |
| V1190D | T to A at 3701 | 19 | Val to Asp at 1190 |
| V1212I | G to A at 3766 | 19 | Val to Ile at 1212 |
| V1240G | T to G at 3851 | 20 | Val to Gly at 1240 |
| V1293I | G to A at 4009 | 21 | Val to Ile at 1293 |
| V1318A | T to C at 4085 | 21 | Val to Ala at 1318 |
| V1397E | T to A at 4322 | 23 | Val to Glu at 1397 |
| V201M | G to A at 733 | 6a | Val to Met at 201 |
| V232D | T to A at 827 | 6a | Val to Asp at 232 (CBAVD) |
| V317A | T to C at 1082 | 7 | Val to Ala at 317 |
| V322A | T to C at 1097 | 7 | Val to Ala at 322 (mutation) |
| V322M (1096(G/A)) | G or A at 1096 | 7 | sequence variation |
| V392A | T to C at 1307 | 8 | Val to Ala at 392 CAVD |
| V392G | T to G at 1307 | 8 | Val to Gly at 392 |
| V456A | T to C at 1499 | 9 | Val to Ala at 456 (sequence |
| | | | variation) |
| V456F | G to T at 1498 | 9 | Val to Phe at 456 |
| V520F | G to T at 1690 | 10 | Val to Phe at 520 |
| V520I | G to A at 1690 | 10 | Val to Ile at 520 |
| V562I | G to A at 1816 | 12 | Val to Ile at 562 |
| V562L | G to C at 1816 | 12 | Val to Leu at 562 |
| V603F | G to T at 1939 | 13 | Val to Phe at 603 |
| V754M | G to A at 2392 | 13 | Val to Met at 754 |
| V855I | G to A at 2695 | 14a | Val to Ile at 855 (sequence |
| | | | variation) |
| V920L | G to T at 2890 | 15 | Val to Leu at 920 |
| V920M | G to A at 2890 | 15 | Val to Met at 920 |
| V922L | G to C at 2896 | 15 | Val to Leu at 922 |
| V938G | T to G at 2945 | 15 | Val to Gly at 938 (CAVD) |
| V938L | G to C at 2944 | 15 | Val to Leu at 938 |
| W1063X | G to A at 3321 | 17b | Trp to Stop at 1063 |
| W1089X | G to A at 3398 | 17b | Trp to Stop at 1089 |
| W1098L | G to T at 3425 | 17b | Trp to Leu at 1098 |
| W1098R | T to C at 3424 | 17b | Trp to Arg at 1098 |
| W1098X(TAG) | G to A at 3425 | 17b | Trp to Stop at 1098 |
| W1098X(TGA) | G to A at 3426 | 17b | Trp to Stop at 1098 |
| W1145X | G to A at 3567 | 18 | Trp to Stop at 1154 |
| W1204X(3743G->A) | G to A at 3743 | 19 | Trp to Stop at 1204 |
| W1204X(3744G->A) | G to A at 3744 | 19 | Trp to Stop at 1204 |
| W1274X | G to A at 3954 | 20 | Trp to Stop at 1274 |
| W1282C | G to T at 3978 | 20 | Trp to Cys at 1282 |
| W1282G | T to G at 3976 | 20 | Trp to Gly at 1282 |
| W1282R | T to C at 3976 | 20 | Trp to Arg at 1282 |
| W1282X | G to A at 3978 | 20 | Trp to Stop at 1282 |
| W1310X | G to A at 4061 | 21 | Trp to Stop at 1310 |
| W1316X | G to A at 4079 | 21 | Trp to Stop at 1316 |
| W19C | G to T at 189 | 2 | Trp to Cys at 19 |
| W19X | G to A at 189 | 2 | Trp to Stop at 19 |
| W202X | G to A at 738 | 6a | Try to Stop at 202 |
| W216C | G to T at 780 | 6a | Trp to Cys at 216 |
| W216X | G to A at 779 | 6a | Trp to Stop at 216 |
| W277R | T to A at 961 | 6b | Trp to Arg at 277 |
| W356S | G to C at 1199 | 7 | Tryptophan to Serine at codon |
| | | | 356 |
| W356X | G to A at 1200 | 7 | Trp to Stop at 356 |
| W361R(T->A) | T to A at 1213 | 7 | Trp to Arg at 361 |
| W361R(T->C) | T to C at 1213 | 7 | Trp to Arg at 361 |
| W401X(TAG) | G to A at 1334 | 8 | Trp to Stop at 401 |
| W401X(TGA) | G to A at 1335 | 8 | Trp to Stop at 401 |
| W496X | G to A at 1619 | 10 | Trp to Stop at 496 |
| W57G | T to G at 301 | 3 | Trp to Gly at 57 |
| W57R | T to C at 301 | 3 | Trp to Arg at 57 |
| W57X(TAG) | G to A at 302 | 3 | Trp to Stop at 57 |
| W57X(TGA) | G to A at 303 | 3 | Trp to Stop at 57 |
| W679X | G to A at 2168 | 13 | Trp to Stop at 679 |
| W79R | T to C at 367 | 3 | Trp to Arg at 79 |
| W79X | G to A at 368 | 3 | Trp to Stop at 79 |
| W846X | G to A at 2669 | 14a | Trp to Stop at 846 |
| W846X | G to A at 2670 | 14a | Trp to Stop at 846 |
| (2670TGG > TGA) | | | |
| W882X | G to A at 2777 | 14b | Trp to Stop at 882 |
| Y1014C | A to G at 3173 | 17a | Tyr to Cys at 1014 |
| Y1032C | A to G at 3227 | 17a | Tyr to Cys at 1032 (CBAVD) |
| Y1032N | T to A at 3226 | 17a | Tyr to Asn at 1032 |
| Y1073C | A to G at 3350 | 17b | Tyr to Cys at 1073 |
| Y1092C | A to G at 3407 | 17b | Tyr to Cys at 1092 |
| Y1092H | T to C at 3406 | 17b | Tyr to His at 1092 |
| Y1092X(C->A) | C to A at 3408 | 17b | Tyr to Stop at 1092 |
| Y1092X(C->G) | C to G at 3408 | 17b | Tyr to Stop at 1092 |
| Y109C | A to G at 458 | 4 | Tyr to Cys at 109 |
| Y109N | T to A at 457 | 4 | Tyr to Asn at 109 |
| Y109X | T to A at 459 | 4 | Tyr to Stop at 109 |
| Y1182X | C to G at 3678 | 19 | Tyr to Stop at 1182 |
| Y122C | A to G at 497 | 4 | Tyr to Cys at 122 |
| Y122H | T to C at 496 | 4 | Tyr to His at 122 |
| Y122X | T to A at 498 | 4 | Tyr to Stop at 122 |
| Y1307C | A to G at 4052 | 21 | Tyr to Cys at 1307 |
| Y1307X | T to A at 4053 | 21 | Tyr to Stop at 1307 |
| Y1381H | T to C at 4273 | 23 | Tyr to His at 1381 |
| Y1381X | C to A at 4275 | 23 | Tyr to Stop at 1381 |
| Y161D | T to G at 613 | 4 | Tyr to Asp at 161 |
| Y161N | T to A at 613 | 4 | Tyr to Asn at 161 |
| Y161S | A to C at 614 (together with | 4 | Tyr to Ser at 161 |
| | 612T/A) | | |
| Y247X | C to G at 873 | 6a | Tyr to Stop at 247 |
| Y301C | A to G at 1034 | 7 | Tyr to Cys at 301 |
| Y304X | C to G at 1044 | 7 | Tyr to Stop at 304 |
| Y515H | T to C at 1675 | 10 | Tyr to His at 515 |
| Y517C | A to G at 1682 | 10 | Tyr to Cys at 517 |
| Y563C | A to G at 1820 | 12 | Tyr to Cys at 563 |
| Y563D | T to G at 1819 | 12 | Tyr to Asp at 563 |
| Y563N | T to A at 1819 | 12 | Tyr to Asn at 563 |
| Y569C | A to G at 1838 | 12 | Tyr to Cys at 569 |
| Y569D | T to G at 1837 | 12 | Tyr to Asp at 569 |
| Y569H | T to C at 1837 | 12 | Tyr to His at 569 |
| Y569X | T to A at 1839 | 12 | Tyr to Stop at 569 |
| Y577F | A to T at 1862 | 12 | Tyr to Phe at 577 |
| Y577Y (1863C/T) | C or T at 1863 | 12 | sequence variation (Tyr at 577 |
| | | | no change) |
| Y849X | C to A at 2679 | 14a | Tyr to Stop at 849 |
| Y84H | T to C at 382 | 3 | Tyr to His at 84 |
| Y852X | T to G at 2688 | 14a | Tyr to Stop at 852 (Premature |
| | | | termination) |
| Y89C | A to G at 398 | 3 | Tyr to Cys at 89 |
| Y913C | A to G at 2870 | 15 | Tyr to Cys at 913 |
| Y913X | T to A at 2871 | 15 | Tyr to Stop at 913 |
| Y914C | A to G at 2873 | 15 | Tyr to Cys at 914 |
| Y917C | A to G at 2882 | 15 | Tyr to Cys at 917 |
| Y917D | T to G at 2881 | 15 | Tyr to Asp at 917 |
| Y919C | A to G at 2888 | 15 | Tyr to Cys at 919 |

*Unless otherwise indicated, the numbers listed herein refer to exon numbers.

TABLE 2
CFTR Mutants and Their Disease Association.

| | | | SWISS-PROT |
|---|---|---|---|
| | Length | | Feature Table |
| Position(s) | (aa) | Description and disease association | Identifier |
| 31 | 1 | R → L in CF. Ref.44 | VAR_000103 |
| 42 | 1 | S → F in CF. Ref.48 | VAR_000104 |
| 44 | 1 | D → G in CF. | VAR_000105 |
| 50 | 1 | S → Y in CBAVD. Ref.54 | VAR_000107 |
| 57 | 1 | W → G in CF. Ref.42 | VAR_000108 |
| 67 | 1 | P → L in CF. | VAR_000109 |
| 74 | 1 | R → W in CF. | VAR_000110 |
| 85 | 1 | G → E in CF. Ref.58 | VAR_000112 |
| 87 | 1 | F → L in CF. Ref.39 | VAR_000113 |
| 91 | 1 | G → R in CF. | VAR_000114 |
| 92 | 1 | E → K in CF. Ref.26 Ref.29 | VAR_000115 |
| 98 | 1 | Q → R in CF. Ref.46 | VAR_000116 |
| 105 | 1 | I → S in CF. | VAR_000117 |
| 109 | 1 | Y → C in CF. Ref.37 | VAR_000118 |
| 110 | 1 | D → H in CF. | VAR_000119 |
| 111 | 1 | P → L in CBAVD. Ref.69 | VAR_000120 |
| 117 | 1 | R → C in CF. Ref.26 Ref.48 | VAR_000121 |
| | | Ref.58 Ref.65 | |
| 117 | 1 | R → H in CF and CBAVD. | VAR_000122 |
| 117 | 1 | R → L in CF. Ref.26 Ref.48 | VAR_000123 |
| | | Ref.58 Ref.65 | |
| 117 | 1 | R → P in CF. Ref.26 Ref.48 | VAR_000124 |
| | | Ref.58 Ref.65 | |
| 120 | 1 | A → T in CF. Ref.38 | VAR_000125 |
| 139 | 1 | H → R in CF. Ref.48 | VAR_000126 |
| 141 | 1 | A → D in CF. Ref.56 | VAR_000127 |
| 148 | 1 | I → T in CF. dbSNP rs35516286. | VAR_000128 |
| 149 | 1 | G → R in CBAVD. Ref.40 | VAR_000129 |
| 178 | 1 | G → R in CF. | VAR_000130 |
| 192 | 1 | Missing in CF. Ref.65 | VAR_000131 |
| 193 | 1 | E → K in CBAVD and CF. | VAR_000132 |
| 199 | 1 | H → Q in CF. Ref.34 | VAR_000133 |
| 199 | 1 | H → Y in CF. Ref.34 | VAR_000134 |
| 205 | 1 | P → S in CF. Ref.30 | VAR_000135 |
| 206 | 1 | L → W in CF. Ref.43 | VAR_000136 |
| 225 | 1 | C → R in CF. | VAR_000137 |
| 244 | 1 | M → K in CBAVD. Ref.69 | VAR_000138 |
| 258 | 1 | R → G in CBAVD. Ref.40 | VAR_000139 |
| 287 | 1 | N → Y in CF. Ref.58 | VAR_000140 |
| 297 | 1 | R → Q in CF. | VAR_000141 |
| 301 | 1 | Y → C in CF. | VAR_000142 |
| 307 | 1 | S → N in CF. | VAR_000143 |
| 311 | 1 | F → L in CF. Ref.59 | VAR_000144 |
| 311 | 1 | Missing in CF. Ref.59 | VAR_000145 |
| 314 | 1 | G → E in CF. Ref.50 | VAR_000146 |
| 314 | 1 | G → R in CF. Ref.50 | VAR_000147 |
| 334 | 1 | R → W in CF; mild. | VAR_000148 |
| 336 | 1 | I → K in CF. | VAR_000150 |
| 338 | 1 | T → I in CF; mild; isolated | VAR_000151 |
| | | hypotonic dehydration. | |
| | | Ref.47 Ref.64 | |
| 346 | 1 | L → P in CF; dominant mutation | VAR_000152 |
| | | but mild phenotype. Ref.33 | |
| 347 | 1 | R → H in CF. | VAR_000153 |
| 347 | 1 | R → L in CF. | VAR_000154 |
| 347 | 1 | R → P in CF; MILD. | VAR_000155 |
| 352 | 1 | R → Q in CF. | VAR_000156 |
| 359 | 1 | Q → K in CF. | VAR_000157 |
| 359-360 | 2 | QT → KK in CF. | VAR_000158 |
| 370 | 1 | K → KNK in CF. | VAR_000159 |
| 455 | 1 | A → E in CF. Ref.58 | VAR_000160 |
| 456 | 1 | V → F in CF. | VAR_000161 |
| 458 | 1 | G → V in CF. | VAR_000162 |
| 480 | 1 | G → C in CF. | VAR_000165 |
| 492 | 1 | S → F in CF. | VAR_000166 |
| 504 | 1 | E → Q in CF. | VAR_000167 |
| 507 | 1 | Missing in CF. | VAR_000170 |
| 508 | 1 | Missing in CF and CBAVD; | VAR_000171 |
| | | most common mutation; 72% | |
| | | of the CF patients; CFTR | |
| | | fails to be properly delivered | |
| | | to plasma membrane. | |
| 513 | 1 | D → G in CBAVD. Ref.68 | VAR_000173 |
| 520 | 1 | V → F in CF. Ref.23 | VAR_000174 |
| 544 | 1 | G → V in CBAVD. Ref.69 | VAR_000175 |
| 549 | 1 | S → N in CF. | VAR_000176 |
| 549 | 1 | S → I in CF. | VAR_000177 |
| 549 | 1 | S → R in CF. | VAR_000178 |
| 551 | 1 | G → D in CF. Ref.58 | VAR_000179 |
| 551 | 1 | G → S in CF. Ref.58 | VAR_000180 |
| 553 | 1 | R → Q in CF. | VAR_000181 |
| 558 | 1 | L → S in CF. | VAR_000182 |
| 559 | 1 | A → T in CF. | VAR_000183 |
| 560 | 1 | R → K in CF. Ref.63 | VAR_000184 |
| 560 | 1 | R → S in CF. Ref.63 | VAR_000185 |
| 560 | 1 | R → T in CF. Ref.63 | VAR_000186 |
| 562 | 1 | V → L in CF. Ref.53 | VAR_000188 |
| 563 | 1 | Y → N in CF. | VAR_000189 |
| 569 | 1 | Y → C in CF. Ref.51 Ref.63 | VAR_000190 |
| 569 | 1 | Y → D in CF. Ref.51 Ref.63 | VAR_000191 |
| 569 | 1 | Y → H in CF. Ref.51 Ref.63 | VAR_000192 |
| 571 | 1 | L → S in CF. | VAR_000193 |
| 572 | 1 | D → N in CF. Ref.45 | VAR_000194 |
| 574 | 1 | P → H in CF. | VAR_000195 |
| 579 | 1 | D → G in CF. Ref.42 Ref.70 | VAR_000197 |
| 601 | 1 | I → F in CF. | VAR_000198 |
| 610 | 1 | L → S in CF. | VAR_000199 |
| 613 | 1 | A → T in CF. | VAR_000200 |
| 614 | 1 | D → G in CF. | VAR_000201 |
| 618 | 1 | I → T in CF. | VAR_000202 |
| 619 | 1 | L → S in CF. Ref.34 | VAR_000203 |
| 620 | 1 | H → P in CF. | VAR_000204 |
| 620 | 1 | H → Q in CF. | VAR_000205 |
| 622 | 1 | G → D in oligospermia. | VAR_000206 |
| 628 | 1 | G → R in CF. | VAR_000207 |
| 633 | 1 | L → P in CF. | VAR_000208 |
| 648 | 1 | D → V in CF. | VAR_000209 |
| 651 | 1 | D → N in CF. | VAR_000210 |
| 665 | 1 | T → S in CF. Ref.49 | VAR_000211 |
| 754 | 1 | V → M in CF. | VAR_000214 |
| 766 | 1 | R → M in CBAVD. | VAR_000215 |
| 792 | 1 | R → G in CBAVD. | VAR_000216 |
| 800 | 1 | A → G in CBAVD. Ref.40 | VAR_000217 |
| 807 | 1 | I → M in CBAVD. dbSNP | VAR_000218 |
| | | rs1800103. | |
| 822 | 1 | E → K in CF. | VAR_000219 |
| 826 | 1 | E → K in thoracic sarcoidosis. | VAR_000220 |
| 866 | 1 | C → Y in CF. | VAR_000221 |
| 912 | 1 | S → L Ref.32 | VAR_000222 |
| 913 | 1 | Y → C in CF. | VAR_000223 |
| 917 | 1 | Y → C in CF. | VAR_000224 |
| 949 | 1 | H → Y in CF. Ref.32 | VAR_000225 |
| 952 | 1 | M → I in CF. | VAR_000226 |
| 997 | 1 | L → F in CF. dbSNP rs1800111. | VAR_000227 |
| 1005 | 1 | I → R in CF. Ref.34 | VAR_000228 |
| 1006 | 1 | A → E in CF. Ref.48 | VAR_000229 |
| 1013 | 1 | P → L in CF. Ref.60 | VAR_000230 |
| 1028 | 1 | M → I in CF. Ref.60 | VAR_000231 |
| 1052 | 1 | F → V in CF. Ref.28 | VAR_000232 |
| 1061 | 1 | G → R in CF. Ref.28 Ref.52 | VAR_000233 |
| 1065 | 1 | L → P in CF. Ref.32 Ref.66 | VAR_000234 |
| 1065 | 1 | L → R in CF. Ref.32 Ref.66 | VAR_000235 |
| 1066 | 1 | R → C in CF. Ref.28 Ref.57 | VAR_000236 |
| 1066 | 1 | R → H in CF. Ref.28 Ref.57 | VAR_000237 |
| 1066 | 1 | R → L in CF. Ref.28 Ref.57 | VAR_000238 |
| 1067 | 1 | A → T in CF. | VAR_000239 |
| 1070 | 1 | R → Q in CF. Ref.28 Ref.58 | VAR_000241 |
| 1070 | 1 | R → P in CF. Ref.28 Ref.58 | VAR_000242 |
| 1071 | 1 | Q → P in CF. Ref.32 | VAR_000243 |
| 1072 | 1 | P → L in CF. | VAR_000244 |
| 1077 | 1 | L → P in CF. | VAR_000245 |
| 1085 | 1 | H → R in CF. Ref.28 | VAR_000246 |
| 1098 | 1 | W → R in CF. Ref.44 | VAR_000247 |
| 1101 | 1 | M → K in CF. Ref.27 Ref.28 | VAR_000248 |
| 1137 | 1 | M → V in CF. | VAR_000249 |
| 1140 | 1 | Missing in CF. Ref.55 | VAR_000250 |
| 1152 | 1 | D → H in CF. | VAR_000251 |
| 1234 | 1 | I → V in CF. | VAR_000254 |
| 1235 | 1 | S → R in CF. | VAR_000255 |
| 1244 | 1 | G → E in CF. | VAR_000256 |
| 1249 | 1 | G → E in CF. Ref.35 | VAR_000257 |
| 1251 | 1 | S → N in CF. | VAR_000258 |
| 1255 | 1 | S → P in CF. Ref.25 | VAR_000259 |
| 1270 | 1 | D → N in CF. dbSNP rs11971167. | VAR_000260 |
| 1282 | 1 | W → R in CF. | VAR_000261 |
| 1283 | 1 | R → M in CF. Ref.24 | VAR_000262 |
| 1286 | 1 | F → S in CF. | VAR_000263 |
| 1291 | 1 | Q → H in CF. Ref.23 Ref.34 | VAR_000264 |
| 1291 | 1 | Q → R in CF. Ref.23 Ref.34 | VAR_000265 |
| 1303 | 1 | N → H in CF. Ref.58 | VAR_000266 |
| 1303 | 1 | N → K in CF. Ref.58 | VAR_000267 |
| 1349 | 1 | G → D in CF. | VAR_000268 |
| 1364 | 1 | A → V in CBAVD. Ref.69 | VAR_000269 |
| 1397 | 1 | V → E in CF. Ref.36 | VAR_000270 |
| 1070 | 1 | R → W in CBAVD. | VAR_011564 |
| 1101 | 1 | M → R in CF. Ref.27 Ref.28 | VAR_011565 |

(CF: cystic fibrosis; CBAVD: congenital bilateral absence of the vas deferens.)
References in Table 2:
[1] Riordan J. R. etal., Science 245: 1066-1073(1989) [PubMed: 2475911]
[2] Zielenski J. etal., Genomics 10: 214-228(1991) [PubMed: 1710598]
[3] Stacy R. etal., Submitted (JAN-2006) to the EMBL/GenBank/DDBJ databases
[4] Hillier L. W. etal., Nature 424: 157-164(2003) [PubMed: 12853948]
[5] Scherer S. W. etal., Science 300: 767-772(2003) [PubMed: 12690205]
[6] Picciotto M. R. etal., J. Biol. Chem. 267: 12742-12752(1992) [PubMed: 1377674]
[7] Chang X.-B. etal., J. Biol. Chem. 269: 18572-18575(1994) [PubMed: 7518437]
[8] Neville D. C. A. etal., Protein Sci. 6: 2436-2445(1997) [PubMed: 9385646]
[9] Pagani F. etal., J. Biol. Chem. 275: 21041-21047(2000) [PubMed: 10766763]
[10] Cheng J. etal., J. Biol. Chem. 277: 3520-3529(2002) [PubMed: 11707463]
[11] Park M. etal., J. Biol. Chem. 277: 50503-50509(2002) [PubMed: 12403779]
[12] Swiatecka-Urban A. etal., J. Biol. Chem. 279: 38025-38031(2004) [PubMed: 15247260]
[13] McIntosh I. etal., FASEB J. 6: 2775-2782(1992) [PubMed: 1378801]
[14] Aznarez I. etal., Hum. Mol. Genet. 12: 2031-2040(2003) [PubMed: 12913074]
[15] Chappe V. etal., J. Physiol. (Lond.) 548: 39-52(2003) [PubMed: 12588899]
[16] Wang Y. etal., EMBO J. 25: 5058-5070(2006) [PubMed: 17053785]
[17] Hoedemaeker F. J. etal., Proteins 30: 275-286(1998) [PubMed: 9517543]
[18] Karthikeyan S. etal., J. Biol. Chem. 276: 19683-19686(2001) [PubMed: 11304524]
[19] Tsui L.-C. etal., Hum. Mutat. 1: 197-203(1992) [PubMed: 1284534]
[20] Cutting G. R. etal., Nature 346: 366-369(1990) [PubMed: 1695717]
[21] Kerem B.-S. etal., Proc. Natl. Acad. Sci. U.S.A. 87: 8447-8451(1990) [PubMed: 2236053]
[22] Dolganov G. etal., Genomics 10: 266-269(1991) [PubMed: 1710600]
[23] Jones C. T. etal., Hum. Mol. Genet. 1: 11-17(1992) [PubMed: 1284466]
[24] Cheadle J. P. etal., Hum. Mol. Genet. 1: 123-125(1992) [PubMed: 1284468]
[25] Lissens W. etal., Hum. Mol. Genet. 1: 441-442(1992) [PubMed: 1284530]
[26] Shackleton S. etal., Hum. Mol. Genet. 1: 439-440(1992) [PubMed: 1284529]
[27] Zielenski J. etal., Am. J. Hum. Genet. 52: 609-615(1993) [PubMed: 7680525]
[28] Mercier B. etal., Genomics 16: 296-297(1993) [PubMed: 7683628]
[29] Nunes V. etal., Hum. Mol. Genet. 2: 79-80(1993) [PubMed: 7683954]
[30] Chillon M. etal., Hum. Mol. Genet. 2: 1741-1742(1993) [PubMed: 7505694]
[31] Gasparini P. etal., Hum. Mutat. 2: 389-394(1993) [PubMed: 7504969]
[32] Ghaneb N. etal., Genomics 21: 434-436(1994) [PubMed: 7522211]
[33] Boteva K. etal., Hum. Genet. 93: 529-532(1994) [PubMed: 7513296]
[34] Doerk T. etal., Hum. Genet. 94: 533-542(1994) [PubMed: 7525450]
[35] Greil I. etal., Hum. Hered. 44: 238-240(1994) [PubMed: 7520022]
[36] Petreska L. etal., Hum. Mol. Genet. 3: 999-1000(1994) [PubMed: 7524913]
[37] Schaedel C. etal., Hum. Mol. Genet. 3: 1001-1002(1994) [PubMed: 7524909]
[38] Chillon M. etal., Hum. Mutat. 3: 223-230(1994) [PubMed: 7517264]
[39] Bienvenu T. etal., Hum. Mutat. 3: 395-396(1994) [PubMed: 8081395]
[40] Mercier B. etal., Am. J. Hum. Genet. 56: 272-277(1995) [PubMed: 7529962]
[41] Jezequel P. etal., Clin. Chem. 41: 833-835(1995) [PubMed: 7539342]
[42] Brancolini V. etal., Hum. Genet. 96: 312-318(1995) [PubMed: 7544319]
[43] Desgeorges M. etal., Hum. Genet. 96: 717-720(1995) [PubMed: 8522333]
[44] Zielenski J. etal., Hum. Mutat. 5: 43-47(1995) [PubMed: 7537150]
[45] Verlingue C. etal., Hum. Mutat. 5: 205-209(1995) [PubMed: 7541273]
[46] Romey M.-C. etal., Hum. Mutat. 6: 190-191(1995) [PubMed: 7581407]
[47] Leoni G. B. etal., J. Pediatr. 127: 281-283(1995) [PubMed: 7543567]
[48] Ferec C. etal., Mol. Cell. Probes 9: 135-137(1995) [PubMed: 7541510]
[49] Messaoud T. etal., Eur. J. Hum. Genet. 4: 20-24(1996) [PubMed: 8800923]
[50] Nasr S. Z. etal., Hum. Mutat. 7: 151-154(1996) [PubMed: 8829633]
[51] Petreska L. etal., Hum. Mutat. 7: 374-375(1996) [PubMed: 8723693]
[52] Bienvenu T. etal., Hum. Mutat. 7: 376-377(1996) [PubMed: 8723695]
[53] Hughes D. J. etal., Hum. Mutat. 8: 340-347(1996) [PubMed: 8956039]
[54] Zielenski J. etal., Hum. Mutat. 9: 183-184(1997) [PubMed: 9067761]
[55] Clavel C. etal., Hum. Mutat. 9: 368-369(1997) [PubMed: 9101301]
[56] Gouya L. etal., Hum. Mutat. 10: 86-87(1997) [PubMed: 9222768]
[57] Casals T. etal., Hum. Mutat. 10: 387-392(1997) [PubMed: 9375855]
[58] Shrimpton A. E. etal., Hum. Mutat. 10: 436-442(1997) [PubMed: 9401006]
[59] Friedman K. J. etal., Am. J. Hum. Genet. 62: 195-196(1998) [PubMed: 9443874]
[60] Onay T. etal., Hum. Genet. 102: 224-230(1998) [PubMed: 9521595]
[61] Bombieri C., Hum. Genet. 103: 718-722(1998) [PubMed: 9921909]
[62] Vankeerberghen A. etal., Hum. Mol. Genet. 7: 1761-1769(1998) [PubMed: 9736778]
[63] Malone G. etal., Hum. Mutat. 11: 152-157(1998) [PubMed: 9482579]
[64] Leoni G. B. etal., Hum. Mutat. 11: 337-337(1998) [PubMed: 9554753
[65] Feldmann D. etal., Hum. Mutat. Suppl. 1: S78-S80(1998) [PubMed: 9452048]
[66] Casals T. etal., Hum. Mutat. Suppl. 1: S99-S102(1998) [PubMed: 9452054]
[67] Shackleton S. etal., Hum. Mutat. Suppl. 1: S156-S157(1998) [PubMed: 9452073]
[68] Bienvenu T. etal., Hum. Mutat. 12: 213-214(1998) [PubMed: 10651488]
[69] de Meeus A. etal., Hum. Mutat. 12: 480-480(1998)
[70] Picci L. etal., Hum. Mutat. 13: 173-173(1999) [PubMed: 10094564]

In other aspects, the invention provides methods of making and using the novel cells and cell lines expressing CFTR (e.g., wild type or mutant CFTR). In other aspects, the cells and cell lines of the invention can be used to screen for modulators of CFTR function, including modulators that are specific for a particular form (e.g., mutant form) of CFTR, e.g., modulators that affect CFTR's chloride ion conductance function or CFTR's response to forskolin. These modulators are useful as therapeutics that target, for example, mutant CFTRs in disease states or tissues. CFTR-associated diseases and conditions include, without limitation, cystic fibrosis, lung diseases (e.g., chronic obstructive pulmonary and pulmonary edema), gastrointestinal conditions (e.g., CF pathologies, bowel cleaning, irritable bowel syndrome, constipation, diarrhea, cholera, viral gastroenteritis, malabsorption syndromes, and short bowel syndrome), endocrinal conditions (e.g., pancreatic dysfunction in CF patients), infertility (e.g., sperm motility and sperm capacitation problems and hostile cervical mucus), dry mouth, dry eye, glaucoma, and other deficiencies in regulation of mucosal and/or epithelial fluid absorption and secretion.

In various embodiments, the cell or cell line of the invention expresses CFTR at a consistent level of expression for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200 days or over 200 days, where consistent expression refers to a level of expression that does not vary by more than: 1%, 2%, 3%, 4%, 5%, 6%, 7%, 8% 9% or 10% over 2 to 4 days of continuous cell culture; 2%, 4%, 6%, 8%, 10% or 12% over 5 to 15 days of continuous cell culture; 2%, 4%, 6%, 8%, 10%, 12%, 14%, 16%, 18% or 20% over 16 to 20 days of continuous cell culture; 2%, 4%, 6%, 8%, 10%, 12%, 14%, 16%, 18%, 20%, 22%, 24% over 21 to 30 days of continuous cell culture; 2%, 4%, 6%, 8%, 10%, 12%, 14%, 16%, 18%, 20%, 22%, 24%, 26%, 28% or 30% over 30 to 40 days of continuous cell culture; 2%, 4%, 6%, 8%, 10%, 12%, 14%, 16%, 18%, 20%, 22%, 24%, 26%, 28% or 30% over 41 to 45 days of continuous cell culture; 2%, 4%, 6%, 8%, 10%, 12%, 14%, 16%, 18%, 20%, 22%, 24%, 26%, 28% or 30% over 45 to 50 days of continuous cell culture; 2%, 4%, 6%, 8%, 10%, 12%, 14%, 16%, 18%, 20%, 22%, 24%, 26%, 28%, 30% or 35% over 45 to 50 days of continuous cell culture, 2%, 4%, 6%, 8%, 10%, 12%, 14%, 16%, 18%, 20%, 22%, 24%, 26%, 28% or 30% over 50 to 55 days of continuous cell culture, 2%, 4%, 6%, 8%, 10%, 12%, 14%, 16%, 18%, 20%, 22%, 24%, 26%, 28%, 30% or 35% over 50 to 55 days of continuous cell culture; 1%, 2%, 3%, 4%, 5%, 10%, 15%, 20%, 25%, 30%, 35% or 40% over 55 to 75 days of continuous cell culture;1%, 2%, 3%, 4%, 5%, 6%, 10%, 15%, 20%, 25%, 30%, 35%, 40% or 45% over 75 to 100 days of continuous cell culture; 1%, 2%, 3%, 4%, 5%, 6%, 10%, 15%, 20%, 25%, 30%, 35%, 40% or 45% over 101 to 125 days of continuous cell culture; 1%, 2%, 3%, 4%, 5%, 6%, 10%, 15%, 20%, 25%, 30%, 35%, 40% or 45% over 126 to 150 days of continuous cell culture; 1%, 2%, 3%, 4%, 5%, 6%, 10%, 15%, 20%, 25%, 30%, 35%, 40% or 45% over 151 to 175 days of continuous cell culture; 1%, 2%, 3%, 4%, 5%, 6%, 10%, 15%, 20%, 25%, 30%, 35%, 40% or 45% over 176 to 200 days of continuous cell ulture; 1%, 2%, 3%, 4%, 5%, 6%, 10%, 15%, 20%, 25%, 30%, 35%, 40% or 45% over more than 200 days of continuous cell culture.

In some embodiments, the cells and cell lines of the invention express a CFTR wherein one or more physiological properties of the cells/cell lines remain(s) substantially constant over time. A physiological property includes any observable, detectable or measurable property of cells or cell lines apart from the expression of the CFTR.

In some embodiments, the expression of CFTR can alter one or more physiological properties. Alteration of a physiological property includes any change of the physiological property due to the expression of CFTR, e.g., a stimulation, activation, or increase of the physiological property, or an inhibition, blocking, or decrease of the physiological property. In these embodiments, the one or more constant physiological properties can indicate that the functional expression of the CFTR also remains constant.

The invention provides a method for culturing a plurality of cells or cell lines expressing a CFTR under constant culture conditions, wherein cells or cell lines can be selected that have one or more desired properties, such as stable expression of a CFTR and/or one or more substantially constant physiological properties.

In some embodiments where a physiological property can be measured, the physiological property is determined as an average of the physiological property measured in a plurality of cells or a plurality of cells of a cell line. In certain embodiments, a physiological property is measured over at least 10; 100; 1,000; 10,000; 100,000; 1,000,000; or at least 10,000,000 cells and the average remains substantially constant over time. In some embodiments, the average of a physiological property is determined by measuring the physiological property in a plurality of cells or a plurality of cells of a cell line wherein the cells are at different stages of the cell cycle. In other embodiments, the cells are synchronized with respect to cell cycle.

In some embodiments, a physiological property is observed, detected, measured or monitored on a single cell level. In certain embodiments, the physiological property remains substantially constant over time on a single cell level.

In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 12 hours. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 1 day. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 2 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 5 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 10 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 20 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 30 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 40 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 50 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 60 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 70 days. In certain embodiments, a physiological property remains substantially constant over time if it does not vary by more than 0.1%, 0.5%, 1%, 2.5%, 5%, 10%, 15%, 20%, 25%;30%, 35%, 40%, 45%, or 50% over 80 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over 90 days. In certain embodiments, a physiological property remains substantially constant over time if it does not vary by more than 0.1%, 0.5%, 1%, 2.5%, 5%, 10%, 15%, 20%, 25%, 30%, 35%, 40%, 45%, or 50% over the course of 1 passage, 2 passages, 3 passages, 5 passages, 10 passages, 25 passages, 50 passages, or 100 passages.

Examples of cell physiological properties include, but are not limited to: growth rate, size, shape, morphology, volume; profile or content of DNA, RNA, protein, lipid, ion, carbohydrate or water; endogenous, engineered, introduced, gene-activated or total gene, RNA or protein expression or content; propensity or adaptability to growth in adherent, suspension, serum-containing, serum-free, animal-component free, shaken, stationary or bioreactor growth conditions; propensity or adaptability to growth in or on chips, arrays, microarrays, slides, dishes, plates, multiwell plates, high density multiwell plates, flasks, roller bottles, bags or tanks; propensity or adaptability to growth using manual or automated or robotic cell culture methodologies; abundance, level, number, amount or composition of at least one cell organelle, compartment or membrane, including, but not limited to cytoplasm, nucleoli, nucleus, ribosomes, rough endoplasmic reticulum, Golgi apparatus, cytoskeleton, smooth endoplasmic reticulum, mitochondria, vacuole, cytosol, lysosome, centrioles, chloroplasts, cell membrane, plasma cell membrane, nuclear membrane, nuclear envelope, vesicles (e.g., secretory vesicles), or membrane of at least one organelle; having acquired or having the capacity or propensity to acquire at least one functional or gene expression profile (of one or more genes) shared by one or more specific cell types or differentiated, undifferentiated or dedifferentiated cell types, including, but not limited to: a stem cell, a pluripotent cell, an omnipotent cell or a specialized or tissue specific cell including one of the liver, lung, skin, muscle (including but not limited to: cardiac muscle, skeletal muscle, striatal muscle), pancreas, brain, testis, ovary, blood, immune system, nervous system, bone, cardiovascular system, central nervous system, gastro-intestinal tract, stomach, thyroid, tongue, gall bladder, kidney, nose, eye, nail, hair, taste bud cell or taste cell, neuron, skin, pancreas, blood, immune, red blood cell, white blood cell, killer T-cell, enteroendocrine cell, secretory cell, kidney, epithelial cell, endothelial cell, a human, animal or plant cell; ability to or capacity to uptake natural or synthetic chemicals or molecules including, but not limited to: nucleic acids, RNA, DNA, protein, small molecules, probes, dyes, oligonucleotides (including modified oligonucleotides) or fluorogenic oligonucleotides; resistance to or capacity to resist negative or deleterious effects of chemicals or substances that negatively affect cell growth, function or viability, including, but not limited to: resistance to infection, drugs, chemicals, pathogens, detergents, UV, adverse conditions, cold, hot, extreme temperatures, shaking, perturbation, vortexing, lack of or low levels of oxygen, lack of or low levels of nutrients, toxins, venoms, viruses or compound, treatment or agent that has an adverse effect on cells or cell growth; suitability for use in in vitro tests, cell based assays, biochemical or biological tests, implantation, cell therapy or secondary assays, including, but not limited to: large scale cell culture, miniaturized cell culture, automated cell culture, robotic cell culture, standardized cell culture, drug discovery, high throughput screening, cell based assay, functional cell based assay (including but not limited to membrane potential assays, calcium flux assays, reporter assays, G-protein reporter assays), ELISA, in vitro assays, in vivo applications, secondary testing, compound testing, binding assays, panning assays, antibody panning assays, phage display, imaging studies, microscopic imaging assays, immunofluorescence studies, RNA, DNA, protein or biologic production or purification, vaccine development, cell therapy, implantation into an organism, animal, human or plant, isolation of factors secreted by the cell, preparation of cDNA libraries, or infection by pathogens, viruses or other agent; and other observable, measurable, or detectable physiological properties such as: biosynthesis of at least one metabolite, lipid, DNA, RNA or protein; chromosomal silencing, activation, heterochromatization, euchromoatinization or recombination; gene expression, gene silencing, gene splicing, gene recombination or gene-activation; RNA production, expression, transcription, processing splicing, transport, localization or modification; protein production, expression, secretion, folding, assembly, transport, localization, cell surface presentation, secretion or integration into a cell or organelle membrane; protein modification including but not limited to post-translational modification, processing, enzymatic modification, proteolysis, glycosylation, phosphorylation, dephosphorylation; cell division including mitosis, meiosis or fission or cell fusion; high level RNA or protein production or yield.

Physiological properties may be observed, detected or measured using routine assays known in the art, including but not limited to tests and methods described in reference guides and manuals such as the Current Protocols series. This series includes common protocols in various fields and is available through the Wiley Publishing House. The protocols in these reference guides are illustrative of the methods that can be used to observe, detect or measure physiological properties of cells. The skilled worker would readily recognize any one or more of these methods may be used to observe, detect or measure the physiological properties disclosed herein.

Many markers, dyes or reporters, including protein markers expressed as fusion proteins comprising an autofluorescent protein, that can be used to measure the level, activity or content of cellular compartments or organelles including but not limited to ribosomes, mitochondria, ER, rER, golgi, TGN, vesicles, endosomes and plasma membranes in cells are compatible with the testing of individual viable cells. In some embodiments fluorescence activated cell sorting or a cell sorter can be used. In some embodiments, cells or cell lines isolated or produced to comprise a CFTR can be tested using these markers, dyes or reporters at the same time, subsequent, or prior to isolation, testing or production of the cells or cell lines comprising a CFTR. In some embodiments, the level, activity or content of one or more of the cellular compartments or organelles can be correlated with improved, increased, native, non-cytotoxic, viable or optimal expression, function, activity, folding, assembly modification, post-translational modification, secretion, cell surface presentation, membrane integration, pharmacology, yield or physiology of a CFTR. In some embodiments, cells or cell lines comprising the level, activity or content of at least one cellular compartment or organelle that is correlated with improved, increased, native, non-cytotoxic, viable or optimal expression, function, activity, folding, assembly modification, post-translational modification, secretion, cell surface presentation, membrane integration, pharmacology, yield or physiology of a CFTR can be isolated. In some embodiments, cells or cell lines comprising the CFTR and the level, activity or content of at least one cellular compartment or organelle that is correlated with improved, increased, native, non-cytotoxic, viable or optimal expression, function, activity, folding, assembly modification, post-translational modification, secretion, cell surface presentation, membrane integration, pharmacology, yield or physiology of the CFTR can be isolated. In some embodiments the isolation of the cells is performed using cell sorting or fluorescence activated cell sorting.

The nucleic acid encoding the CFTR can be genomic DNA or cDNA. In some embodiments, the nucleic acid encoding the CFTR comprises one or more substitutions, mutations, or deletions, as compared to a wild type CFTR (SEQ ID NO: 1), that may or may not result in an amino acid substitution. In some embodiments, the nucleic acid is a fragment of the nucleic acid sequence provided. Such CFTR that are fragments or have such modifications retain at least one biological property of a CFTR, e.g., its ability to conduct chloride ions or be modulated by forskolin. The invention encompasses cells and cell lines stably expressing a CFTR-encoding nucleotide sequence that is at least about 85% identical to a sequence disclosed herein. In some embodiments, the CFTR-encoding sequence identity is at least 85%, 90%, 95%, 96%, 97%, 98%, 99% or higher compared to a CFTR sequence provided herein. The invention also encompasses cells and cell lines wherein a nucleic acid encoding a CFTR hybridizes under stringent conditions to a nucleic acid provided herein encoding the CFTR.

In some embodiments, the cell or cell line comprises a CFTR-encoding nucleic acid sequence comprising a substitution compared to a sequence provided herein by at least one but less than 10, 20, 30, or 40 nucleotides, up to or equal to 1%, 5%, 10% or 20% of the nucleotide sequence or a sequence substantially identical thereto (e.g., a sequence at least 85%, 90%, 95%, 96%, 97%, 98%, 99% or higher identical thereto, or that is capable of hybridizing under stringent conditions to the sequences disclosed). Such substitutions include single nucleotide polymorphisms (SNPs) and other allelic variations. In some embodiments, the cell or cell line comprises a CFTR-encoding nucleic acid sequence comprising an insertion into or deletion from the sequences provided herein by less than 10, 20, 30, or 40 nucleotides up to or equal to 1%, 5%, 10% or 20% of the nucleotide sequence or from a sequence substantially identical thereto.

In some embodiments, where the nucleic acid substitution or modification results in an amino acid change, such as an amino acid substitution, the native amino acid may be replaced by a conservative or non-conservative substitution (e.g., SEQ ID NO: 7). In some embodiments, the sequence identity between the original and modified polypeptide sequence can differ by about 1%, 5%, 10% or 20% of the polypeptide sequence or from a sequence substantially identical thereto (e.g., a sequence at least 85%, 90%, 95%, 96%, 97%, 98%, 99% or higher identical thereto). Those of skill in the art will understand that a conservative amino acid substitution is one in which the amino acid side chains are similar in structure and/or chemical properties and the substitution should not substantially change the structural characteristics of the parent sequence. In embodiments comprising a nucleic acid comprising a mutation, the mutation may be a random mutation or a site-specific mutation.

Conservative modifications will produce CFTRs having functional and chemical characteristics similar to those of the unmodified CFTR. A “conservative amino acid substitution” is one in which an amino acid residue is substituted by another amino acid residue having a side chain R group with similar chemical properties to the parent amino acid residue (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative amino acid substitution is any change having a positive value in the PAM250 log-likelihood matrix disclosed in Gonnet et al., Science 256:1443-45 (1992). A “moderately conservative” replacement is any change having a nonnegative value in the PAM250 log-likelihood matrix.

The invention encompasses cells or cell lines that comprise a mutant form of CFTR. More than 1,000 CFTR mutations have been identified, and the cells or cell lines of the invention may comprise any of these mutants of CFTRs. Such cells, cell lines, and collections of cell lines are useful to determine the activity of a mutant CFTR and the differential activity of a modulator on different mutant CFTRs.

The invention further comprises cells or cell lines that co-express other proteins with CFTR. Such other proteins may be integrated into the host cell's genome, or gene-activated, or induced. They may be expressed sequentially (before or after) with respect to CFTR or co-transfected with CFTR on the same or different vectors. In some embodiments, the co-expressed protein may be any of the following: genetic modifiers of CFTR (e.g., α1-antitrypsin, glutathione S-transferase, mannose binding lectin 2 (MBL2), nitric oxide synthase 1 (NOS1), glutamine-cysteine ligase gene (GCLC), FCgamma receptor II (FCγRII)); AMP activated protein kinase (AMPK), which phosphorylates and inhibits CFTR and may be important for airway inflammation and ischemia; transforming growth factor β1 (TGF-β1), which downregulates CFTR expression such that co-expression of TGFβ1 and CFTR may allow for identifying modulators of this interaction; tumor necrosis factor α (TNF-α), which downregulates CFTR expression such that coexpression TNF-α and CFTR may allow for identifying blockers of this interaction; β adrenergic receptor, which colocalizes with CFTR at the apical membrane and the stimulation of a subtype of β adrenergic receptor (β₂) increases CFTR activity; syntaxin 1a, which inhibits CFTR chloride channels by means of direct and domain-specific protein-protein interactions and may have therapeutic uses; synaptosome-associated protein 23, which physically associates with and inhibits CFTR; an epithelial sodium ion channel (ENaC), i.e., SCNN1A, SCNN1B or SCNN1G, to study binding interactions that stabilize CFTR at the cell surface; PDZK1 (PDZ domain containing 1) (also referred as CFTR-associated protein of 70 kDa (CAP70)), which potentiates CFTR chloride current; the endocytic complex AP2, which interacts with CFTR and facilitates efficient entry of CFTR into clathrin-coated vesicles; cyclic guanosine monophosphate (cGMP)-dependent protein kinase 2 (PRKG2), which is an upstream cGMP dependent kinase that phosphorylates and activates CFTR; protein kinase A and protein kinase C; protein phosphatase 2 (PP2A); guanine nucleotide binding protein (G protein), beta polypeptide 2-like 1 (RACK1); Rho family of GTPases; Rab GTPases, SNARE proteins; potassium channel proteins (e.g., ROMK1 and ROMK2); guanylyl cyclase c (GC-C or GUCY2C), which interacts with CFTR; chloride channel 2 (CLCN2 or CLC2), which is proposed to cause net Cl-efflux in gut such that coexpression of both CLCN2 and CFTR may allow for screens demonstrating maximal fluid efflux; solute carrier family 9 isoform A3 (NHE3-SLC9A3/sodium-hydrogen exchanger) or solute carrier family 26 isoform A3 (DRA-SLC26A3/sodium-hydrogen exchanger), to construct a rheostat biosensor for sodium intake/chloride efflux; cyclic nucleotide gated channel (CNGA2), which may be used as a HTS platform with a calcium readout; or a yellow fluorescent protein (YFP or variants thereof such as YFP H148Q/I152L) for usage in YFP halide quench assays.

In some embodiments, the CFTR-encoding nucleic acid sequence further comprises a tag. Such tags may encode, for example, a HIS tag, a myc tag, a hemagglutinin (HA) tag, protein C, VSV-G, FLU, yellow fluorescent protein (YFP), mutant YFP (meYFP), green fluorescent protein (GFP), FLAG, BCCP, maltose binding protein tag, Nus-tag, Softag-1, Softag-2, Strep-tag, S-tag, thioredoxin, GST, V5, TAP or CBP. A tag may be used as a marker to determine CFTR expression levels, intracellular localization, protein-protein interactions, CFTR regulation, or CFTR function. Tags may also be used to purify or fractionate CFTR. One example of a tag is meYFP-H1480/I152L (SEQ ID NO: 5).

Host cells used to produce a cell or cell line of the invention may express endogenous CFTR in its native state or lack expression of any CFTR. The host cell may be a primary, germ, or stem cell, including but not being limited to an embryonic stem cell. The host cell may also be an immortalized cell. Primary or immortalized host cells may be derived from mesoderm, ectoderm or endoderm layers of eukaryotic organisms. The host cell may include but not be limited to endothelial, epidermal, mesenchymal, neural, renal, hepatic, hematopoietic, or immune cells. For example, the host cells may include but not be limited to intestinal crypt or villi cells, clara cells, colon cells, intestinal cells, goblet cells, enterochromafin cells, enteroendocrine cells. The host cells may include but not be limited to be eukaryotic, prokaryotic, mammalian, human, primate, bovine, porcine, feline, rodent, marsupial, murine or other cells. The host cells may also be nonmammalian, including but not being limited to yeast, insect, fungus, plant, lower eukaryotes and prokaryotes. Such host cells may provide backgrounds that are more divergent for testing CFTR modulators with a greater likelihood for the absence of expression products provided by the cell that may interact with the target. In preferred embodiments, the host cell is a mammalian cell. Examples of host cells that may be used to produce a cell or cell line of the invention include but are not limited to: Chinese hamster ovary (CHO) cells, established neuronal cell lines, pheochromocytomas, neuroblastomas fibroblasts, rhabdomyosarcomas, dorsal root ganglion cells, NS0 cells, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), L-cells, HEK-293 (ATCC CRL1573) and PC12 (ATCC CRL-1721), HEK293T (ATCC CRL-11268), RBL (ATCC CRL-1378), SH-SY5Y (ATCC CRL-2266), MDCK (ATCC CCL-34), SJ-RH30 (ATCC CRL-2061), HepG2 (ATCC HB-8065), ND7/23 (ECACC 92090903), CHO (ECACC 85050302), Vero (ATCC CCL 81), Caco-2 (ATCC HTB 37), K562 (ATCC CCL 243), Jurkat (ATCC TIB-152), Per.C6 (Crucell, Leiden, The Netherlands), Huvec (ATCC Human Primary PCS 100-010, Mouse CRL 2514, CRL 2515, CRL 2516), HuH-7D12 (ECACC 01042712), 293 (ATCC CRL 10852), A549 (ATCC CCL 185), IMR-90 (ATCC CCL 186), MCF-7 (ATC HTB-22), U-2 OS (ATCC HTB-96), T84 (ATCC CCL 248), or any established cell line (polarized or nonpolarized) or any cell line available from repositories such as the American Type Culture Collection (ATCC, 10801 University Blvd. Manassas, Va. 20110-2209 USA) or European Collection of Cell Cultures (ECACC, Salisbury Wiltshire SP4 0JG England). Host cells used to produce a cell or cell line of the invention may be in suspension. For example, the host cells may be adherent cells adapted to suspension.

In certain embodiments, the methods described herein rely on the genetic variability and diversity in a population of cells, such as a cell line or a culture of immortalized cells. In particular, provided herein are cells, and methods for generating such cells, that express a CFTR endogenously, i.e., without the introduction of a nucleic acid encoding a CFTR. In certain embodiments, the isolated cell expressing the CFTR is represented by not more than 1 in 10, 1 in 100, 1 in 1000, 1 in 10,000, 1 in 100,000, 1 in 1,000,000 or 1 in 10,000,000 cells in a population of cells. The population of cells can be primary cells harvested from organisms. In certain embodiments, the population of cells is not known to express CFTR. In certain embodiments, genetic variability and diversity may also be increased using natural processes known to a person skilled in the art. Any suitable methods for creating or increasing genetic variability and/or diversity may be performed on host cells. In some cases, genetic variability may be due to modifications in regulatory regions of a gene encoding for CFTR. Cells expressing a particular CFTR can then be selected as described herein.

In other embodiments, genetic variability may be achieved by exposing a cell to UV light and/or x-rays (e.g., gamma-rays). In other embodiments, genetic variability may be achieved by exposing cells to EMS (ethyl methane sultonate). In some embodiments, genetic variability may be achieved by exposing cells to mutagens, carcinogens, or chemical agents. Non-limiting examples of such agents include deaminating agents such as nitrous acid, intercalating agents, and alkylating agents. Other non-limiting examples of such agents include bromine, sodium azide, and benzene. In specific embodiments, genetic variability may be achieved by exposing cells to growth conditions that are sub-optimal; e.g., low oxygen, low nutrients, oxidative stress or low nitrogen. In certain embodiments, enzymes that result in DNA damage or that decrease the fidelity of DNA replication or repair (e.g. mismatch repair) can be used to increase genetic variability. In certain embodiments, an inhibitor of an enzyme involved in DNA repair is used. In certain embodiments, a compound that reduces the fidelity of an enzyme involved in DNA replication is used. In certain embodiments, proteins that result in DNA damage and/or decrease the fidelity of DNA replication or repair are introduced into cells (co-expressed, injected, transfected, electroporated).

The duration of exposure to certain conditions or agents depend on the conditions or agents used. In some embodiments, seconds or minutes of exposure is sufficient. In other embodiments, exposure for a period of hours, days or months are necessary. The skilled artisan will be aware what duration and intensity of the condition can be used.

In some cases, a method that increases genetic variability may produce a mutation or alteration in a promoter region of a gene that leads to a change in the transcriptional regulation of the CFTR gene, e.g., gene activation, wherein the gene is more highly expressed than a gene with an unaltered promoter region. Generally, a promoter region includes a genomic DNA sequence upstream of a transcription start site that regulates gene transcription, and may include the minimal promoters and/or enhancers and/or repressor regions. A promoter region may range from about 20 basepairs (bps) to about 10,000 bps or more. In specific embodiments, a method that increases gene variability produces a mutation or alteration in an intron of a CFTR gene that leads to a change in the transcriptional regulation of the gene, e.g., gene activation wherein the gene is more highly expressed than gene with an unaltered intron. In certain embodiments, untranscribed genomic DNA is modified. For example, promoter, enhancer, modifier, or repressor regions can be added, deleted, or modified. In these cases, transcription of a CFTR transcript that is under control of the modified regulatory region can be used as a read-out. For example, if a repressor is deleted, the transcript of the CFTR gene that is repressed by the repressor is tested for increased transcription levels.

In certain embodiments, the genome of a cell or an organism can be mutated by site-specific mutagenesis or homologous recombination. In certain embodiments, oligonucleotide- or triplex-mediated recombination can be employed. See, e.g., Faruqi et al., 2000, Molecular and Cellular Biology 20:990-1000 and Schleifman et al., 2008, Methods Molecular Biology 435:175-90.

In certain embodiments, fluorogenic oligonucleotide probes or molecular beacons can be used to select cells in which the genetic modification has been successful, i.e., cells in which the transgene or the gene of interest is expressed. To identify cells in which a mutagenic or homologous recombination event has been successful, a fluorogenic oligonucleotide that specifically hybridizes to the mutagenized or recombined CFTR transcript can be used.

Once cells that endogenously express CFTR are isolated, these cells can be immortalized and cell lines generated. These cells or cell lines can be used with the assays and screening methods disclosed herein.

In one embodiment, the host cell is an embryonic stem cell that is then used as the basis for the generation of transgenic animals. Embryonic stem cells stably expressing CFTR, and preferably a functional introduced CFTR, may be implanted into organisms directly, or their nuclei may be transferred into other recipient cells and these may then be implanted, or they may be used to create transgenic animals.

As will be appreciated by those of skill in the art, any vector that is suitable for use with the host cell may be used to introduce a nucleic acid encoding CFTR into the host cell. Examples of vectors that may be used to introduce the CFTR encoding nucleic acids into host cells include but are not limited to plasmids, viruses, including retroviruses and lentiviruses, cosmids, artificial chromosomes and may include for example, pFN11A (BIND) Flexi®, pGL4.31, pFC14A (HaloTag® 7) CMV Flexi®, pFC14K (HaloTag® 7) CMV Flexi®, pFN24A (HaloTag® 7) CMVd3 Flexi®, pFN24K (HaloTag® 7) CMVd3 Flexi®, HaloTag™ pHT2, pACT, pAdVAntage™, pALTER®-MAX, pBIND, pCAT®3-Basic, pCAT®3-Control, pCAT®3-Enhancer, pCAT®3-Promoter, pCI, pCMVTNT™, pG5luc, pSI, pTARGET™, pTNT™, pF12A RM Flexi®, pF12K RM Flexi®, pReg neo, pYES2/GS, pAd/CMVN5-DEST Gateway® Vector, pAd/PL-DEST™ Gateway® Vector, Gateway® pDEST™27 Vector,Gateway® pEF-DEST51 Vector, Gateway® pcDNA™-DEST47 vector, pCMV/Bsd Vector, pEF6/His A, B, & C, pcDNA™6.2-DEST, pLenti6/TR, pLP-AcGFP1-C, pLPS-AcGFP1-N, pLP-IRESneo, pLP-TRE2, pLP-RevTRE, pLP-LNCX, pLP-CMV-HA, pLP-CMV-Myc, pLP-RetroQ, pLP-CMVneo, pCMV-Script, pcDNA3.1 Hygro, pcDNA3.1neo, pcDNA3.1puro, pSV2neo, pIRES puro, and pSV2 zeo. In some embodiments, the vectors comprise expression control sequences such as constitutive or conditional promoters. One of ordinary skill in the art will be able to select such sequences. For example, suitable promoters include but are not limited to CMV, TK, SV40, and EF-1α. In some embodiments, the promoters are inducible, temperature regulated, tissue specific, repressible, heat-shock, developmental, cell lineage specific, eukaryotic, prokaryotic or temporal promoters or a combination or recombination of unmodified or mutagenized, randomized, shuffled sequences of any one or more of the above. In other embodiments, CFTR is expressed by gene activation or when a gene encoding a CFTR is episomal. Nucleic acids encoding CFTRs may preferably be constitutively expressed.

In some embodiments, the vector encoding CFTR lacks a selectable marker or drug resistance gene. In other embodiments, the vector optionally comprises a nucleic acid encoding a selectable marker such as a protein that confers drug or antibiotic resistance. If more than one of the drug resistance markers are the same, simultaneous selection may be achieved by increasing the level of the drug. Suitable markers will be well-known to those of skill in the art and include but are not limited to genes conferring resistance to any one of the following: Neomycin/G418, Puromycin, hygromycin, Zeocin, methotrexate and blasticidin. Although drug selection (or selection using any other suitable selection marker) is not a required step, it may be used to enrich the transfected cell population for stably transfected cells, provided that the transfected constructs are designed to confer drug resistance. If subsequent selection of cells expressing CFTR is accomplished using signaling probes, selection too soon following transfection can result in some positive cells that may only be transiently and not stably transfected. However, this can be minimized by allowing sufficient cell passage allowing for dilution of transient expression in transfected cells.

In some embodiments, the vector comprises a nucleic acid sequence encoding an RNA tag sequence. “Tag sequence” refers to a nucleic acid sequence that is an expressed RNA or portion of an RNA that is to be detected by a signaling probe. Signaling probes may detect a variety of RNA sequences. Any of these RNAs may be used as tags. Signaling probes may be directed against the RNA tag by designing the probes to include a portion that is complementary to the sequence of the tag. The tag sequence may be a 3′ untranslated region of the plasmid that is cotranscribed and comprises a target sequence for signaling probe binding. The RNA encoding the gene of interest may include the tag sequence or the tag sequence may be located within a 5′-untranslated region or 3′-untranslated region. In some embodiments, the tag is not with the RNA encoding the gene of interest. The tag sequence can be in frame with the protein-coding portion of the message of the gene or out of frame with it, depending on whether one wishes to tag the protein produced. Thus, the tag sequence does not have to be translated for detection by the signaling probe. The tag sequences may comprise multiple target sequences that are the same or different, wherein one signaling probe hybridizes to each target sequence. The tag sequences may encode an RNA having secondary structure. The structure may be a three-arm junction structure. Examples of tag sequences that may be used in the invention, and to which signaling probes may be prepared, include but are not limited to the RNA transcript of epitope tags such as, for example, a HIS tag, a myc tag, a hemagglutinin (HA) tag, protein C, VSV-G, FLU, yellow fluorescent protein (YFP), green fluorescent protein, FLAG, BCCP, maltose binding protein tag, Nus-tag, Softag-1, Softag-2, Strep-tag, S-tag, thioredoxin, GST, V5, TAP or CBP. As described herein, one of ordinary skill in the art could create his or her own RNA tag sequences.

In another aspect of the invention, cells and cell lines of the invention have enhanced stability as compared to cells and cell lines produced by conventional methods. To identify stable expression, a cell or cell line's expression of CFTR is measured over a time course and the expression levels are compared. Stable cell lines will continue expressing CFTR throughout the time course. In some aspects of the invention, the time course may be for at least one week, two weeks, three weeks, etc., or at least one month, or at least two, three, four, five, six, seven, eight or nine months, or any length of time in between. Isolated cells and cell lines can be further characterized, such as by qRT-PCR and single end-point RT-PCR to determine the absolute amounts and relative amounts of CFTR being expressed. In some embodiments, stable expression is measured by comparing the results of functional assays over a time course. The measurement of stability based on functional assay provides the benefit of identifying clones that not only stably express the mRNA of the gene of interest, but also stably produce and properly process (e.g., post-translational modification, and localization within the cell) the protein encoded by the gene of interest that functions appropriately.

Cells and cell lines of the invention have the further advantageous property of providing assays with high reproducibility as evidenced by their Z′ factor. See Zhang J H, Chung T D, Oldenburg K R, “A Simple Statistical Parameter for Use in Evaluation and Validation of High Throughput Screening Assays.” J. Biomol. Screen. 1999; 4(2):67-73. Z′ values pertain to the quality of a cell or cell line because it reflects the degree to which a cell or cell line will respond consistently to modulators. Z′ is a statistical calculation that takes into account the signal-to-noise range and signal variability (i.e., from well to well) of the functional response to a reference compound across a multiwell plate. Z′ is calculated using data obtained from multiple wells with a positive control and multiple wells with a negative control. The ratio of their summated standard deviations multiplied by a factor of three to the difference in their mean values is subtracted from one to give the Z′ factor, according the equation below:

Z′ factor=1−((3σ_{positive control}+3σ_{negative control})/(μ_{positive control}−μ_{negative control}))

The theoretical maximum Z′ factor is 1.0, which would indicate an ideal assay with no variability and limitless dynamic range. As used herein, a “high Z′” refers to a Z′ factor of Z′ of at least 0.6, at least 0.7, at least 0.75 or at least 0.8, or any decimal in between 0.6 and 1.0. A score less than 0 is undesirable because it indicates that there is overlap between positive and negative controls. In the industry, for simple cell-based assays, Z′ scores up to 0.3 are considered marginal scores, Z′ scores between 0.3 and 0.5 are considered acceptable, and Z′ scores above 0.5 are considered excellent. Cell-free or biochemical assays may approach higher Z′ scores, but Z′ factor scores for cell-based systems tend to be lower because cell-based systems are complex.

As those of ordinary skill in the art will recognize, historically, cell-based assays using cells expressing even a single chain protein do not typically achieve a Z′ higher than 0.5 to 0.6. Cells and cell lines of the invention, on the other hand, have high Z′ values and advantageously produce consistent results in assays. CFTR expression cells and cell lines of the invention provided the basis for high-throughput screening (HTS) compatible assays because they generally have Z′ factor factors at least 0.82. In some aspects of the invention, the cells and cell lines result in Z′ of at least 0.3, at least 0.4, at least 0.5, at least 0.6, at least 0.7, or at least 0.8. In other aspects of the invention, the cells and cell lines of the invention result in a Z′ of at least 0.7, at least 0.75 or at least 0.8 maintained for multiple passages, e.g., between 5-20 passages, including any integer in between 5 and 20. In some aspects of the invention, the cells and cell lines result in a Z′ of at least 0.7, at least 0.75 or at least 0.8 maintained for 1, 2, 3, 4 or 5 weeks or 2, 3, 4, 5, 6, 7, 8 or 9 months, including any period of time in between.

Also according to the invention, cells and cell lines that express a form of a naturally occurring wild type CFTR or mutant CFTR can be characterized for chloride ion conductance. In some embodiments, the cells and cell lines of the invention express CFTR with “physiologically relevant” activity. As used herein, physiological relevance refers to a property of a cell or cell line expressing a CFTR whereby the CFTR conducts chloride ions as a naturally occurring CFTR of the same type and responds to modulators in the same ways that naturally occurring CFTR of the same type is modulated by the same modulators. CFTR-expressing cells and cell lines of this invention preferably demonstrate comparable function to cells that normally express native CFTR in a suitable assay, such as a membrane potential assay or a YFP halide quench assay using chloride or iodide as the ion conducted by CFTR, electrophysiology (e.g., patch clamp or Ussing), or by activation with forskolin. Such comparisons are used to determine a cell or cell line's physiological relevance.

In some embodiments, the cells and cell lines of the invention have increased sensitivity to modulators of CFTR. Cells and cell lines of the invention respond to modulators and conduct chloride ions with physiological range EC₅₀ or IC₅₀ values for CFTR. As used herein, EC₅₀ refers to the concentration of a compound or substance required to induce a half-maximal activating response in the cell or cell line. As used herein, IC₅₀ refers to the concentration of a compound or substance required to induce a half-maximal inhibitory response in the cell or cell line. EC₅₀ and IC₅₀ values may be determined using techniques that are well-known in the art, for example, a dose-response curve that correlates the concentration of a compound or substance to the response of the CFTR-expressing cell line. For example, the EC₅₀ for forskolin in a cell line of the invention is about 250 nM, and the EC₅₀ for forskolin in a stable CFTR-expressing fisher rat thyroid cell line disclosed in Galietta et al., Am J Physiol Cell Physiol. 281(5): C1734-1742 (2001) is between 250 nM and 500 nM.

A further advantageous property of the CFTR-expressing cells and cell lines of the inventions, flowing from the physiologically relevant function of the CFTR is that modulators identified in initial screening are functional in secondary functional assays, e.g., membrane potential assay, electrophysiology assay, YFP halide quench assay, radioactive iodine flux assay, rabbit intestinal-loop fluid secretion measurement assay, animal fecal output testing and measuring assay, or Ussing chamber assays. As those of ordinary skill in the art will recognize, compounds identified in initial screening assays typically must be modified, such as by combinatorial chemistry, medicinal chemistry or synthetic chemistry, for their derivatives or analogs to be functional in secondary functional assays. However, due to the high physiological relevance of the present CFTR cells and cell lines, many compounds identified therewith are functional without “coarse” tuning.

In some embodiments, properties of the cells and cell lines of the invention, such as stability, physiological relevance, reproducibility in an assay (Z′), or physiological EC₅₀ or IC₅₀ values, are achievable under specific culture conditions. In some embodiments, the culture conditions are standardized and rigorously maintained without variation, for example, by automation. Culture conditions may include any suitable conditions under which the cells or cell lines are grown and may include those known in the art. A variety of culture conditions may result in advantageous biological properties for any of the bitter receptors, or their mutants or allelic variants.

In other embodiments, the cells and cell lines of the invention with desired properties, such as stability, physiological relevance, reproducibility in an assay (Z′), or physiological EC₅₀ or IC₅₀ values, can be obtained within one month or less. For example, the cells or cell lines may be obtained within 2, 3, 4, 5, or 6 days, or within 1, 2, 3 or 4 weeks, or any length of time in between.

One aspect of the invention provides a collection or panel of cells and cell lines, each expressing a different form of CFTR (e.g., wild type, allelic variants, mutants, fragment, spliced variants etc.). The collection may include, for example, cells or cell lines expressing CFTR, CFTR ΔF508 and various other known mutant CFTRs. In some embodiment, the collections or panels include cells expressing other ion channel proteins. The collections or panels may additional comprise cells expressing control proteins. The collections or panels of the invention can be used for compound screening or profiling, e.g., to identify modulators that are active on some or all.

When collections or panels of cells or cell lines are produced, e.g., for drug screening, the cells or cell lines in the collection or panel may be derived from the same host cells and may further be matched such that they are the same (including substantially the same) with regard to one or more selective physiological properties. The “same physiological property” in this context means that the selected physiological property is similar enough amongst the members in the collection or panel such that the cell collection or panel can produce reliable results in drug screening assays; for example, variations in readouts in a drug screening assay will be due to, e.g., the different biological activities of test compounds on cells expressing different forms of CFTR, rather than due to inherent variations in the cells. For example, the cells or cell lines may be matched to have the same growth rate, i.e., growth rates with no more than one, two, three, four, or five hours difference amongst the members of the cell collection or panel. This may be achieved by, for example, binning cells by their growth rate into five, six, seven, eight, nine, or ten groups, and creating a panel using cells from the same binned group. Methods of determining cell growth rate are well known in the art. The cells or cell lines in a panel also can be matched to have the same Z′ factor (e.g., Z′ factors that do not differ by more than 0.1), CFTR expression level (e.g., CFTR expression levels that do not differ by more than 5%, 10%, 15%, 20%, 25%, or 30%), adherence to tissue culture surfaces, and the like. Matched cells and cell lines can be grown under identical conditions, achieved by, e.g., automated parallel processing, to maintain the selected physiological property.

Matched cell panels of the invention can be used to, for example, identify modulators with defined activity (e.g., agonist or antagonist) on CFTR; to profile compound activity across different forms of CFTR; to identify modulators active on just one form of CFTR; and to identify modulators active on just a subset of CFTRs. The matched cell panels of the invention allow high throughput screening. Screenings that used to take months to accomplish can now be accomplished within weeks.

To make cells and cell lines of the invention, one can use, for example, the technology described in U.S. Pat. No. 6,692,965 and International Patent Publication WO/2005/079462. Both of these documents are incorporated herein by reference in their entirety for all purposes. This technology provides real-time assessment of millions of cells such that any desired number of clones (from hundreds to thousands of clones) may be selected. Using cell sorting techniques, such as flow cytometric cell sorting (e.g., with a FACS machine), magnetic cell sorting (e.g., with a MACS machine), or other fluorescence plate readers, including those that are compatible with high-throughput screening, one cell per well may be automatically deposited with high statistical confidence in a culture vessel (such as a 96 well culture plate). The speed and automation of the technology allows multigene cell lines to be readily isolated.

In some embodiments, the invention provides a panel of cell lines comprising at least 3, 5, 10, 25, 50, 100, 250, 500, 750, or 1000 cells or cell lines, each expressing a different CFTR mutant selected from the CFTR mutants set forth in Table 1 or Table 2. In certain embodiments, such a panel comprises at least 3, 5, 10, 25, 50, or 75 cells or cell lines, each expressing a different CFTR mutant selected from the CFTR mutants set forth in Table 2. For example, the panel may comprise a CFTR-ΔF508 expressing cell line. In certain embodiments, the panel comprises at least 3, 5, 10, 25, 50, or 100 cells or cell lines, each expressing a different CFTR mutant, wherein each CFTR mutant is a missense, nonsense, frameshift or RNA splicing mutation. In certain embodiments, such a panel comprises at least 3, 5, 10, 25, 50, or 100 cells or cell lines, each expressing a different CFTR mutant, wherein each CFTR mutant is associated with cystic fibrosis. In certain embodiments, such a panel comprises at least 3, 5, 10, 25, 50, or 100 cells or cell lines each expressing a different CFTR mutant, wherein each CFTR mutant is associated with congenital bilateral absence of the vas deferens. Such panels can be used for parallel high-throughput screening and cross-comparative characterization of small molecules with efficacy against the various isoforms of the CFTR protein. In certain embodiments, such a panel also comprises one or more cells or cell lines engineered or selected to express a protein of interest other than CFTR or CFTR mutant.

Using the technology, the RNA sequence for CFTR may be detected using a signaling probe, also referred to as a molecular beacon or fluorogenic probe. As described in, e.g., U.S. Pat. No. 6,692,965, a molecular beacon typically is a nucleic acid probe that recognizes and reports the presence of a specific nucleic acid sequence. The probes may be hairpin-shaped sequences with a central stretch of nucleotides complementary to the target sequence, and termini comprising short mutually complementary sequences. One terminus is covalently bound to a fluorophore and the other to a quenching moiety. When in their native state with hybridized termini, the proximity of the fluorophore and the quencher is such that no fluorescence is produced. The beacon undergoes a spontaneous fluorogenic conformational change when hybridized to its target nucleic acid. In some embodiments, the molecular beacon (or fluorogenic probe) recognizes a target tag sequence as described above. In another embodiment, the molecular beacon (or fluorogenic probe) recognizes a sequence within CFTR itself. Signaling probes may be directed against the RNA tag or CFTR sequence by designing the probes to include a portion that is complementary to the RNA sequence of the tag or the CFTR, respectively.

Nucleic acids comprising a sequence encoding a CFTR, or the sequence of a CFTR and a tag sequence, and optionally a nucleic acid encoding a selectable marker may be introduced into selected host cells by well known methods. The methods include but are not limited to transfection, viral delivery, protein or peptide mediated insertion, coprecipitation methods, lipid based delivery reagents (lipofection), cytofection, lipopolyamine delivery, dendrimer delivery reagents, electroporation or mechanical delivery. Examples of transfection reagents are GENEPORTER, GENEPORTER2, LIPOFECTAMINE™, LIPOFECTAMINE™ 2000, FUGENE® 6, FUGENE® HD, TFX™-10, TFX™-20, TFX™-50, OLIGOFECTAMINE, TRANSFAST, TRANSFECTAM, GENESHUTTLE, TROJENE, GENESILENCER, X-TREMEGENE, PERFECTIN, CYTOFECTIN, SIPORT, UNIFECTOR, SIFECTOR, TRANSIT-LT1, TRANSIT-LT2, TRANSIT-EXPRESS, IFECT, RNAI SHUTTLE, METAFECTENE, LYOVEC, LIPOTAXI, GENEERASER, GENEJUICE, CYTOPURE, JETSI, JETPEI, MEGAFECTIN, POLYFECT, TRANSMESSANGER, RNAiFECT, SUPERFECT, EFFECTENE, TF-PEI-KIT, CLONFECTIN, AND METAFECTINE.

Following introduction of the CFTR coding sequences or the CFTR activation sequences into host cells and optional subsequent drug selection, molecular beacons (e.g., fluorogenic probes) are introduced into the cells and cell sorting is used to isolate cells positive for their signals. Multiple rounds of sorting may be carried out, if desired. In one embodiment, the flow cytometric cell sorter is a FACS machine. MACS (magnetic cell sorting) or laser ablation of negative cells using laser-enabled analysis and processing can also be used. Other fluorescence plate readers, including those that are compatible with high-throughput screening can also be used. According to this method, cells expressing CFTR are detected and recovered. The CFTR sequence may be integrated at different locations of the genome in the cell. The expression level of the introduced genes encoding the CFTR may vary based upon integration site. The skilled worker will recognize that sorting can be gated for any desired expression level. Further, stable cell lines may be obtained wherein one or more of the introduced genes encoding a CFTR is episomal or results from gene activation.

Signaling probes useful in this invention are known in the art and generally are oligonucleotides comprising a sequence complementary to a target sequence and a signal emitting system so arranged that no signal is emitted when the probe is not bound to the target sequence and a signal is emitted when the probe binds to the target sequence. By way of non-limiting illustration, the signaling probe may comprise a fluorophore and a quencher positioned in the probe so that the quencher and fluorophore are brought together in the unbound probe. Upon binding between the probe and the target sequence, the quencher and fluorophore separate, resulting in emission of signal. International publication WO/2005/079462, for example, describes a number of signaling probes that may be used in the production of the cells and cell lines of this invention.

Nucleic acids encoding signaling probes may be introduced into the selected host cell by any of numerous means that will be well-known to those of skill in the art, including but not limited to transfection, coprecipitation methods, lipid based delivery reagents (lipofection), cytofection, lipopolyamine delivery, dendrimer delivery reagents, electroporation or mechanical delivery. Examples of transfection reagents are GENEPORTER, GENEPORTER2, LIPOFECTAMINE, LIPOFECTAMINE 2000, FUGENE 6, FUGENE HD, TFX-10, TFX-20, TFX-50, OLIGOFECTAMINE, TRANSFAST, TRANSFECTAM, GENESHUTTLE, TROJENE, GENESILENCER, X-TREMEGENE, PERFECTIN, CYTOFECTIN, SIPORT, UNIFECTOR, SIFECTOR, TRANSIT-LT1, TRANSIT-LT2, TRANSIT-EXPRESS, IFECT, RNAI SHUTTLE, METAFECTENE, LYOVEC, LIPOTAXI, GENEERASER, GENEJUICE, CYTOPURE, JETSI, JETPEI, MEGAFECTIN, POLYFECT, TRANSMESSANGER, RNAiFECT, SUPERFECT, EFFECTENE, TF-PEI-KIT, CLONFECTIN, AND METAFECTINE.

In one embodiment, the signaling probes are designed to be complementary to either a portion of the RNA encoding a CFTR or to portions of their 5′ or 3′ untranslated regions. Even if the signaling probe designed to recognize a messenger RNA of interest is able to detect spuriously endogenously existing target sequences, the proportion of these in comparison to the proportion of the sequence of interest produced by transfected cells is such that the sorter is able to discriminate the two cell types.

The expression level of CFTR may vary from cell or cell line to cell or cell line. The expression level in a cell or cell line also may decrease over time due to epigenetic events such as DNA methylation and gene silencing and loss of transgene copies. These variations can be attributed to a variety of factors, for example, the copy number of the transgene taken up by the cell, the site of genomic integration of the transgene, and the integrity of the transgene following genomic integration. One may use FACS or other cell sorting methods (i.e., MACS) to evaluate expression levels. Additional rounds of introducing signaling probes may be used, for example, to determine if and to what extent the cells remain positive over time for any one or more of the RNAs for which they were originally isolated.

In another embodiment of the invention, adherent cells can be adapted to suspension before or after cell sorting and isolating single cells. In other embodiments, isolated cells may be grown individually or pooled to give rise to populations of cells. Individual or multiple cell lines may also be grown separately or pooled. If a pool of cell lines is producing a desired activity or has a desired property, it can be further fractionated until the cell line or set of cell lines having this effect is identified. Pooling cells or cell lines may make it easier to maintain large numbers of cell lines without the requirements for maintaining each separately. Thus, a pool of cells or cell lines may be enriched for positive cells. An enriched pool may have at least 50%, at least 60%, at least 70%, at least 80%, at least 90% or 100% are positive for the desired property or activity.

In a further aspect, the invention provides a method for producing the cells and cell lines of the invention. In one embodiment, the method comprises the steps of:

a) providing a plurality of cells that express mRNA encoding CFTR;

b) dispersing cells individually into individual culture vessels, thereby providing a plurality of separate cell cultures

c) culturing the cells under a set of desired culture conditions using automated cell culture methods characterized in that the conditions are substantially identical for each of the separate cell cultures, during which culturing the number of cells in each separate cell culture is normalized, and wherein the separate cultures are passaged on the same schedule;

d) assaying the separate cell cultures for at least one desired characteristic of CFTR at least twice; and

e) identifying a separate cell culture that has the desired characteristic in both assays.

According to the method, the cells are cultured under a desired set of culture conditions. The conditions can be any desired conditions. Those of skill in the art will understand what parameters are comprised within a set of culture conditions. For example, culture conditions include but are not limited to: the media (Base media (DMEM, MEM, RPMI, serum-free, with serum, fully chemically defined, without animal-derived components), mono and divalent ion (sodium, potassium, calcium, magnesium) concentration, additional components added (amino acids, antibiotics, glutamine, glucose or other carbon source, HEPES, channel blockers, modulators of other targets, vitamins, trace elements, heavy metals, co-factors, growth factors, anti-apoptosis reagents), fresh or conditioned media, with HEPES, pH, depleted of certain nutrients or limiting (amino acid, carbon source)), level of confluency at which cells are allowed to attain before split/passage, feeder layers of cells, or gamma-irradiated cells, CO₂, a three gas system (oxygen, nitrogen, carbon dioxide), humidity, temperature, still or on a shaker, and the like, which will be well known to those of skill in the art.

The cell culture conditions may be chosen for convenience or for a particular desired use of the cells. Advantageously, the invention provides cells and cell lines that are optimally suited for a particular desired use. That is, in embodiments of the invention in which cells are cultured under conditions for a particular desired use, cells are selected that have desired characteristics under the condition for the desired use.

By way of illustration, if cells will be used in assays in plates where it is desired that the cells are adherent, cells that display adherence under the conditions of the assay may be selected. Similarly, if the cells will be used for protein production, cells may be cultured under conditions appropriate for protein production and selected for advantageous properties for this use.

In some embodiments, the method comprises the additional step of measuring the growth rates of the separate cell cultures. Growth rates may be determined using any of a variety of techniques means that will be well known to the skilled worker. Such techniques include but are not limited to measuring ATP, cell confluency, light scattering, optical density (e.g., OD 260 for DNA). Preferably growth rates are determined using means that minimize the amount of time that the cultures spend outside the selected culture conditions.

In some embodiments, cell confluency is measured and growth rates are calculated from the confluency values. In some embodiments, cells are dispersed and clumps removed prior to measuring cell confluency for improved accuracy. Means for monodispersing cells are well-known and can be achieved, for example, by addition of a dispersing reagent to a culture to be measured. Dispersing agents are well-known and readily available, and include but are not limited to enzymatic dispersing agents, such as trypsin, and EDTA-based dispersing agents. Growth rates can be calculated from confluency date using commercially available software for that purpose such as HAMILTON VECTOR. Automated confluency measurement, such as using an automated microscopic plate reader is particularly useful. Plate readers that measure confluency are commercially available and include but are not limited to the CLONE SELECT IMAGER (Genetix). Typically, at least 2 measurements of cell confluency are made before calculating a growth rate. The number of confluency values used to determine growth rate can be any number that is convenient or suitable for the culture. For example, confluency can be measured multiple times over e.g., a week, 2 weeks, 3 weeks or any length of time and at any frequency desired.

When the growth rates are known, according to the method, the plurality of separate cell cultures are divided into groups by similarity of growth rates. By grouping cultures into growth rate bins, one can manipulate the cultures in the group together, thereby providing another level of standardization that reduces variation between cultures. For example, the cultures in a bin can be passaged at the same time, treated with a desired reagent at the same time, etc. Further, functional assay results are typically dependent on cell density in an assay well. A true comparison of individual clones is only accomplished by having them plated and assayed at the same density. Grouping into specific growth rate cohorts enables the plating of clones at a specific density that allows them to be functionally characterized in a high throughput format.

The range of growth rates in each group can be any convenient range. It is particularly advantageous to select a range of growth rates that permits the cells to be passaged at the same time and avoid frequent renormalization of cell numbers. Growth rate groups can include a very narrow range for a tight grouping, for example, average doubling times within an hour of each other. But according to the method, the range can be up to 2 hours, up to 3 hours, up to 4 hours, up to 5 hours or up to 10 hours of each other or even broader ranges. The need for renormalization arises when the growth rates in a bin are not the same so that the number of cells in some cultures increases faster than others. To maintain substantially identical conditions for all cultures in a bin, it is necessary to periodically remove cells to renormalize the numbers across the bin. The more disparate the growth rates, the more frequently renormalization is needed.

In step d) the cells and cell lines may be tested for and selected for any physiological property including but not limited to: a change in a cellular process encoded by the genome; a change in a cellular process regulated by the genome; a change in a pattern of chromosomal activity; a change in a pattern of chromosomal silencing; a change in a pattern of gene silencing; a change in a pattern or in the efficiency of gene activation; a change in a pattern or in the efficiency of gene expression; a change in a pattern or in the efficiency of RNA expression; a change in a pattern or in the efficiency of RNAi expression; a change in a pattern or in the efficiency of RNA processing; a change in a pattern or in the efficiency of RNA transport; a change in a pattern or in the efficiency of protein translation; a change in a pattern or in the efficiency of protein folding; a change in a pattern or in the efficiency of protein assembly; a change in a pattern or in the efficiency of protein modification; a change in a pattern or in the efficiency of protein transport; a change in a pattern or in the efficiency of transporting a membrane protein to a cell surface change in growth rate; a change in cell size; a change in cell shape; a change in cell morphology; a change in % RNA,content; a change in % protein content; a change in % water content; a change in % lipid content; a change in ribosome content; a change in mitochondrial content; a change in ER mass; a change in plasma membrane surface area; a change in cell volume; a change in lipid composition of plasma membrane; a change in lipid composition of nuclear envelope; a change in protein composition of plasma membrane; a change in protein; composition of nuclear envelope; a change in number of secretory vesicles; a change in number of lysosomes; a change in number of vacuoles; a change in the capacity or potential of a cell for: protein production, protein secretion, protein folding, protein assembly, protein modification, enzymatic modification of protein, protein glycosylation, protein phosphorylation, protein dephosphorylation, metabolite biosynthesis, lipid biosynthesis, DNA synthesis, RNA synthesis, protein synthesis, nutrient absorption, cell growth, mitosis, meiosis, cell division, to dedifferentiate, to transform into a stem cell, to transform into a pluripotent cell, to transform into a omnipotent cell, to transform into a stem cell type of any organ (i.e., liver, lung, skin, muscle, pancreas, brain, testis, ovary, blood, immune system, nervous system, bone, cardiovascular system, central nervous system, gastro-intestinal tract, stomach, thyroid, tongue, gall bladder, kidney, nose, eye, nail, hair, taste bud), to transform into a differentiated any cell type (i.e., muscle, heart muscle, neuron, skin, pancreatic, blood, immune, red blood cell, white blood cell, killer T-cell, enteroendocrine cell, taste, secretory cell, kidney, epithelial cell, endothelial cell, also including any of the animal or human cell types already listed that can be used for introduction of nucleic acid sequences), to uptake DNA, to uptake small molecules, to uptake fluorogenic probes, to uptake RNA, to adhere to solid surface, to adapt to serum-free conditions, to adapt to serum-free suspension conditions, to adapt to scaled-up cell culture, for use for large scale cell culture, for use in drug discovery, for use in high throughput screening, for use in a functional cell based assay, for use in membrane potential assays, for use in reporter cell based assays, for use in ELISA studies, for use in in vitro assays, for use in vivo applications, for use in secondary testing, for use in compound testing, for use in a binding assay, for use in panning assay, for use in an antibody panning assay, for use in imaging assays, for use in microscopic imaging assays, for use in multiwell plates, for adaptation to automated cell culture, for adaptation to miniaturized automated cell culture, for adaptation to large-scale automated cell culture, for adaptation to cell culture in multiwell plates (6, 12, 24, 48, 96, 384, 1536 or higher density), for use in cell chips, for use on slides, for use on glass slides, for microarray on slides or glass slides, for immunofluorescence studies, for use in protein purification, and for use in biologics production. Those of skill in the art will readily recognize suitable tests for any of the above-listed properties.

Tests that may be used to characterize cells and cell lines of the invention and/or matched panels of the invention include but are not limited to: amino acid analysis, DNA sequencing, protein sequencing, NMR, a test for protein transport, a test for nucleocytoplasmic transport, a test for subcellular localization of proteins, a test for subcellular localization of nucleic acids, microscopic analysis, submicroscopic analysis, fluorescence microscopy, electron microscopy, confocal microscopy, laser ablation technology, cell counting and Dialysis. The skilled worker would understand how to use any of the above-listed tests.

According to the method, cells may be cultured in any cell culture format so long as the cells or cell lines are dispersed in individual cultures prior to the step of measuring growth rates. For example, for convenience, cells may be initially pooled for culture under the desired conditions and then individual cells separated one cell per well or vessel. Cells may be cultured in multi-well tissue culture plates with any convenient number of wells. Such plates are readily commercially available and will be well knows to a person of skill in the art. In some cases, cells may preferably be cultured in vials or in any other convenient format, the various formats will be known to the skilled worker and are readily commercially available.

In embodiments comprising the step of measuring growth rate, prior to measuring growth rates, the cells are cultured for a sufficient length of time for them to acclimate to the culture conditions. As will be appreciated by the skilled worker, the length of time will vary depending on a number of factors such as the cell type, the chosen conditions, the culture format and may be any amount of time from one day to a few days, a week or more.

Preferably, each individual culture in the plurality of separate cell cultures is maintained under substantially identical conditions a discussed below, including a standardized maintenance schedule. Another advantageous feature of the method is that large numbers of individual cultures can be maintained simultaneously, so that a cell with a desired set of traits may be identified even if extremely rare. For those and other reasons, according to the invention, the plurality of separate cell cultures are cultured using automated cell culture methods so that the conditions are substantially identical for each well. Automated cell culture prevents the unavoidable variability inherent to manual cell culture.

Any automated cell culture system may be used in the method of the invention. A number of automated cell culture systems are commercially available and will be well-known to the skilled worker. In some embodiments, the automated system is a robotic system. Preferably, the system includes independently moving channels, a multichannel head (e.g., a 96-tip head) and a gripper or cherry-picking arm and a HEPA filtration device to maintain sterility during the procedure. The number of channels in the pipettor should be suitable for the format of the culture. Convenient pipettors have, e.g., 96 or 384 channels. Such systems are known and are commercially available. For example, a MICROLAB STAR™ instrument (Hamilton) may be used in the method of the invention. The automated system should be able to perform a variety of desired cell culture tasks. Such tasks will be known by a person of skill in the art. They include but are not limited to: removing media, replacing media, adding reagents, cell washing, removing wash solution, adding a dispersing agent, removing cells from a culture vessel, adding cells to a culture vessel an the like.

The production of a cell or cell line of the invention may include any number of separate cell cultures. However, the advantages provided by the method increase as the number of cells increases. There is no theoretical upper limit to the number of cells or separate cell cultures that can be utilized in the method. According to the invention, the number of separate cell cultures can be two or more but more advantageously is at least 3, 4, 5, 6, 7, 8, 9, 10 or more separate cell cultures, for example, at least 12, at least 15, at least 20, at least 24, at least 25, at least 30, at least 35, at least 40, at least 45, at least 48, at least 50, at least 75, at least 96, at least 100, at least 200, at least 300, at least 384, at least 400, at least 500, at least 1000, at least 10,000, at least 100,000, at least 500,000 or more.

A further advantageous property of the CFTR cells and cell lines of the invention is that they stably express CFTR in the absence of selective pressure. Selection pressure is applied in cell culture to select cells with desired sequences or traits, and is usually achieved by linking the expression of a polypeptide of interest with the expression of a selection marker that imparts to the cells resistance to a corresponding selective agent or pressure. Antibiotic selection includes, without limitation, the use of antibiotics (e.g., puromycin, neomycin, G418, hygromycin, bleomycin and the like). Non-antibiotic selection includes, without limitation, the use of nutrient deprivation, exposure to selective temperatures, exposure to mutagenic conditions and expression of fluorescent markers where the selection marker may be e.g., glutamine synthetase, dihydrofolate reductase (DHFR), oabain, thymidine kinase (TK), hypoxanthine guanine phosphororibosyltransferase (HGPRT) or a fluorescent protein such as GFP. Thus, in some embodiments, cells and cell lines of the invention are maintained in culture without any selective pressure. In further embodiments, cells and cell lines are maintained without any antibiotics. As used herein, cell maintenance refers to culturing cells after they have been selected as described above for their CFTR expression. Maintenance does not refer to the optional step of growing cells in a selective drug (e.g., an antibiotic) prior to cell sorting where drug resistance marker(s) introduced into the cells allow enrichment of stable transfectants in a mixed population.

Drug-free cell maintenance provides a number of advantages. For examples, drug-resistant cells do not always express the co-transfected transgene of interest at adequate levels, because the selection relies on survival of the cells that have taken up the drug resistant gene, with or without the transgene. Further, selective drugs are often mutagenic or otherwise interfere with the physiology of the cells, leading to skewed results in cell-based assays. For example, selective drugs may decrease susceptibility to apoptosis (Robinson et al., Biochemistry, 36(37):11169-11178 (1997)), increase DNA repair and drug metabolism (Deffie et al., Cancer Res. 48(13):3595-3602 (1988)), increase cellular pH (Thiebaut et al., J Histochem Cytochem. 38(5):685-690 (1990); Roepe et al., Biochemistry. 32(41):11042-11056 (1993); Simon et al., Proc Natl Acad Sci USA. 91(3):1128-1132 (1994)), decrease lysosomal and endosomal pH (Schindler et al., Biochemistry. 35(9):2811-2817 (1996); Altan et al., J Exp Med. 187(10):1583-1598 (1998)), decrease plasma membrane potential (Roepe et al., Biochemistry. 32(41):11042-11056 (1993)), increase plasma membrane conductance to chloride (Gill et al., Cell. 71(1):23-32 (1992)) and ATP (Abraham et al., Proc Natl Acad Sci USA. 90(1):312-316 (1993)), and increase rates of vesicle transport (Altan et al., Proc Natl Acad Sci USA. 96(8):4432-4437 (1999)). GFP, a commonly used non-antibiotic selective marker, may cause cell death in certain cell lines (Hanazono et al., Hum Gene Ther. 8(11):1313-1319 (1997)). Thus, the cells and cell lines of this invention allow screening assays that are free from any artifact caused by selective drugs or markers. In some preferred embodiments, the cells and cell lines of this invention are not cultured with selective drugs such as antibiotics before or after cell sorting, so that cells and cell lines with desired properties are isolated by sorting, even when not beginning with an enriched cell population.

In another aspect, the invention provides methods of using the cells and cell lines of the invention. The cells and cell lines of the invention may be used in any application for which functional CFTR or mutant CFTRs are needed. The cells and cell lines may be used, for example, but not limited to, in an in vitro cell-based assay or an in vivo assay where the cells are implanted in an animal (e.g., a non-human mammal) to, e.g., screen for CFTR (e.g., CFTR mutant) modulators; produce protein for crystallography and binding studies; and investigate compound selectivity and dosing, receptor/compound binding kinetic and stability, and effects of receptor expression on cellular physiology (e.g., electrophysiology, protein trafficking, protein folding, and protein regulation). The cells and cell lines of the invention also can be used in knock down studies to study the roles of mutant CFTRs.

Cells and cell lines expressing different forms of CFTR can be used separately or together to identify CFTR modulators, including those specific for a particular mutant CFTR and to obtain information about the activities of individual mutant CFTRs. The present cells and cell lines may be used to identify the roles of different forms of CFTR in different CFTR pathologies by correlating the identity of in vivo forms of mutant CFTR with the identify of known forms of CFTR based on their response to various modulators. This allows selection of disease- or tissue-specific CFTR modulators for highly targeted treatment of such CFTR-related pathologies.

Modulators include any substance or compound that alters an activity of a CFTR. Modulators help identifying the relevant mutant CFTRs implicated in CFTR pathologies (i.e., pathologies related to ion conductance through various CFTR channels), and selecting tissue specific compounds for the selective treatment of such pathologies or for the development of related compounds useful in those treatments. In other aspects, a modulator may change the ability of another modulator to affect the function of a CFTR. For example, a modulator of a mutant CFTR that is not activated by forskolin may render that form of CFTR susceptible to activation by forskolin.

Stable cell lines expressing a CFTR mutant and panels of such cell lines (see above) can be used to screen modulators (including agonists, antagonists, potentiators and inverse agonists), e.g., in high-throughput compatible assays. Modulators so identified can then be assayed against other CFTR alleles to identify specific modulators specific for given CFTR mutants.

In some embodiments, the present invention provides a method for generating an in-vitro-correlate (“IVC”) for an in vivo physiological property of interest. An IVC is generated by establishing the activity profile of a compound with an in vivo physiological property on different CFTR mutants, e.g., a profile of the effect of a compound on the physiological property of different CFTR mutants. This can be accomplished by using a panel of cells or cell lines as disclosed above. This activity profile is representative of the in vivo physiological property and thus is an IVC of a fingerprint for the physiological property. In some embodiments, the in-vitro-correlate is an in-vitro-correlate for a negative side effect of a drug. In other embodiments, the in-vitro-correlate is an in-vitro-correlate for a beneficial effect of a drug.

In some embodiments, the IVC can be used to predict or confirm one or more physiological properties of a compound of interest. The compound may be tested for its activity against different CFTR mutants and the resulting activity profile is compared to the activity profile of IVCs that are generated as described herein. The physiological property of the IVC with an activity profile most similar to the activity profile of a compound of interest is predicted to be and/or confirmed to be a physiological property of the compound of interest.

In some embodiments, an IVC is established by assaying the activities of a compound against different CFTR mutants, or combinations thereof. Similarly, to predict or confirm the physiological activity of a compound, the activities of the compound can be tested against different CFTR mutants.

In some embodiments, the methods of the invention can be used to determine and/or predict and/or confirm to what degree a particular physiological effect is caused by a compound of interest. In certain embodiments, the methods of the invention can be used to determine and/or predict and/or confirm the tissue specificity of a physiological effect of a compound of interest.

In more specific embodiments, the activity profile of a compound of interest is established by testing the activity of the compound in a plurality of in vitro assays using cell lines that are engineered to express different CFTR mutants (e.g., a panel of cells expressing different CFTR mutants). In some embodiments, testing of candidate drugs against a panel of CFTR mutants can be used to correlate specific targets to adverse or undesired side-effects or therapeutic efficacy observed in the clinic. This information may be used to select well defined targets in high-throughput screening or during development of drugs with maximal desired and minimal off-target activity.

In certain embodiments, the physiological parameter is measured using functional magnetic resonance imaging (“fMRI”). Other imaging methods can also be used, for example, computed tomography (CT); computed axial tomography (CAT) scanning; diffuse optical imaging (DOI); diffuse optical tomography (DOT); event-related optical signal (EROS); near infrared spectroscopy (NIRS); magnetic resonance imaging (MRI); magnetoencephalography (MEG); positron emission tomography (PET) and single photon emission computed tomography (SPECT).

In certain embodiments, if the IVC represents an effect of the compound on the central nervous system (“CNS”), an IVC may be established that correlates with an fMRI pattern in the CNS. IVCs may be generated that correlate with activity of compounds in various tests and models, including human and animal testing models. Human diseases and disorders are listed, e.g., in The Merck Manual, 18th Edition (Hardcover), Mark H. Beers (Author), Robert S. Porter (Editor), Thomas V. Jones (Editor). Mental diseases and disorders are listed, in e.g., Diagnostic and Statistical Manual of Mental Disorders (DSM-IV-TR) Fourth Edition (Text Revision), by American Psychiatric Association.

IVCs using CFTR can also be generated for the following properties: regulation, secretion, quality, clearance, production, viscosity, or thickness of mucous, water absorption, retention, balance, passing, or transport across epithelial tissues (especially of lung, kidney, vascular tissues, eye, gut, small intestine, large intestine); sensory or taste perception of compounds; neuronal firing or CNS activity in response to active compounds; pulmonary indications; gastrointestinal indications such as bowel cleansing, Irritable Bowel Syndrome (IBS), drug-induced (i.e. opioid) constipation, constipation/CIC of bedridden patients, acute infectious diarrhea, E. coli, cholera, viral gastroenteritis, rotavirus, modulation of malabsorption syndromes, pediatric diarrhea (viral, bacterial, protozoan), HIV, or short bowel syndrome; fertility indications such as sperm motility or sperm capacitation; female reproductive indications, cervical mucus/vaginal secretion viscosity (i.e. hostile cervical mucus); contraception, such as compounds that negatively affect sperm motility or cervical mucous quality relevant for sperm motility; dry mouth, dry eye, glaucoma, runny nose; or endocrine indications, i.e. pancreatic function in CF patients.

To identify a CFTR modulator, one can expose a novel cell or cell line of the invention to a test compound under conditions in which the CFTR would be expected to be functional and then detect a statistically significant change (e.g., p<0.05) in CFTR activity compared to a suitable control, e.g., cells that are not exposed to the test compound. Positive and/or negative controls using known agonists or antagonists and/or cells expressing different mutant CFTRs may also be used. In some embodiments, the CFTR activity to be detected and/or measured is membrane depolarization, change in membrane potential, or fluorescence resulting from such membrane changes. One of ordinary skill in the art would understand that various assay parameters may be optimized, e.g., signal to noise ratio.

In some embodiments, one or more cells or cell lines of the invention are exposed to a plurality of test compounds, for example, a library of test compounds. A library of test compounds can be screened using the cell lines of the invention to identify one or more modulators. The test compounds can be chemical moieties including small molecules, polypeptides, peptides, peptide mimetics, antibodies or antigen-binding portions thereof. In the case of antibodies, they may be non-human antibodies, chimeric antibodies, humanized antibodies, or fully human antibodies. The antibodies may be intact antibodies comprising a full complement of heavy and light chains or antigen-binding portions of any antibody, including antibody fragments (such as Fab, Fab′, F(ab′)₂, Fd, Fv, dAb, and the like), single chain antibodies (scFv), single domain antibodies, all or an antigen-binding portion of a heavy chain or light chain variable region.

In some embodiments, prior to exposure to a test compound, the cells or cell lines of the invention may be modified by pretreatment with, for example, enzymes, including mammalian or other animal enzymes, plant enzymes, bacterial enzymes, enzymes from lysed cells, protein modifying enzymes, lipid modifying enzymes, and enzymes in the oral cavity, gastrointestinal tract, stomach or saliva. Such enzymes can include, for example, kinases, proteases, phosphatases, glycosidases, oxidoreductases, transferases, hydrolases, lyases, isomerases, ligases and the like. Alternatively, the cells and cell lines may be exposed to the test compound first followed by treatment to identify compounds that alter the modification of the CFTR by the treatment.

In some embodiments, large compound collections are tested for CFTR modulating activity in a cell-based, functional, high-throughput screen (HTS), e.g., using a 96-well, 384-well, 1536-well or higher density well format. In some embodiments, a test compound or multiple test compounds including a library of test compounds may be screened using more than one cell or cell line of the invention. If multiple cells or cell lines, each expressing a different non-mutant CFTR or mutant CFTR are used, one can identify modulators that are effective on multiple forms of CFTR or alternatively, modulators that are specific for a particular mutant or non-mutant CFTR and that do not modulate other mutant CFTRs. In the case of a cell or cell line of the invention that expresses a human CFTR, one can expose the cells to a test compound to identify a compound that modulates CFTR activity (either increasing or decreasing) for use in the treatment of disease or condition characterized by undesired CFTR activity, or the decrease or absence of desired CFTR activity.

In certain embodiments, an assay for CFTR activity is performed using a cell or cell line expressing a CFTR mutant (see, e.g., Table 1 and Table 2), or a panel of mutants. In one embodiment, the panel also includes a cell or cell line that expresses wild type CFTR. In certain embodiments, a protein trafficking corrector is added to the assay. Such protein trafficking correctors include, but are not limited to: 1) Glycerol (see, e.g., Brown C R et al., Cell Stress and Chaperones (1996) v1(2):117-125); 2) DMSO (see, e.g., Brown C R et al., Cell Stress and Chaperones (1996) v1(2):117-125); 3) Deuterated water (D2O) (see, e.g., Brown C R et al., Cell Stress and Chaperones (1996) v1(2):117-125); 4) Methylamines such as Trimethylamine Oxide (TMAO) (see, e.g., Brown C R et al., Cell Stress and Chaperones (1996) v1(2):117-125); 5) Adamantyl sulfogalactosyl ceramide (adaSGC) (see, e.g., Park H J et al., Chemistry and Biology (2009) v16: 461-470); 6) Vasoactive intestinal peptide (VIP) (see, e.g., Journal of Biological Chemistry (1999) v112: 887-894); 7) Sodium Phenyl Butyrate (S-PBA) (see, e.g., Singh O V et al., Molecular and Cellular Proteomics (2008) v7:1099-1110); 8) VRT-325 (see, e.g., Wang Y et al., Journal of Biological Chemistry (2007) v282(46): 33247-33257); 9) VRT-422 (see, e.g., Van Goor F et al., American Journal of Physiology Lung Cell Molecular Physiology (2006) v290: L1117-1130); 10) Corrector 2b (see, e.g., Wang Y et al., Journal of Biological Chemistry (2007) v282(46): 33247-33257); 11) Corrector 3a (see, e.g., Wang Y et al., Journal of Biological Chemistry (2007) v282(46): 33247-33257); 12) Corrector 4a (see, e.g., Wang Y et al., Journal of Biological Chemistry (2007) v282(46): 33247-33257); 13) Curcumin (see, e.g., Robert R et al., Molecular Pharmacology (2008) v73: 478-489); 14) Sildenafil analog (KM11060) (see, Robert R et al., e.g., Molecular Pharmacology (2008) v73: 478-489); 15) Alanine, Glutamic Acid, Proline, GABA, Taruine, Sucrose, Trehalose, Myo-inositol, Arabitol, Mannitol, Mannose, Sucrose, Betaine, Glycerophosphorylcholine, Sarcosine (see, e.g., Welch W J et al., Cell Stress and Chaperones (1996) v1(2): 109-115); and 16) N-{2-[(2-methoxyphenyl)amino]-4′-methyl-4,5′-bi-1,3-thiazol-2′-yl}benzamide hydrobromide with the formula of

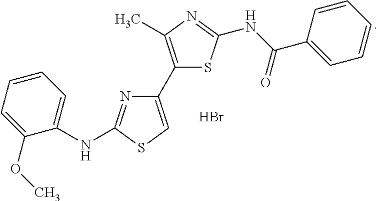

In certain embodiments, panels of cells or cell lines as described above can be used to test protein trafficking correctors. In certain embodiments, panels of cells or cell lines as described above can be used to screen for protein trafficking correctors.

In other embodiments, the assay of CFTR activity on a CFTR mutant is performed in the absence of a protein trafficking corrector. In some cases, the sensitivity of the CFTR activity assay is the same with or without the use of a protein trafficking corrector.

These and other embodiments of the invention may be further illustrated in the following non-limiting Examples.

### EXAMPLES

### Example 1

### Generating a Stable CFTR-Expressing Cell Line

### Generating Expression Constructs

Plasmid expression vectors that allowed streamlined cloning were generated based on pCMV-SCRIPT (Stratagene) and contained various necessary components for transcription and translation of a gene of interest, including: CMV and SV40 eukaryotic promoters; SV40 and HSV-TK polyadenylation sequences; multiple cloning sites; Kozak sequences; and drug resistance cassettes (i.e., puromycin). Ampicillin or neomycin resistance cassettes can also be used to substitute puromycin. A tag sequence (SEQ ID NO: 8) was then inserted into the multiple cloning site of the plasmid. A cDNA cassette encoding a human CFTR was then subcloned into the multiple cloning site upstream of the tag sequence, using Asc1 and Pac1 restriction endonucleases.

### Generating Cell Lines

### Step 1: Transfection

CHO cells were transfected with a plasmid encoding a human CFTR (SEQ ID NO: 1) using standard techniques. (Examples of reagents that may be used to introduce nucleic acids into host cells include, but are not limited to, LIPOFECTAMINE™, LIPOFECTAMINE™ 2000, OLIGOFECTAMINE™, TFX™ reagents, FUGENE® 6, DOTAP/DOPE, Metafectine or FECTURIN™.)

Although drug selection is optional to produce the cells or cell lines of this invention, we included one drug resistance marker in the plasmid (i.e., puromycin). The CFTR sequence was under the control of the CMV promoter. An untranslated sequence encoding a Target Sequence for detection by a signaling probe was also present along with the sequence encoding the drug resistance marker. The target sequence utilized was Target Sequence 2 (SEQ ID NO: 8), and in this example, the CFTR gene-containing vector comprised Target Sequence 2 (SEQ ID NO: 8).

### Step 2: Selection

Transfected cells were grown for 2 days in Ham's F12-FBS media (Sigma Aldrich, St Louis, Mo.) without antibiotics, followed by 10 days in 12.5 μg/ml puromycin-containing Ham's F12-FBS media. The cells were then transferred to Ham's F12-FBS media without antibiotics for the remainder of the time, prior to the addition of the signaling probe.

### Step 3: Cell Passaging

Following enrichment on antibiotic, cells were passaged 5-14 times in the absence of antibiotic selection to allow time for expression that was not stable over the selected period of time to subside.

### Step 4: Exposure of Cells to Fluorogenic Probes

Cells were harvested and transfected with Signaling Probe 2 (SEQ ID NO: 9) using standard techniques. (Examples of reagents that may be used to introduce nucleic acids into host cells include, but are not limited to, LIPOFECTAMINE™, LIPOFECTAMINE™ 2000, OLIGOFECTAMINE™, TFX™ reagents, FUGENE® 6, DOTAP/DOPE, Metafectine or FECTURIN™.) Signaling Probe 2 (SEQ ID NO: 9) bound Target Sequence 2 (SEQ ID NO: 8). The cells were then collected for analysis and sorted using a fluorescence activated cell sorter.

### Target Sequence Detected by Signaling Probe

Target Sequence 2

(SEQ ID NO: 8)
5′-GAAGTTAACCCTGTCGTTCTGCGAC-3′

### Signaling Probe

Signaling Probe 2 (supplied as 100 μM stock)

(SEQ ID NO: 9)
5′-CY5.5 GCGAGTCGCAGAACGACAGGGTTAACTTCCTCGC BHQ2-3′

BHQ2 in Signaling Probe 2 can be substituted with BHQ3 or a gold particle.

Target Sequence 2 and Signaling Probe 2 can be replaced by Target Sequence 1 and Signaling Probe 1, respectively.

Target Sequence 1

(SEQ ID NO: 3)
5′-GTTCTTAAGGCACAGGAACTGGGAC-3′

Signaling Probe 1 (supplied as 100 μM stock)

(SEQ ID NO: 6)
5′-Cy5 GCCAGTCCCAGTTCCTGTGCCTTAAGAACCTCGC BHQ2-3′

BHQ2 in Signaling Probe 1 can be substituted with BHQ3 or a gold particle.

In addition, a similar probe using a Quasar® Dye (BioSearch) with spectral properties similar to Cy5 is used in certain experiments against Target Sequence 1.

In some experiments, 5-MedC and 2-amino dA mixmers are used rather than DNA probes.

A non-targeting FAM labeled probe is also used as a loading control.

### Step 5: Isolation of Positive Cells

The cells were dissociated and collected for analysis and sorting using a fluorescence activated cell sorter (Beckman Coulter, Miami, Fla.). Standard analytical methods were used to gate cells fluorescing above background and to isolate individual cells falling within the gate into bar-coded 96-well plates. The following gating hierarchy was used:
coincidence gate→singlets gate→live gate→Sort gate in plot FAM vs. Cy5.5: 0.1-0.4% of cells according to standard procedures in the field.
   Step 6: Additional Cycles of Steps 1-5 and/or 3-5

Steps 1-5 and/or 3-5 were repeated to obtain a greater number of cells. Two rounds of steps 1-5 were performed, and for each of these rounds, two internal cycles of steps 3-5 were performed.

### Step 7: Estimation of Growth Rates for the Populations of Cells

The plates were transferred to a Microlab Star (Hamilton Robotics). Cells were incubated for 9 days in 100 μl of 1:1 mix of fresh complete growth media and 2 to 3 day-conditioned growth media, supplemented with 100 units/ml penicillin and 0.1 mg/ml streptomycin. Then the cells were dispersed by trypsinization once or twice to minimize clumps and later transferred to new 96-well plates. Plates were imaged to determine confluency of wells (Genetix). Each plate was focused for reliable image acquisition across the plate. Reported confluencies of greater than 70% were not relied upon. Confluency measurements were obtained on consecutive days between days 1 and 10 post-dispersal and used to calculate growth rates.

### Step 8: Binning Populations of Cells According to Growth Rate Estimates

Cells were binned (independently grouped and plated as a cohort) according to growth rate less than two weeks following the dispersal step in step 7. Each of the three growth bins was separated into individual 96 well plates; some growth bins resulted in more than one 96 well plate. Bins were calculated by considering the spread of growth rates and bracketing a high percentage of the total number of populations of cells. Bins were calculated to capture 12-16 hour differences in growth rate.

Cells can have doubling times from less than 1 day to more than 2 week. In order to process the most diverse clones that at the same time can be reasonably binned according to growth rate, it may be preferable to use 3-9 bins with a 0.25 to 0.7 day difference among the bins. One skilled in the art will appreciate that the tightness of the bins and number of bins can be adjusted for the particular situation and that the tightness and number of bins can be further adjusted if cells are synchronized for their cell cycle.

### Step 9: Replica Plating to Speed Parallel Processing and Provide Stringent Quality Control

The plates were incubated under standardized and fixed conditions (i.e., Ham's F12-FBS media, 37° C./5% CO2) without antibiotics. The plates of cells were split to produce 4 sets of 96 well plates (3 sets for freezing, 1 set for assay and passage). Distinct and independent tissue culture reagents, incubators, personnel, and carbon dioxide sources were used for each of the sets of the plates. Quality control steps were taken to ensure the proper production and quality of all tissue culture reagents: each component added to each bottle of media prepared for use was added by one designated person in one designated hood with only that reagent in the hood while a second designated person monitored to avoid mistakes. Conditions for liquid handling were set to eliminate cross contamination across wells. Fresh tips were used for all steps or stringent tip washing protocols were used. Liquid handling conditions were set for accurate volume transfer, efficient cell manipulation, washing cycles, pipetting speeds and locations, number of pipetting cycles for cell dispersal, and relative position of tip to plate.

### Step 10: Freezing Early Passage Stocks of Populations of Cells

Three sets of plates were frozen at −70 to −80° C. Plates in the set were first allowed to attain confluencies of 70 to 100%. Medium was aspirated and 90% FBS and 10% DMSO was added. The plates were individually sealed with Parafilm, individually surrounded by 1 to 5 cm of foam, and then placed into a −80° C. freezer.

### Step 11: Methods and Conditions for Initial Transformative Steps to Produce Viable, Stable and Functional (VSF) Cell Lines

The remaining set of plates was maintained as described in step 9. All cell splitting was performed using automated liquid handling steps, including media removal, cell washing, trypsin addition and incubation, quenching and cell dispersal steps.

### Step 12: Normalization Methods to Correct Any Remaining Variability of Growth Rates

The consistency and standardization of cell and culture conditions for all populations of cells was controlled. Differences across plates due to slight differences in growth rates were controlled by normalization of cell numbers across plates and occurred every 8 passages after the rearray. Populations of cells that were outliers were detected and eliminated.

### Step 13: Characterization of Population of Cells

The cells were maintained for 6 to 10 weeks post rearray in culture. During this time, we observed size, morphology, tendency towards microconfluency, fragility, response to trypsinization and average circularity post-trypsinization, or other aspects of cell maintenance such as adherence to culture plate surfaces and resistance to blow-off upon fluid addition as part of routine internal quality control to identify robust cells. Such benchmarked cells were then admitted for functional assessment.

### Step 14: Assessment of Potential Functionality of Populations of Cells Under VSF Conditions

Populations of cells were tested using functional criteria. Membrane potential dye kits (Molecular Devices, MDS) were used according to manufacturer's instructions.

Cells were tested at varying densities in 384-well plates (i.e., 12.5×10³ to 20×10³ cells/per well) and responses were analyzed. Time between cell plating and assay read was tested. Dye concentration was also tested. Dose response curves and Z′ scores were both calculated as part of the assessment of potential functionality.

The following steps (i.e., steps 15-18) can also be conducted to select final and back-up viable, stable, and functional cell lines.

### Step 15:

The functional responses from experiments performed at low and higher passage numbers are compared to identify cells with the most consistent responses over defined periods of time (e.g., 3-9 weeks). Other characteristics of the cells that change over time are also noted.

### Step 16:

Populations of cells meeting functional and other criteria are further evaluated to determine those most amenable to production of viable, stable and functional cell lines. Selected populations of cells are expanded in larger tissue culture vessels and the characterization steps described above are continued or repeated under these conditions. At this point, additional standardization steps, such as different cell densities; time of plating, length of cell culture passage; cell culture dishes format and coating; fluidics optimization, including speed and shear force; time of passage; and washing steps, are introduced for consistent and reliable passages.

In addition, viability of cells at each passage is determined. Manual intervention is increased and cells are more closely observed and monitored. This information is used to help identify and select final cell lines that retain the desired properties. Final cell lines and back-up cell lines are selected that show appropriate adherence/stickiness, growth rate, and even plating (lack of microconfluency) when produced following this process and under these conditions.

### Step 17: Establishment of Cell Banks

The low passage frozen stocks corresponding to the final cell line and back-up cell lines are thawed at 37° C., washed two times with Ham's F12-FBS and then incubated in Ham's F12-FBS. The cells are then expanded for a period of 2 to 4 weeks. Cell banks of clones for each final and back-up cell line are established, with 25 vials for each clonal cells being cryopreserved.

### Step 18:

At least one vial from the cell bank is thawed and expanded in culture. The resulting cells are tested to determine if they meet the same characteristics for which they are originally selected.

### Example 2

### Characterizing Stable Cell Lines for Native CFTR Function

We used a high-throughput compatible fluorescence membrane potential assay to characterize native CFTR function in the produced stable CFTR-expressing cell lines.

CHO cell lines stably expressing CFTR were maintained under standard cell culture conditions in Ham's F12 medium supplemented with 10% fetal bovine serum and glutamine. On the day before assay, the cells were harvested from stock plates and plated into black clear-bottom 384 well assay plates at a density that is sufficient to attain 90% confluency on the day of the assay. The assay plates were maintained in a 37° C. cell culture incubator under 5% CO₂ for 22-24 hours. The media was then removed from the assay plates and blue membrane potential dye (Molecular Devices Inc.) diluted in loading buffer (137 mM NaCl, 5 mM KCl, 1.25 mM CaCl₂, 25 mM HEPES, 10 mM glucose) was added and allowed to incubate for 1 hour at 37° C. The assay plates were then loaded on a fluorescent plate reader (Hamamatsu FDSS) and a cocktail of forskolin and IBMX dissolved in compound buffer (137 mM sodium gluconate, 5 mM potassium gluconate, 1.25 mM CaCl₂, 25 mM HEPES, 10 mM glucose) was added.

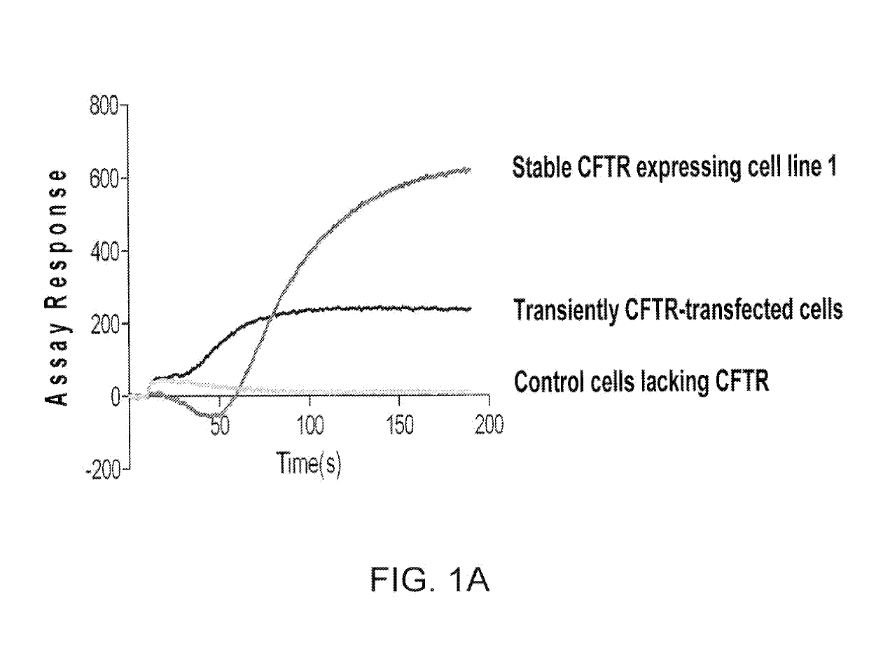

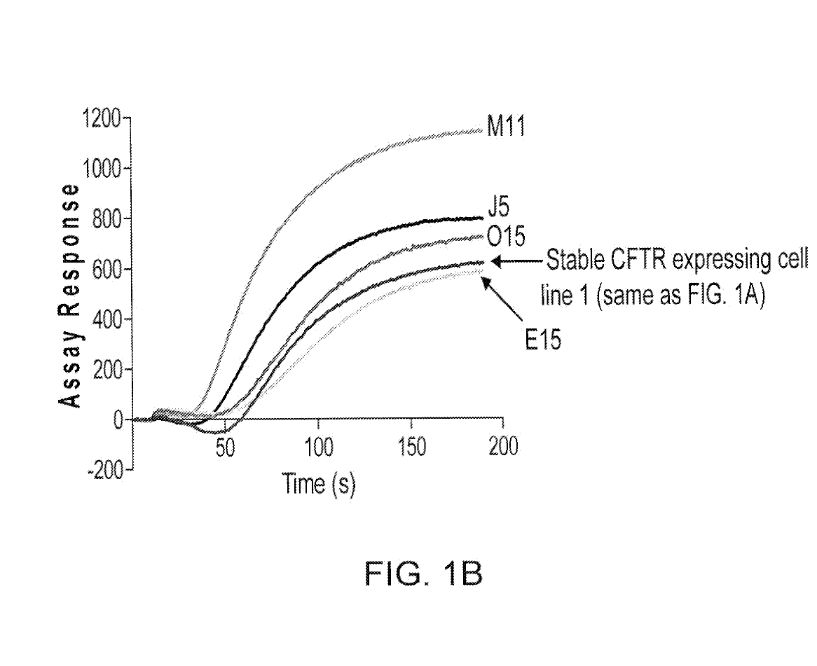

Representative data from the fluorescence membrane potential assay is presented in FIGS. 1A and 1B. The ion flux attributable to functional CFTR in stable CFTR-expressing CHO cell lines (cell line 1, M11, J5, E15, and O15) were all higher than control cells lacking CFTR as indicated by the assay response.

The ion flux attributable to functional CFTR in stable CFTR-expressing CHO cell lines (cell line 1, M11, J5, E15, and O15) were also all higher than transiently CFTR-transfected CHO cells (FIGS. 1A and 1B). The transiently CFTR-transfected cells were generated by plating CHO cells at 5-16 million per 10 cm tissue culture dish and incubating them for 18-20 hours before transfection. A transfection complex consisting of lipid transfection reagent and plasmids encoding CFTR was directly added to each dish. The cells were then incubated at 37° C. in a CO₂ incubator for 6-12 hours. After incubation, the cells were lifted, plated into black clear-bottom 384 well assay plates, and assayed for function using the above-described fluorescence membrane potential assay.

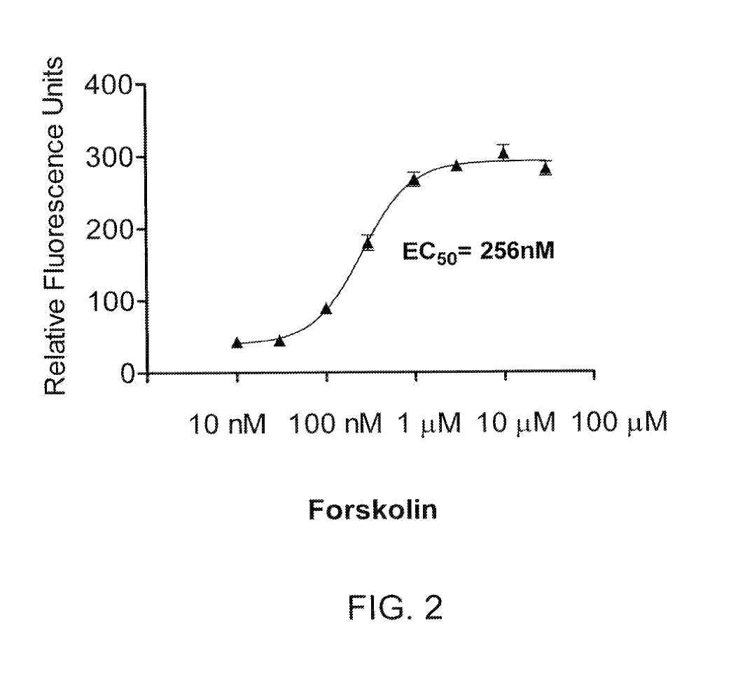

For forskolin dose-response experiments, cells of the produced stable CFTR-expressing cell lines, plated at a density of 15,000 cells/well in a 384-well plate were challenged with increasing concentration of forskolin, a known CFTR agonist. The cellular response as a function of changes in cell fluorescence was monitored over time by a fluorescent plate reader (Hamamatsu FDSS). Data were then plotted as a function of forskolin concentration and analyzed using non-linear regression analysis using GraphPad Prism 5.0 software, resulting in an EC₅₀ of 256 nM (FIG. 2). The produced CFTR-expressing cell line shows a EC₅₀ value of forskolin within the ranges of EC₅₀ of forskolin previously reported in other cell lines (between 250 and 500 nM) (Galietta et al., Am J Physiol Cell Physiol. 281(5): C1734-1742 (2001)), indicating the potency of the clone.

### Example 3

### Determination of Z′ Value for CFTR Cell-Based Assay

Z′ value for the produced stable CFTR-expressing cell line was calculated using a high-throughput compatible fluorescence membrane potential assay. The fluorescence membrane potential assay protocol was performed substantially according to the protocol in Example 2. Specifically for the Z′ assay, 24 positive control wells in a 384-well assay plate (plated at a density of 15,000 cells/well) were challenged with a CFTR activating cocktail of forskolin and IBMX. An equal number of wells were challenged with vehicle alone and containing DMSO (in the absence of activators). Cell responses in the two conditions were monitored using a fluorescent plate reader (Hamamatsu FDSS). Mean and standard deviations in the two conditions were calculated and Z′ was computed using the method disclosed in Zhang et al., J Biomol Screen, 4(2): 67-73, (1999). The Z′ value of the produced stable CFTR-expressing cell line was determined to be higher than or equal to 0.82.

### Example 4

### High-Throughput Screening and Identification of CFTR Modulators

A high-throughput compatible fluorescence membrane potential assay is used to screen and identify CFTR modulator. On the day before assay, the cells are harvested from stock plates into growth media without antibiotics and plated into black clear-bottom 384 well assay plates. The assay plates are maintained in a 37° C. cell culture incubator under 5% CO₂ for 19-24 hours. The media is then removed from the assay plates and blue membrane potential dye (Molecular Devices Inc.) diluted in load buffer (137 mM NaCl, 5 mM KCl, 1.25 mM CaCl₂, 25 mM HEPES, 10 mM glucose) is added and the cells are incubated for 1 hr at 37° C. Test compounds are solubilized in dimethylsulfoxide, diluted in assay buffer (137 mM sodium gluconate, 5 mM potassium gluconate, 1.25 mM CaCl₂, 25 mM HEPES, 10 mM glucose) and then loaded into 384 well polypropylene micro-titer plates. The cell and compound plates are loaded into a fluorescent plate reader (Hamamatsu FDSS) and run for 3 minutes to identify test compound activity. The instrument adds a forskolin solution at a concentration of 300 nM-1 μM to the cells to allow either modulator or blocker activity of the previously added compounds to be observed. The activity of the compound is determined by measuring the change in fluorescence produced following the addition of the test compounds to the cells and/or following the subsequent agonist addition.

### Example 5

### Characterizing Stable CFTR-Expressing Cell Lines for Native CFTR Function Using Short-Circuit Current Measurements

Ussing chamber experiments are performed 7-14 days after plating CFTR-expressing cells (primary or immortalized epithelial cells including but not limited to lung and intestinal) on culture inserts (Snapwell, Corning Life Sciences). Cells on culture inserts are rinsed, mounted in an Ussing type apparatus (EasyMount Chamber System, Physiologic Instruments) and bathed with continuously gassed Ringer solution (5% CO₂ in O₂, pH 7.4) maintained at 37° C. containing 120 mM NaCl, 25 mM NaHCO₃, 3.3 mM KH₂PO₄, 0.8 mM K₂HPO₄, 1.2 mM CaCl₂, 1.2 mM MgCl₂, and 10 mM glucose. The hemichambers are connected to a multichannel voltage and current clamp (VCC-MC8 Physiologic Instruments). Electrodes [agar bridged (4% in 1 M KCl) Ag—AgCl] are used and the inserts are voltage clamped to 0 mV. Transepithelial current, voltage and resistance are measured every 10 seconds for the duration of the experiment. Membranes with a resistance of <200 mΩs are discarded.

### Example 6

### Characterizing Stable CFTR-Expressing Cell Lines for Native CFTR Function Using Electrophysiological Assay

While both manual and automated electrophysiology assays have been developed and both can be applied to assay the native CFTR function, described below is the protocol for manual patch clamp experiments.

Cells are seeded at loe densities and are used 2-4 days after plating. Borosilicate glass pipettes are fire-polished to obtain tip resistances of 2-4 mega Ω. Currents are sampled and low pass filtered. The extracellular (bath) solution contains: 150 mM NaCl, 1 mM CaCl₂, 1 mM MgCl₂, 10 mM glucose, 10 mM mannitol, and 10 mM TES, pH 7.4. The pipette solution contains: 120 mM CsCl, 1 mM MgCl₂, 10 mM TEA-Cl, 0.5 mM EGTA, 1 mM Mg-ATP, and 10 mM HEPES (pH 7.3). Membrane conductances are monitored by alternating the membrane potential between −80 mV and −100 mV. Current-voltage relationships are generated by applying voltage pulses between −100 mV and +100 mV in 20-mV steps.

### Example 7

### Generating a Stable CFTR-ΔF508 Expressing Cell Line

### Generating Expression Constructs

Plasmid expression vectors that allowed streamlined cloning were generated based on pCMV-SCRIPT (Stratagene) and contained various necessary components for transcription and translation of a gene of interest, including: CMV and SV40 eukaryotic promoters; SV40 and HSV-TK polyadenylation sequences; multiple cloning sites; Kozak sequences; and drug resistance cassettes (i.e., puromycin). Ampicillin or neomycin resistance cassettes can also be used to substitute puromycin. A tag sequence (SEQ ID NO: 8) was then inserted into the multiple cloning site of the plasmid. A cDNA cassette encoding a human CFTR was then subcloned into the multiple cloning site upstream of the tag sequence, using Asc1 and Pac1 restriction endonucleases. A site-directed mutagenesis (Stratagene) was then used to delete a single phenylalanine amino-acid at position 508 to generate plasmid encoding human CFTR-ΔF508 (SEQ ID NO: 7). The above-described mutagenesis method is compatible with high-throughput generation of any number of various CFTR alleles (either currently known or as may become known in the future).

### Generating Cell Lines

### Step 1: Transfection

CHO cells were transfected with a plasmid encoding a human CFTR-ΔF508 (SEQ ID NO: 7) using standard techniques. (Examples of reagents that may be used to introduce nucleic acids into host cells include, but are not limited to, LIPOFECTAMINE™, LIPOFECTAMINE™ 2000, OLIGOFECTAMINE™, TFX™ reagents, FUGENE® FUGENE 6, DOTAP/DOPE, Metafectine or FECTURIN™.)

Although drug selection is optional to produce the cells or cell lines of this invention, we included one drug resistance marker in the plasmid (i.e., puromycin). The CFTR-ΔF508 sequence was under the control of the CMV promoter. An untranslated sequence encoding a Target Sequence for detection by a signaling probe was also present along with the sequence encoding the drug resistance marker. The target sequence utilized was Target Sequence 2 (SEQ ID NO: 8), and in this example, the CFTR-ΔF508-containing vector comprised Target Sequence 2 (SEQ ID NO: 8).

### Step 2: Selection

Transfected cells were grown for 2 days in Ham's F12-FBS media (Sigma Aldrich, St. Louis, Mo.) without antibiotics, followed by 10 days in 12.5 μg/ml puromycin-containing Ham's F12-FBS media. The cells were then transferred to Ham's F12-FBS media without antibiotics for the remainder of the time, prior to the addition of the signaling probe.

### Step 3: Cell Passaging

Following enrichment on antibiotic, cells were passaged 5-14 times in the absence of antibiotic selection to allow time for expression that was not stable over the selected period of time to subside.

### Step 4: Exposure of Cells to Fluorogenic Probes

Cells were harvested and transfected with Signaling Probe 2 (SEQ ID NO: 9) using standard techniques. (Examples of reagents that may be used to introduce nucleic acids into host cells include, but are not limited to, LIPOFECTAMINE™, LIPOFECTAMINE™ 2000, OLIGOFECTAMINE™, TFX™ reagents, FUGENE® 6, DOTAP/DOPE, Metafectine or FECTURIN™.) Signaling Probe 2 (SEQ ID NO: 9) bound Target Sequence 2 (SEQ ID NO: 8). The cells were then collected for analysis and sorted using a fluorescence activated cell sorter.

### Target Sequence Detected by Signaling Probe

Target Sequence 2

(SEQ ID NO: 8)
5′-GAAGTTAACCCTGTCGTTCTGCGAC-3′

### Signaling Probe

Signaling Probe 2 (supplied as 100 μM stock)

(SEQ ID NO: 9)
5′-CY5.5 GCGAGTCGCAGAACGACAGGGTTAACTTCCTCGC BHQ2-3′

BHQ2 in Signaling Probe 2 can be substituted with BHQ3 or a gold particle.

Target Sequence 2 and Signaling Probe 2 can be replaced by Target Sequence 1 and Signaling Probe 1, respectively.

Target Sequence 1

(SEQ ID NO: 3)
5′-GTTCTTAAGGCACAGGAACTGGGAC-3′

Signaling Probe 1 (supplied as 100 μM stock)

(SEQ ID NO: 6)
5′-Cy5 GCCAGTCCCAGTTCCTGTGCCTTAAGAACCTCGC BHQ2-3′

BHQ2 in Signaling Probe 1 can be substituted with BHQ3 or a gold particle.

In addition, a similar probe using a Quasar® Dye (BioSearch) with spectral properties similar to Cy5 is used in certain experiments against Target Sequence 1.

In some experiments, 5-MedC and 2-amino dA mixmers are used rather than DNA probes.

A non-targeting FAM labeled probe is also used as a loading control.

### Step 5: Isolation of Positive Cells

The cells were dissociated and collected for analysis and sorting using a fluorescence activated cell sorter (Beckman Coulter, Miami, Fla.). Standard analytical methods were used to gate cells fluorescing above background and to isolate individual cells falling within the gate into bar-coded 96-well plates. The following gating hierarchy was used:
coincidence gate→singlets gate→live gate→Sort gate in plot FAM vs. Cy5.5: 0.1-0.5% of cells according to standard procedures in the field.
   Step 6: Additional Cycles of Steps 1-5 and/or 3-5

Steps 1-5 and/or 3-5 were repeated to obtain a greater number of cells. Two rounds of steps 1-5 were performed, and for each of these rounds, two internal cycles of steps 3-5 were performed.

### Step 7: Estimation of Growth Rates for the Populations of Cells

The plates were transferred to a Microlab Star (Hamilton Robotics). Cells were incubated for 9 days in 100 μl of 1:1 mix of fresh complete growth media and 2 to 3 day-conditioned growth media, supplemented with 100 units/ml penicillin and 0.1 mg/ml streptomycin. Then the cells were dispersed by trypsinization once or twice to minimize clumps and later transferred to new 96-well plates. Plates were imaged to determine confluency of wells (Genetix). Each plate was focused for reliable image acquisition across the plate. Reported confluencies of greater than 70% were not relied upon. Confluency measurements were obtained on consecutive days between days 1 and 10 post-dispersal and used to calculate growth rates.

### Step 8: Binning Populations of Cells According to Growth Rate Estimates

Cells were binned (independently grouped and plated as a cohort) according to growth rate less than two weeks following the dispersal step in step 7. Each of the three growth bins was separated into individual 96 well plates; some growth bins resulted in more than one 96 well plate. Bins were calculated by considering the spread of growth rates and bracketing a high percentage of the total number of populations of cells. Bins were calculated to capture 12-16 hour differences in growth rate.

Cells can have doubling times from less than 1 day to more than 2 week. In order to process the most diverse clones that at the same time can be reasonably binned according to growth rate, it may be preferable to use 3-9 bins with a 0.25 to 0.7 day doubling time per bin. One skilled in the art will appreciate that the tightness of the bins and number of bins can be adjusted for the particular situation and that the tightness and number of bins can be further adjusted if cells are synchronized for their cell cycle.

### Step 9: Replica Plating to Speed Parallel Processing and Provide Stringent Quality Control

The plates were incubated under standardized and fixed conditions (i.e., Ham's F12-FBS media, 37° C./5% CO2) without antibiotics. The plates of cells were split to produce 2 sets of 96 well plates (1 set for freezing, 1 set for assay and passage). Distinct and independent tissue culture reagents, incubators, personnel, and carbon dioxide sources were used for each of the sets of the plates. Quality control steps were taken to ensure the proper production and quality of all tissue culture reagents: each component added to each bottle of media prepared for use was added by one designated person in one designated hood with only that reagent in the hood while a second designated person monitored to avoid mistakes. Conditions for liquid handling were set to eliminate cross contamination across wells. Fresh tips were used for all steps or stringent tip washing protocols were used. Liquid handling conditions were set for accurate volume transfer, efficient cell manipulation, washing cycles, pipetting speeds and locations, number of pipetting cycles for cell dispersal, and relative position of tip to plate.

### Step 10: Freezing Early Passage Stocks of Populations of Cells

One set of plate was frozen at −70 to −80° C. Plates were first allowed to attain confluencies of 70 to 100%. Medium was aspirated and 90% FBS and 10% DMSO was added. The plates were individually sealed with Parafilm, individually surrounded by 1 to 5 cm of foam, and then placed into a −80° C. freezer.

### Step 11: Methods and Conditions for Initial Transformative Steps to Produce Viable, Stable and Functional (VSF) Cell Lines

The remaining set of plates was maintained as described in step 9. All cell splitting was performed using automated liquid handling steps, including media removal, cell washing, trypsin addition and incubation, quenching and cell dispersal steps.

### Step 12: Normalization Methods to Correct Any Remaining Variability of Growth Rates

The consistency and standardization of cell and culture conditions for all populations of cells are controlled. Differences across plates due to slight differences in growth rates are controlled by normalization of cell numbers across plates and occurred every 8 passages after the re-array. Populations of cells that are outliers are detected and eliminated.

### Step 13: Characterization of Population of Cells

The cells were maintained for 6 to 10 weeks post rearray in the culture. During this time, we observed size, morphology, tendency towards microconfluency, fragility, response to trypsinization and average circularity post-trypsinization, or other aspects of cell maintenance such as adherence to culture plate surfaces and resistance to blow-off upon fluid addition as part of routine internal quality control to identify robust cells. Such benchmarked cells were then admitted for functional assessment.

### Step 14: Assessment of Potential Functionality of Populations of Cells Under VSF Conditions

Populations of cells were tested for receptor function using a high throughput compatible fluorescence based membrane potential dye kit (Molecular Devices, MDS) according to manufacturer's instructions.

Population of CHO cells stably expressing CFTR-ΔF508 were maintained under standard cell culture conditions in Ham's F12 medium supplemented with 10% fetal bovine serum and glutamine. On the day before assay, the cells were harvested from stock plates. The cells were plated into black clear-bottom 384 well assay plates at a density that was sufficient to attain 90% confluency on the day of the assay, with or without a protein trafficking corrector, Chembridge compound #5932794 (Chembridge, San Diego, Calif.) (Yoo et al., (2008) Bioorganic & Medicinal Chemistry Letters; 18(8): 2610-2614). This compound is N-{2-[(2-methoxyphenyl)amino]-4′-methyl-4,5′-bi-1,3-thiazol-2′-yl}benzamide hydrobromide, and has the formula of

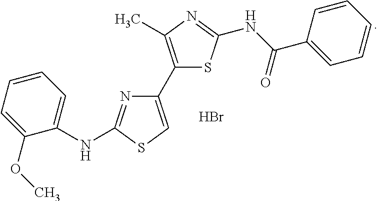

The assay plates were maintained in a 37° C. cell culture incubator under 5% CO₂ for 22-24 hours. The media was then removed from the assay plates and membrane potential dye diluted in loading buffer (137 mM NaCl, 5 mM KCl, 1.25 mM CaCl₂, 25 mM HEPES, 10 mM glucose) (blue or AnaSpec, Molecular Devices Inc.) was added, with or without a quencher of the membrane potential dye, and was allowed to incubate for 1 hour at 37° C. The quencher can be any quencher well known in the art, e.g., Dipicrylamine (DPA), Acid Violet 17 (AV17), Diazine Black (DB), HLB30818, Trypan Blue, Bromophenol Blue, HLB30701, HLB30702, HLB30703, Nitrazine Yellow, Nitro Red, DABCYL (Molecular Probes), QSY (Molecular Probes), metal ion quenchers (e.g., Co²⁺, Ni²⁺, Cu²⁺), and iodide ion.

The assay plates were then loaded on a fluorescent plate reader (Hamamatsu FDSS) and a cocktail of forskolin and IBMX dissolved in compound buffer (137 mM sodium gluconate, 5 mM potassium gluconate, 1.25 mM CaCl₂, 25 mM HEPES, 10 mM glucose) was added.

Representative data from the fluorescence membrane potential assay are presented in FIGS. 3A-3F. The ion flux attributable to functional CFTR-ΔF508 in stable CFTR-ΔF508 expressing CHO cell lines were identified by comparing the receptor's response to forskolin (30 μM)+IBMX (100 μM) cocktail against DMSO+Buffer controls (FIGS. 3A-3F) either in the presence or absence of the protein trafficking corrector—Chembridge compound #5932794. FIGS. 3A and 3B show responding and non-responding (control) clones assayed using blue membrane potential dye in the presence of the protein trafficking corrector (15-25 μM); FIGS. 3C and 3D show responding and non-responding (control) clones assayed using AnaSpec membrane potential dye in the presence of the protein trafficking corrector (15-25 μM). FIGS. 3E and 3F show responding and non-responding (control) clones assayed using AnaSpec membrane potential dye in the absence of the protein trafficking corrector.

Cells will be tested at varying densities in 384-well plates (i.e., 12.5×10³ to 20×10³ cells/per well) and responses will be analyzed. Time between cell plating and assay read will be tested. Dye concentration will also be tested. Dose response curves and Z′ scores can be calculated as part of the assessment of potential functionality.

The following steps (i.e., steps 15-18) can also be conducted to select final and back-up viable, stable, and functional cell lines.

### Step 15:

The functional responses from experiments performed at low and higher passage numbers are compared to identify cells with the most consistent responses, over defined periods of time (e.g., 3-9 weeks). Other characteristics of the cells that change over time are also noted.

### Step 16:

Populations of cells meeting functional and other criteria will be further evaluated to determine those most amenable to production of viable, stable and functional cell lines. Selected populations of cells will be expanded in larger tissue culture vessels and the characterization steps described above will be continued or repeated under these conditions. At this point, additional standardization steps, such as different cell densities; time of plating, length of cell culture passage; cell culture dishes format—(note: not explored); fluidics optimization, including speed and shear force; time of passage; and washing steps, will be introduced for consistent and reliable passages.

In addition, viability of cells at each passage will be determined. Manual intervention will be increased and cells will be more closely observed and monitored. This information is used to help identify and select final cell lines that retain the desired properties. Final cell lines and back-up cell lines will be selected that show appropriate adherence/stickiness, growth rate, and even plating (lack of microconfluency) when produced following this process and under these conditions.

### Step 17: Establishment of Cell Banks

The low passage frozen stocks corresponding to the final cell line and back-up cell lines will be thawed at 37° C., washed once with Ham's F12-FBS and then incubated in Ham's F12-FBS. The cells will be then expanded for a period of 2 to 4 weeks. Cell banks of clones for each final and back-up cell line will be established, with 25 vials for each clonal cells being cryopreserved.

### Step 18:

At least one vial from the cell bank will be thawed and expanded in culture. The resulting cells will be tested to determine if they meet the same characteristics for which they are originally selected.

### Example 8

### Characterizing Stable Cell Lines for CFTR-ΔF508 Function

We will use a high-throughput compatible fluorescence membrane potential assay to characterize CFTR-ΔF508 function in the produced stable CFTR-ΔF508 expressing cell lines.

CHO cell lines stably expressing CFTR-ΔF508 will be maintained under standard cell culture conditions in Ham's F12 medium supplemented with 10% fetal bovine serum and glutamine. On the day before assay, the cells will be harvested from stock plates and plated into black clear-bottom 384 well assay plates in the presence or absence of a protein trafficking corrector (e.g., Chembridge compound #5932794, N-{2-[(2-methoxyphenyl)amino]-4′-methyl-4,5′-bi-1,3-thiazol-2′-yl}benzamide hydrobromide). The assay plates will be maintained in a 37° C. cell culture incubator under 5% CO₂ for 22-24 hours. The media will be then removed from the assay plates and blue membrane potential dye (Molecular Devices Inc.) diluted in loading buffer (137 mM NaCl, 5 mM KCl, 1.25 mM CaCl₂, 25 mM HEPES, 10 mM glucose) will be added and allowed to incubate for 1 hour at 37° C. The assay plates will be then loaded on a fluorescent plate reader (Hamamatsu FDSS) and a cocktail of forskolin and IBMX dissolved in compound buffer (137 mM sodium gluconate, 5 mM potassium gluconate, 1.25 mM CaCl₂, 25 mM HEPES, 10 mM glucose) will be added. Stable cell-lines expressing CFTR-ΔF508 protein will be identified by measuring the change in fluorescence produced following the addition of the agonist cocktail.

Stable cell lines expressing the CFTR-ΔF508 protein will be then characterized with increasing doses of forskolin. For forskolin dose-response experiments, cells of the produced stable CFTR-ΔF508 expressing cell lines, plated at a density of 15,000 cells/well in a 384-well plate will be challenged with increasing concentrations of forskolin, a CFTR agonist. The cellular response as a function of changes in cell fluorescence will be monitored over time by a fluorescent plate reader (Hamamatsu FDSS). Data will be then plotted as a function of forskolin concentration and analyzed using non-linear regression analysis using GraphPad Prism 5.0 software to determine the EC50 value.

### Example 9

### Determination of Z′ Value for CFTR-ΔF508 Cell-Based Assay

Z′ value for the produced stable CFTR-ΔF508 expressing cell line will be calculated using a high-throughput compatible fluorescence membrane potential assay. The fluorescence membrane potential assay protocol will be performed substantially according to the protocol in Example 8. Specifically for the Z′ assay, 24 positive control wells in a 384-well assay plate (plated at a density of 15,000 cells/well) will be challenged with a CFTR activating cocktail of forskolin and IBMX. An equal number of wells will be challenged with vehicle alone and containing DMSO (in the absence of activators). The assay can be performed in the presence or absence of a protein trafficking corrector (e.g., Chembridge compound #5932794, N-{2-[(2-methoxyphenyl)amino]-4′-methyl-4,5′-bi-1,3-thiazol-2′-yl}benzamide hydrobromide). Cell responses in the two conditions will be monitored using a fluorescent plate reader (Hamamatsu FDSS). Mean and standard deviations in the two conditions will be calculated and Z′ was computed using the method disclosed in Zhang et al., J Biomol Screen, 4(2): 67-73 (1999).

### Example 10

### High-Throughput Screening and Identification of CFTR-ΔF508 Modulators

A high-throughput compatible fluorescence membrane potential assay will be used to screen and identify CFTR-ΔF508 modulator(s). Modulating compounds may either enhance protein trafficking to the cell surface or modulate CFTR-ΔF508 agonists (for example, Forskolin) by increasing or decreasing the agonist activity. On the day before assay, the cells will be harvested from stock plates into growth media without antibiotics and plated into black clear-bottom 384 well assay plates in the presence or absence of a protein trafficking corrector (e.g., Chembridge compound #5932794—N-{2-[(2-methoxyphenyl)amino]-4′-methyl-4,5′-bi-1,3-thiazol-2′-yl}benzamide hydrobromide). The assay plates will be maintained in a 37° C. cell culture incubator under 5% CO₂ for 19-24 hours. The media will be then removed from the assay plates and blue membrane potential dye (Molecular Devices Inc.) diluted in load buffer (137 mM NaCl, 5 mM KCl, 1.25 mM CaCl₂, 25 mM HEPES, 10 mM glucose) will be added and the cells will be incubated for 1 hr at 37° C. Test compounds will be solubilized in dimethylsulfoxide, diluted in assay buffer (137 mM sodium gluconate, 5 mM potassium gluconate, 1.25 mM CaCl₂, 25 mM HEPES, 10 mM glucose) and then loaded into 384 well polypropylene micro-titer plates. The cell and compound plates will be loaded into a fluorescent plate reader (Hamamatsu FDSS) and run for 3 minutes to identify test compound activity. The instrument will then add a forskolin solution at a concentration of 300 nM-1 μM to the cells to allow either modulator or blocker activity of the previously added compounds to be observed. The activity of the compound will be determined by measuring the change in fluorescence produced following the addition of the test compounds to the cells and/or following the subsequent agonist addition.

For identification of compounds that may promote cell surface trafficking of the CFTR-ΔF508 protein on the day before assay, the cells will be harvested from stock plates into growth media without antibiotics and plated into black clear-bottom 384 well assay plates in the presence of the test compounds for a period of 24 hours. Some wells in the 384 well plate will not receive any test compound as negative controls, while others wells in the 384 well plates will receive a protein trafficking corrector (e.g., Chembridge compound #5932794, N-{2-[(2-methoxyphenyl)amino]-4′-methyl-4,5′-bi-1,3-thiazol-2′-yl}benzamide hydrobromide) and serve as positive controls. The assay plates will be maintained in a 37° C. cell culture incubator under 5% CO₂ for 19-24 hours. The media will be then removed from the assay plates and blue membrane potential dye (Molecular Devices Inc.) diluted in load buffer (137 mM NaCl, 5 mM KCl, 1.25 mM CaCl₂, 25 mM HEPES, 10 mM glucose) will be added and the cells will be incubated for 1 hr at 37° C. The assay plates will be then loaded on a fluorescent plate reader (Hamamatsu FDSS) and a cocktail of forskolin and IBMX dissolved in compound buffer (137 mM sodium gluconate, 5 mM potassium gluconate, 1.25 mM CaCl₂, 25 mM HEPES, 10 mM glucose) will be added. The activity of the test compounds will be determined by measuring the change in fluorescence produced following the addition of the agonist cocktail (i.e. forskolin+IBMX).

### Example 11

### Characterizing Stable CFTR-ΔF508 Expressing Cell Lines for CFTR-ΔF508 Function Using Short-Circuit Current Measurements

Ussing chamber experiments will be performed 7-14 days after plating CFTR-ΔF508 expressing cells (e.g., primary or immortalized epithelial cells including but not limited to lung and intestinal cells) on culture inserts (Snapwell, Corning Life Sciences). Cells on culture inserts will be rinsed, mounted in an Ussing type apparatus (EasyMount Chamber System, Physiologic Instruments) and bathed with continuously gassed Ringer solution (5% CO₂ in O₂, pH 7.4) maintained at 37° C. containing 120 mM NaCl, 25 mM NaHCO₃, 3.3 mM KH₂PO₄, 0.8 mM K₂HPO₄, 1.2 mM CaCl₂, 1.2 mM MgCl₂, and 10 mM glucose. The hemichambers will be connected to a multichannel voltage and current clamp (VCC-MC8 Physiologic Instruments). Electrodes [agar bridged (4% in 1 M KCl) Ag—AgCl] will be used and the inserts will be voltage clamped to 0 mV. Transepithelial current, voltage, and resistance will be measured every 10 seconds for the duration of the experiment. Membranes with a resistance of <200 mΩs will be discarded.

### Example 12

### Characterizing Stable CFTR-ΔF508 Expressing Cell Lines for CFTR-ΔF508 Function Using Electrophysiological Assay

While both manual and automated electrophysiology assays have been developed and both can be applied to characterize stable CFTR-ΔF508 expressing cell lines for CFTR-ΔF508 function, described below is the protocol for manual patch clamp experiments.

Cells are seeded at low densities and are used 2-4 days after plating. Borosilicate glass pipettes are fire-polished to obtain tip resistances of 2-4 mega Ω. Currents will be sampled and low pass filtered. The extracellular (bath) solution will contain: 150 mM NaCl, 1 mM CaCl₂, 1 mM MgCl₂, 10 mM glucose, 10 mM mannitol, and 10 mM TES, pH 7.4. The pipette solution will contain: 120 mM CsCl, 1 mM MgCl₂, 10 mM TEA-Cl, 0.5 mM EGTA, 1 mM Mg-ATP, and 10 mM HEPES (pH 7.3). Membrane conductances will be monitored by alternating the membrane potential between −80 mV and −100 mV. Current-voltage relationships will be generated by applying voltage pulses between −100 mV and +100 mV in 20-mV steps.

LISTING OF SEQUENCES
Homo sapiens (H. s.) cystic fibrosis transmembrane conductance regulator
(CFTR) nucleotide sequence (SEQ ID NO: 1):
atgcagaggtcgcctctggaaaaggccagcgttgtctccaaactttttttcagctggaccagaccaattttgaggaaaggatacag
acagcgcctggaattgtcagacatataccaaatcccttctgttgattctgctgacaatctatctgaaaaattggaaagagaatggga
tagagagctggcttcaaagaaaaatcctaaactcattaatgcccttcggcgatgttttttctggagatttatgttctatggaatctttttat
atttaggggaagtcaccaaagcagtacagcctctcttactgggaagaatcatagcttcctatgacccggataacaaggaggaac
gctctatcgcgatttatctaggcataggcttatgccttctctttattgtgaggacactgctcctacacccagccatttttggccttcatca
cattggaatgcagatgagaatagctatgtttagtttgatttataagaagactttaaagctgtcaagccgtgttctagataaaataagta
ttggacaacttgttagtctcctttccaacaacctgaacaaatttgatgaaggacttgcattggcacatttcgtgtggatcgctcctttgc
aagtggcactcctcatggggctaatctgggagttgttacaggcgtctgccttctgtggacttggtttcctgatagtccttgccctttttc
aggctgggctagggagaatgatgatgaagtacagagatcagagagctgggaagatcagtgaaagacttgtgattacctcagaa
atgattgaaaatatccaatctgttaaggcatactgctgggaagaagcaatggaaaaaatgattgaaaacttaagacaaacagaact
gaaactgactcggaaggcagcctatgtgagatacttcaatagctcagccttcttcttctcagggttctttgtggtgtttttatctgtgctt
ccctatgcactaatcaaaggaatcatcctccggaaaatattcaccaccatctcattctgcattgttctgcgcatggcggtcactcgg
caatttccctgggctgtacaaacatggtatgactctcttggagcaataaacaaaatacaggatttcttacaaaagcaagaatataag
acattggaatataacttaacgactacagaagtagtgatggagaatgtaacagccttctgggaggagggatttggggaattatttga
gaaagcaaaacaaaacaataacaatagaaaaacttctaatggtgatgacagcctcttcttcagtaatttctcacttcttggtactcct
gtcctgaaagatattaatttcaagatagaaagaggacagttgttggcggttgctggatccactggagcaggcaagacttcacttct
aatggtgattatgggagaactggagccttcagagggtaaaattaagcacagtggaagaatttcattctgttctcagttttcctggatt
atgcctggcaccattaaagaaaatatcatctttggtgtttcctatgatgaatatagatacagaagcgtcatcaaagcatgccaactag
aagaggacatctccaagtttgcagagaaagacaatatagttcttggagaaggtggaatcacactgagtggaggtcaacgagcaa
gaatttctttagcaagagcagtatacaaagatgctgatttgtatttattagactctccttttggatacctagatgttttaacagaaaaaga
aatatttgaaagctgtgtctgtaaactgatggctaacaaaactaggattttggtcacttctaaaatggaacatttaaagaaagctgac
aaaatattaattttgcatgaaggtagcagctatttttatgggacattttcagaactccaaaatctacagccagactttagctcaaaact
catgggatgtgattctttcgaccaatttagtgcagaaagaagaaattcaatcctaactgagaccttacaccgtttctcattagaagga
gatgctcctgtctcctggacagaaacaaaaaaacaatcttttaaacagactggagagtttggggaaaaaaggaagaattctattct
caatccaatcaactctatacgaaaattttccattgtgcaaaagactcccttacaaatgaatggcatcgaagaggattctgatgagcc
tttagagagaaggctgtccttagtaccagattctgagcagggagaggcgatactgcctcgcatcagcgtgatcagcactggccc
cacgcttcaggcacgaaggaggcagtctgtcctgaacctgatgacacactcagttaaccaaggtcagaacattcaccgaaaga
caacagcatccacacgaaaagtgtcactggcccctcaggcaaacttgactgaactggatatatattcaagaaggttatctcaaga
aactggcttggaaataagtgaagaaattaacgaagaagacttaaaggagtgcttttttgatgatatggagagcataccagcagtga
ctacatggaacacataccttcgatatattactgtccacaagagcttaatttttgtgctaatttggtgcttagtaatttttctggcagaggt
ggctgcttctttggttgtgctgtggctccttggaaacactcctcttcaagacaaagggaatagtactcatagtagaaataacagctat
gcagtgattatcaccagcaccagttcgtattatgtgttttacatttacgtgggagtagccgacactttgcttgctatgggattcttcaga
ggtctaccactggtgcatactctaatcacagtgtcgaaaattttacaccacaaaatgttacattctgttcttcaagcacctatgtcaac
cctcaacacgttgaaagcaggtgggattcttaatagattctccaaagatatagcaattttggatgaccttctgcctcttaccatatttg
acttcatccagttgttattaattgtgattggagctatagcagttgtcgcagttttacaaccctacatctttgttgcaacagtgccagtgat
agtggcttttattatgttgagagcatatttcctccaaacctcacagcaactcaaacaactggaatctgaaggcaggagtccaattttc
actcatcttgttacaagcttaaaaggactatggacacttcgtgccttcggacggcagccttactttgaaactctgttccacaaagctc
tgaatttacatactgccaactggttcttgtacctgtcaacactgcgctggttccaaatgagaatagaaatgatttttgtcatcttcttcat
tgctgttaccttcatttccattttaacaacaggagaaggagaaggaagagttggtattatcctgactttagccatgaatatcatgagta
cattgcagtgggctgtaaactccagcatagatgtggatagcttgatgcgatctgtgagccgagtctttaagttcattgacatgccaa
cagaaggtaaacctaccaagtcaaccaaaccatacaagaatggccaactctcgaaagttatgattattgagaattcacacgtgaa
gaaagatgacatctggccctcagggggccaaatgactgtcaaagatctcacagcaaaatacacagaaggtggaaatgccatatt
agagaacatttccttctcaataagtcctggccagagggtgggcctcttgggaagaactggatcagggaagagtactttgttatcag
cttttttgagactactgaacactgaaggagaaatccagatcgatggtgtgtcttgggattcaataactttgcaacagtggaggaaag
cctttggagtgataccacagaaagtatttattttttctggaacatttagaaaaaacttggatccctatgaacagtggagtgatcaaga
aatatggaaagttgcagatgaggttgggctcagatctgtgatagaacagtttcctgggaagcttgactttgtccttgtggatggggg
ctgtgtcctaagccatggccacaagcagttgatgtgcttggctagatctgttctcagtaaggcgaagatcttgctgcttgatgaacc
cagtgctcatttggatccagtaacataccaaataattagaagaactctaaaacaagcatttgctgattgcacagtaattctctgtgaa
cacaggatagaagcaatgctggaatgccaacaatttttggtcatagaagagaacaaagtgcggcagtacgattccatccagaaa
ctgctgaacgagaggagcctcttccggcaagccatcagcccctccgacagggtgaagctctttccccaccggaactcaagcaa
gtgcaagtctaagccccagattgctgctctgaaagaggagacagaagaagaggtgcaagatacaaggctttga
H. s. CFTR amino acid sequence (SEQ ID NO: 2):
MQRSPLEKASVVSKLFFSWTRPILRKGYRQRLELSDIYQIPSVDSADNLSEKLERE
WDRELASKKNPKLINALRRCFFWRFMFYGIFLYLGEVTKAVQPLLLGRIIASYDP
DNKEERSIAIYLGIGLCLLFIVRTLLLHPAIFGLHHIGMQMRIAMFSLIYKKTLKLS
SRVLDKISIGQLVSLLSNNLNKFDEGLALAHFVWIAPLQVALLMGLIWELLQASA
FCGLGFLIVLALFQAGLGRMMMKYRDQRAGKISERLVITSEMIENIQSVKAYCW
EEAMEKMIENLRQTELKLTRKAAYVRYFNSSAFFFSGFFVVFLSVLPYALIKGIIL
RKIFTTISFCIVLRMAVTRQFPWAVQTWYDSLGAINKIQDFLQKQEYKTLEYNLT
TTEVVMENVTAFWEEGFGELFEKAKQNNNNRKTSNGDDSLFFSNFSLLGTPVLK
DINFKIERGQLLAVAGSTGAGKTSLLMVIMGELEPSEGKIKHSGRISFCSQFSWIM
PGTIKENIIFGVSYDEYRYRSVIKACQLEEDISKFAEKDNIVLGEGGITLSGGQRAR
ISLARAVYKDADLYLLDSPFGYLDVLTEKEIFESCVCKLMANKTRILVTSKMEHL
KKADKILILHEGSSYFYGTFSELQNLQPDFSSKLMGCDSFDQFSAERRNSILTETL
HRFSLEGDAPVSWTETKKQSFKQTGEFGEKRKNSILNPINSIRKFSIVQKTPLQMN
GIEEDSDEPLERRLSLVPDSEQGEAILPRISVISTGPTLQARRRQSVLNLMTHSVNQ
GQNIHRKTTASTRKVSLAPQANLTELDIYSRRLSQETGLEISEEINEEDLKECFFDD
MESIPAVTTWNTYLRYITVHKSLIFVLIWCLVIFLAEVAASLVVLWLLGNTPLQD
KGNSTHSRNNSYAVIITSTSSYYVFYIYVGVADTLLAMGFFRGLPLVHTLITVSKI
LHHKMLHSVLQAPMSTLNTLKAGGILNRFSKDIAILDDLLPLTIFDFIQLLLIVIGAI
AVVAVLQPYIFVATVPVIVAFIMLRAYFLQTSQQLKQLESEGRSPIFTHLVTSLKG
LWTLRAFGRQPYFETLFHKALNLHTANWFLYLSTLRWFQMRIEMIFVIFFIAVTFI
SILTTGEGEGRVGIILTLAMNIMSTLQWAVNSSIDVDSLMRSVSRVFKFIDMPTEG
KPTKSTKPYKNGQLSKVMIIENSHVKKDDIWPSGGQMTVKDLTAKYTEGGNAIL
ENISFSISPGQRVGLLGRTGSGKSTLLSAFLRLLNTEGEIQIDGVSWDSITLQQWRK
AFGVIPQKVFIFSGTFRKNLDPYEQWSDQEIWKVADEVGLRSVIEQFPGKLDFVL
VDGGCVLSHGHKQLMCLARSVLSKAKILLLDEPSAHLDPVTYQIIRRTLKQAFAD
CTVILCEHRIEAMLECQQFLVIEENKVRQYDSIQKLLNERSLFRQAISPSDRVKLFP
HRNSSKCKSKPQIAALKEETEEEVQDTRL
Target Sequence 1 (SEQ ID NO: 3):
5′- GTTCTTAAGGCACAGGAACTGGGAC -3′
H. s. CFTR mutant (ΔF508) nucleotide sequence (SEQ ID NO: 4):
atgcagaggtcgcctctggaaaaggccagcgttgtctccaaactttttttcagctggaccagaccaattttgaggaaaggatacag
acagcgcctggaattgtcagacatataccaaatcccttctgttgattctgctgacaatctatctgaaaaattggaaagagaatggga
tagagagctggcttcaaagaaaaatcctaaactcattaatgcccttcggcgatgttttttctggagatttatgttctatggaatctttttat
atttaggggaagtcaccaaagcagtacagcctctcttactgggaagaatcatagcttcctatgacccggataacaaggaggaac
gctctatcgcgatttatctaggcataggcttatgccttctctttattgtgaggacactgctcctacacccagccatttttggccttcatca
cattggaatgcagatgagaatagctatgtttagtttgatttataagaagactttaaagctgtcaagccgtgttctagataaaataagta
ttggacaacttgttagtctcctttccaacaacctgaacaaatttgatgaaggacttgcattggcacatttcgtgtggatcgctcctttgc
aagtggcactcctcatggggctaatctgggagttgttacaggcgtctgccttctgtggacttggtttcctgatagtccttgccctttttc
aggctgggctagggagaatgatgatgaagtacagagatcagagagctgggaagatcagtgaaagacttgtgattacctcagaa
atgattgaaaatatccaatctgttaaggcatactgctgggaagaagcaatggaaaaaatgattgaaaacttaagacaaacagaact
gaaactgactcggaaggcagcctatgtgagatacttcaatagctcagccttcttcttctcagggttctttgtggtgtttttatctgtgctt
ccctatgcactaatcaaaggaatcatcctccggaaaatattcaccaccatctcattctgcattgttctgcgcatggcggtcactcgg
caatttccctgggctgtacaaacatggtatgactctcttggagcaataaacaaaatacaggatttcttacaaaagcaagaatataag
acattggaatataacttaacgactacagaagtagtgatggagaatgtaacagccttctgggaggagggatttggggaattatttga
gaaagcaaaacaaaacaataacaatagaaaaacttctaatggtgatgacagcctcttcttcagtaatttctcacttcttggtactcct
gtcctgaaagatattaatttcaagatagaaagaggacagttgttggcggttgctggatccactggagcaggcaagacttcacttct
aatggtgattatgggagaactggagccttcagagggtaaaattaagcacagtggaagaatttcattctgttctcagttttcctggatt
atgcctggcaccattaaagaaaatatcatcggtgtttcctatgatgaatatagatacagaagcgtcatcaaagcatgccaactaga
agaggacatctccaagtttgcagagaaagacaatatagttcttggagaaggtggaatcacactgagtggaggtcaacgagcaa
gaatttctttagcaagagcagtatacaaagatgctgatttgtatttattagactctccttttggatacctagatgttttaacagaaaaaga
aatatttgaaagctgtgtctgtaaactgatggctaacaaaactaggattttggtcacttctaaaatggaacatttaaagaaagctgac
aaaatattaattttgcatgaaggtagcagctatttttatgggacattttcagaactccaaaatctacagccagactttagctcaaaact
catgggatgtgattctttcgaccaatttagtgcagaaagaagaaattcaatcctaactgagaccttacaccgtttctcattagaagga
gatgctcctgtctcctggacagaaacaaaaaaacaatcttttaaacagactggagagtttggggaaaaaaggaagaattctattct
caatccaatcaactctatacgaaaattttccattgtgcaaaagactcccttacaaatgaatggcatcgaagaggattctgatgagcc
tttagagagaaggctgtccttagtaccagattctgagcagggagaggcgatactgcctcgcatcagcgtgatcagcactggccc
cacgcttcaggcacgaaggaggcagtctgtcctgaacctgatgacacactcagttaaccaaggtcagaacattcaccgaaaga
caacagcatccacacgaaaagtgtcactggcccctcaggcaaacttgactgaactggatatatattcaagaaggttatctcaaga
aactggcttggaaataagtgaagaaattaacgaagaagacttaaaggagtgcttttttgatgatatggagagcataccagcagtga
ctacatggaacacataccttcgatatattactgtccacaagagcttaatttttgtgctaatttggtgcttagtaatttttctggcagaggt
ggctgcttctttggttgtgctgtggctccttggaaacactcctcttcaagacaaagggaatagtactcatagtagaaataacagctat
gcagtgattatcaccagcaccagttcgtattatgtgttttacatttacgtgggagtagccgacactttgcttgctatgggattcttcaga
ggtctaccactggtgcatactctaatcacagtgtcgaaaattttacaccacaaaatgttacattctgttcttcaagcacctatgtcaac
cctcaacacgttgaaagcaggtgggattcttaatagattctccaaagatatagcaattttggatgaccttctgcctcttaccatatttg
acttcatccagttgttattaattgtgattggagctatagcagttgtcgcagttttacaaccctacatctttgttgcaacagtgccagtgat
agtggcttttattatgttgagagcatatttcctccaaacctcacagcaactcaaacaactggaatctgaaggcaggagtccaattttc
actcatcttgttacaagcttaaaaggactatggacacttcgtgccttcggacggcagccttactttgaaactctgttccacaaagctc
tgaatttacatactgccaactggttcttgtacctgtcaacactgcgctggttccaaatgagaatagaaatgatttttgtcatcttcttcat
tgctgttaccttcatttccattttaacaacaggagaaggagaaggaagagttggtattatcctgactttagccatgaatatcatgagta
cattgcagtgggctgtaaactccagcatagatgtggatagcttgatgcgatctgtgagccgagtctttaagttcattgacatgccaa
cagaaggtaaacctaccaagtcaaccaaaccatacaagaatggccaactctcgaaagttatgattattgagaattcacacgtgaa
gaaagatgacatctggccctcagggggccaaatgactgtcaaagatctcacagcaaaatacacagaaggtggaaatgccatatt
agagaacatttccttctcaataagtcctggccagagggtgggcctcttgggaagaactggatcagggaagagtactttgttatcag
cttttttgagactactgaacactgaaggagaaatccagatcgatggtgtgtcttgggattcaataactttgcaacagtggaggaaag
cctttggagtgataccacagaaagtatttattttttctggaacatttagaaaaaacttggatccctatgaacagtggagtgatcaaga
aatatggaaagttgcagatgaggttgggctcagatctgtgatagaacagtttcctgggaagcttgactttgtccttgtggatggggg
ctgtgtcctaagccatggccacaagcagttgatgtgcttggctagatctgttctcagtaaggcgaagatcttgctgcttgatgaacc
cagtgctcatttggatccagtaacataccaaataattagaagaactctaaaacaagcatttgctgattgcacagtaattctctgtgaa
cacaggatagaagcaatgctggaatgccaacaatttttggtcatagaagagaacaaagtgcggcagtacgattccatccagaaa
ctgctgaacgagaggagcctcttccggcaagccatcagcccctccgacagggtgaagctctttccccaccggaactcaagcaa
gtgcaagtctaagccccagattgctgctctgaaagaggagacagaagaagaggtgcaagatacaaggctttga
YFP mutant (meYFP- H148Q/I152L) nucleotide sequence (SEQ ID NO: 5):
atggtgagcaagggcgaggagctgttcaccggggtggtgcccatcctggtcgagctggacggcgacgtaaacggccacaag
ttcagcgtgtccggcgagggcgagggcgatgccacctacggcaagctgaccctgaagttcatctgcaccaccggcaagctgc
ccgtgccctggcccaccctcgtgaccaccttcggctacggcctgcagtgcttcgcccgctaccccgaccacatgaagcagcac
gacttcttcaagtccgccatgcccgaaggctacgtccaggagcgcaccatcttcttcaaggacgacggcaactacaagacccg
cgccgaggtgaagttcgagggcgacaccctggtgaaccgcatcgagctgaagggcatcgacttcaaggaggacggcaacat
cctggggcacaagctggagtacaactacaacagccaaaacgtctatctcatggccgacaagcagaagaacggcatcaaggtg
aacttcaagatccgccacaacatcgaggacggcagcgtgcagctcgccgaccactaccagcagaacacccccatcggcgac
ggccccgtgctgctgcccgacaaccactacctgagctaccagtccgccctgagcaaagaccccaacgagaagcgcgatcaca
tggtcctgctggagttcgtgaccgccgccgggatcactctcggcatggacgagctgtacaagtaa
Signaling Probe 1 (SEQ ID NO: 6):
5′- Cy5 GCCAGTCCCAGTTCCTGTGCCTTAAGAACCTCGC BHQ2 -3′
H. s. CFTR mutant (ΔF508) amino acid sequence (SEQ ID NO: 7):
MQRSPLEKASVVSKLFFSWTRPILRKGYRQRLELSDIYQIPSVDSADNLSEKLERE
WDRELASKKNPKLINALRRCFFWRFMFYGIFLYLGEVTKAVQPLLLGRIIASYDP
DNKEERSIAIYLGIGLCLLFIVRTLLLHPAIFGLHHIGMQMRIAMFSLIYKKTLKLS
SRVLDKISIGQLVSLLSNNLNKFDEGLALAHFVWIAPLQVALLMGLIWELLQASA
FCGLGFLIVLALFQAGLGRMMMKYRDQRAGKISERLVITSEMIENIQSVKAYCW
EEAMEKMIENLRQTELKLTRKAAYVRYFNSSAFFFSGFFVVFLSVLPYALIKGIIL
RKIFTTISFCIVLRMAVTRQFPWAVQTWYDSLGAINKIQDFLQKQEYKTLEYNLT
TTEVVMENVTAFWEEGFGELFEKAKQNNNNRKTSNGDDSLFFSNFSLLGTPVLK
DINFKIERGQLLAVAGSTGAGKTSLLMVIMGELEPSEGKIKHSGRISFCSQFSWIM
PGTIKENIIGVSYDEYRYRSVIKACQLEEDISKFAEKDNIVLGEGGITLSGGQRARI
SLARAVYKDADLYLLDSPFGYLDVLTEKEIFESCVCKLMANKTRILVTSKMEHL
KKADKILILHEGSSYFYGTFSELQNLQPDFSSKLMGCDSFDQFSAERRNSILTETL
HRFSLEGDAPVSWTETKKQSFKQTGEFGEKRKNSILNPINSIRKFSIVQKTPLQMN
GIEEDSDEPLERRLSLVPDSEQGEAILPRISVISTGPTLQARRRQSVLNLMTHSVNQ
GQNIHRKTTASTRKVSLAPQANLTELDIYSRRLSQETGLEISEEINEEDLKECFFDD
MESIPAVTTWNTYLRYITVHKSLIFVLIWCLVIFLAEVAASLVVLWLLGNTPLQD
KGNSTHSRNNSYAVIITSTSSYYVFYIYVGVADTLLAMGFFRGLPLVHTLITVSKI
LHHKMLHSVLQAPMSTLNTLKAGGILNRFSKDIAILDDLLPLTIFDFIQLLLIVIGAI
AVVAVLQPYIFVATVPVIVAFIMLRAYFLQTSQQLKQLESEGRSPIFTHLVTSLKG
LWTLRAFGRQPYFETLFHKALNLHTANWFLYLSTLRWFQMRIEMIFVIFFIAVTFI
SILTTGEGEGRVGIILTLAMNIMSTLQWAVNSSIDVDSLMRSVSRVFKFIDMPTEG
KPTKSTKPYKNGQLSKVMIIENSHVKKDDIWPSGGQMTVKDLTAKYTEGGNAIL
ENISFSISPGQRVGLLGRTGSGKSTLLSAFLRLLNTEGEIQIDGVSWDSITLQQWRK
AFGVIPQKVFIFSGTFRKNLDPYEQWSDQEIWKVADEVGLRSVIEQFPGKLDFVL
VDGGCVLSHGHKQLMCLARSVLSKAKILLLDEPSAHLDPVTYQIIRRTLKQAFAD
CTVILCEHRIEAMLECQQFLVIEENKVRQYDSIQKLLNERSLFRQAISPSDRVKLFP
HRNSSKCKSKPQIAALKEETEEEVQDTRL
Target Sequence 2
(SEQ ID NO: 8)
5′- GAAGTTAACCCTGTCGTTCTGCGAC -3′
Signaling Probe 2
(SEQ ID NO: 9)
5′- CY5.5 GCGAGTCGCAGAACGACAGGGTTAACTTCCTCGC BHQ2 -3′

1. A cell or cell line engineered to stably express cystic fibrosis transmembrane conductance regulator (CFTR).

2-3. (canceled)

4. The cell or cell line of claim 1, which
a) is eukaryotic;
b) is mammalian;
c) does not express endogenous CFTR; or
d) is any combination of (a), (b) and (c).

5-7. (canceled)

8. The cell or cell line of claim 1, which produces a Z′ value of at least 0.4, 0.45, 0.5, 0.55, 0.6, 0.65, 0.7, 0.75, 0.8, or 0.85 in an assay.

9. The cell or cell line of claim 1, which is grown or maintained in the absence of selective pressure.

10. The cell or cell line of claim 1, wherein an auto-fluorescent protein is not expressed or wherein the CFTR does not comprise any polypeptide tag.

11-14. (canceled)

15. The cell or cell line of claim 1, wherein the CFTR is selected from the group consisting of:
a) a CFTR polypeptide comprising the amino acid sequence set forth in SEQ ID NO: 2;
b) a CFTR polypeptide comprising an amino acid sequence that is at least 95% identical to SEQ ID NO: 2;
c) a CFTR polypeptide encoded by a nucleic acid that hybridizes under stringent condition to SEQ ID NO: 1; and
d) a CFTR polypeptide encoded by a nucleic acid that is an allelic variant of SEQ ID NO: 1;
e) a CFTR polypeptide comprising the amino acid sequence set forth in SEQ ID NO: 7; and
f) a CFTR polypeptide encoded by a nucleic acid sequence comprising SEQ ID NO: 4.

16. (canceled)

17. The cell or cell line of claim 1, wherein the CFTR is encoded by a nucleic acid selected from the group consisting of:
a) a nucleic acid comprising the sequence set forth in SEQ ID NO: 1;
b) a nucleic acid that hybridizes to a nucleic acid comprising the nucleotide sequence of SEQ ID NO: 1 under stringent conditions;
c) a nucleic acid that encodes a polypeptide comprising the amino acid sequence of SEQ ID NO: 2;
d) a nucleic acid comprising a nucleotide sequence that is at least 95% identical to SEQ ID NO: 1;
e) a nucleic acid that is an allelic variant of SEQ ID NO: 1;
(f) a nucleic acid comprising the sequence set forth in SEQ ID NO: 4; and
(g) a nucleic acid that encodes a polypeptide comprising the amino acid sequence of SEQ ID NO: 7.

18. (canceled)

19. A collection of the cell or cell line of claim 1, wherein the cells or cell lines in the collection express different forms of CFTR.

20. (canceled)

21. The collection of claim 19, wherein the cells or cell lines are matched to share the same physiological property to allow parallel processing.

22-26. (canceled)

27. A method for producing the cell or cell line of claim 1, comprising the steps of:
a) introducing into host cells a nucleic acid encoding CFTR or introducing into host cells one or more nucleic acid sequences that activate expression of endogenous CFTR;
b) introducing into the host cells a molecular beacon that detects the expression of CFTR or the activated CFTR; and
c) isolating a cell that expresses CFTR or the activated CFTR.

28-29. (canceled)

30. The method of claim 27, wherein the host cells:
a) are eukaryotic cells;
b) are mammalian cells;
c) do not express endogenous CFTR endogenously; or
d) are any combination of (a), (b) and (c).

31. The method of claim 27, wherein the CFTR comprises the amino acid sequence set forth in SEQ ID NO: 2 or SEQ ID NO: 7.

32. The method of claim 27, where in the CFTR is encoded by a nucleic acid selected from the group consisting of:
a) a nucleic acid comprising SEQ ID NO: 1;
b) a nucleic acid that hybridizes to a nucleic acid comprising the nucleotide sequence of SEQ ID NO: 1 under stringent conditions;
c) a nucleic acid that encodes a polypeptide comprising the amino acid sequence of SEQ ID NO: 2;
d) a nucleic acid comprising a nucleotide sequence that is at least 95% identical to SEQ ID NO: 1;
e) a nucleic acid that is an allelic variant of SEQ ID NO: 1; and
f) a nucleic acid comprising SEQ ID NO: 4.

33-35. (canceled)

36. The method of claim 27, wherein the cells or cell lines of the collection are produced in parallel.

37. (canceled)

38. A method for identifying a modulator of a CFTR function comprising the steps of
a) exposing the cell or cell line of claim 1 or the collection of claim 19 to a test compound; and
b) detecting in a cell a change in a CFTR function, wherein a change indicates that the test compound is a CFTR modulator.

39-40. (canceled)

41. The method of claim 38, wherein the CFTR is:
a) a CFTR mutant selected from Table 1 or Table 2;
b) the CFTR encoded by a nucleic acid comprising SEQ ID NO: 4; or
c) the CFTR polypeptide comprising the amino acid sequence set forth in SEQ ID NO: 7.

42-43. (canceled)

44. The method of claim 38, wherein the test compound is in a library of small molecules, chemical moieties, polypeptides, antibodies or antibody fragments.

45-46. (canceled)

47. A cell engineered to stably express CFTR at a consistent level over time, the cell made by a method comprising the steps of:
a) providing a plurality of cells that express mRNA(s) encoding the CFTR;
b) dispersing the cells individually into individual culture vessels, thereby providing a plurality of separate cell cultures;
c) culturing the cells under a set of desired culture conditions using automated cell culture methods characterized in that the conditions are substantially identical for each of the separate cell cultures, during which culturing the number of cells per separate cell culture is normalized, and wherein the separate cultures are passaged on the same schedule;
d) assaying the separate cell cultures to measure expression of the CFTR at least twice; and
e) identifying a separate cell culture that expresses the CFTR at a consistent level in both assays, thereby obtaining said cell.

48. A method for isolating a cell that endogenously expresses CFTR comprising:
a) providing a population of cells;
b) introducing into the cells a molecular beacon that detects expression of CFTR; and
c) isolating cells that express CFTR.

49. (canceled)

50. A method for increasing the expression level of a CFTR on the plasma membrane of a cell, comprising contacting the cell with a compound of the formula: 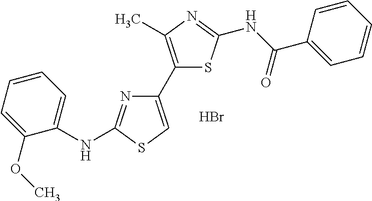
N-{2-[(2-methoxyphenyl)amino]-4′-methyl-4,5′-bi-1,3-thiazol-2′-yl}benzamide hydrobromide.


SEQUENCE LISTING (12 sequences)

SEQ ID NO: 1 - DNA, 4443 bp, Homo sapiens
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttga

SEQ ID NO: 2 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 3 - DNA, 25 bp, Homo sapiens
gttcttaaggcacaggaactgggac

SEQ ID NO: 4 - DNA, 4440 bp, Artificial Sequence
  Description of Artificial Sequence Synthetic polynucleotide
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatcggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttga

SEQ ID NO: 5 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccaaaacgtctatctcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 6 - DNA, 34 bp, Artificial Sequence
  Description of Artificial Sequence Synthetic probe
gccagtcccagttcctgtgccttaagaacctcgc

SEQ ID NO: 7 - protein, 1479 aa, Artificial Sequence
  Description of Artificial Sequence Synthetic polypeptide
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 8 - DNA, 25 bp, Homo sapiens
gaagttaaccctgtcgttctgcgac

SEQ ID NO: 9 - DNA, 34 bp, Artificial Sequence
  Description of Artificial Sequence Synthetic probe
gcgagtcgcagaacgacagggttaacttcctcgc

SEQ ID NO: 10 - DNA, 13 bp, Artificial Sequence
  Description of Artificial Sequence Synthetic oligonucleotide
tgagtactatgag

SEQ ID NO: 11 - DNA, 17 bp, Artificial Sequence
  Description of Artificial Sequence Synthetic oligonucleotide
gtactcaacagctctag

SEQ ID NO: 12 - DNA, 44 bp, Artificial Sequence
  Description of Artificial Sequence Synthetic oligonucleotide
taaataaataaataaataaataaataaataaataaataaataaa